(19) United States
(12) Patent Application Publication
(10) Pub. No.: US 2026/0151349 A1
(43) Pub. Date: Jun. 4, 2026

(54) COMPOSITIONS AND METHODS FOR AUGMENTING BIOHYBRID CHEMICAL SENSOR PERFORMANCE THROUGH NANO-NEUROMODULATION

(71) Applicant: Washington University, St. Louis, MO (US)

(72) Inventors: Srikanth Singamaneni, St. Louis, MO (US); Baranidharan Raman, St. Louis, MO (US); Prashant Gupta, St. Louis, MO (US); Shantanu Chakrabartty, St. Louis, MO (US)

(73) Assignee: Washington University, St. Louis, MO (US)

(21) Appl. No.: 18/977,859

(22) Filed: Dec. 11, 2024

Related U.S. Application Data
(60) Provisional application No. 63/608,679, filed on Dec. 11, 2023.

(51) Int. Cl. A61K 9/50; A61K 9/14; A61K 31/137; A61K 41/00; A61P 25/00

(52) U.S. Cl. CPC A61K 9/5078; A61K 9/143; A61K 9/501; A61K 9/5026; A61K 9/5089; A61K 31/137; A61K 41/0052; A61P 25/00

(57) ABSTRACT

Compositions for photothermally triggered delivery of a neurally active compound and methods of modulating neural activity of preparations containing neurally active cells using the disclosed compositions are disclosed herein. The disclosed compositions include a plurality of functionalized nanoparticles that include a nanoparticle core, a mesoporous coating formed over the nanoparticle core defining a plurality of nanopores, and a mixture of a neurally active compound and a phase change material within the plurality of nanopores. The neurally active compound is selectively released from the composition when the functionalized nanoparticles are heated to a temperature above a melting point of the phase change material. Methods of modulating neural activity include augmenting biohybrid chemical sensors through nano-neuromodulation, and modulation of neural activity of brain organoid preparations.

### CROSS-REFERENCE TO RELATED APPLICATIONS

This application claims priority from U.S. Provisional Application Ser. No. 63/608,679 filed on Dec. 11, 2023, which is incorporated herein by reference in its entirety.

### STATEMENT REGARDING FEDERALLY SPONSORED RESEARCH OR DEVELOPMENT

This invention was made with government support under FA95501910394 awarded by the Air Force Office of Scientific Research (AFOSR) and N000142112343 awarded by the Office of Naval Research (ONR). The government has certain rights in the invention.

### MATERIAL INCORPORATED-BY-REFERENCE

Not applicable.

### FIELD OF THE INVENTION

The present disclosure generally relates to compositions and methods related to augmenting insect olfaction performance through nano-neuromodulation.

### BACKGROUND OF THE INVENTION

Ultrasensitive and rapid chemical detection and quantification are critical for a wide variety of applications, including biodiagnostics, homeland security and environmental monitoring, and industrial process control. However, even after decades of extensive efforts, the performance of artificial chemical sensing systems (‘e-noses’) still pales compared to the superior capabilities of their biological counterparts. On several important metrics such as sensitivity, stability, specificity, and tolerance to varying background conditions, biological olfactory systems exhibit better performance. Hybrid approaches that take advantage of biological capability may be designed, and recent results indicate that such a bio-hybrid or cyborg approach does indeed hold promise for application in security and biomedicine.

Although feasible, developing an approach that taps into the biological capabilities poses several challenges. There are two ways to interrogate a biological system, one involving behavioral observations (such as a dog barking or proboscis extension of insects), or by directly measuring signals from the neural circuits encoding olfactory information. Both these approaches present different sets of challenges that must be overcome to realize a viable bio-hybrid chemical sensing solution. First, behavioral read-outs could be confounding as the generated motor response may also be influenced by other sensory and non-sensory information. On the other hand, neural read-outs provide incomplete information as they are restricted by the number of electrodes that can be placed and the region they can be placed in without compromising the biological system. Furthermore, for the neural read-out approach, it might not be known whether the location at which neural tissue is probed can provide the necessary information for dealing with the chemical sensing problem. For example, in the case of invertebrate olfactory systems, olfactory receptor neurons drive stimulus information-rich spatiotemporal neural activity patterns in the downstream neural circuits (antennal lobe). However, depending upon the location where the neural circuit is probed, the neural readout system might not or only poorly extract these information-rich neural patterns for the stimulus of interest.

Emerging neuromodulation techniques enable fine control over neural network dynamics by regulating the biophysical and synaptic properties of neurons. Among diverse neuromodulation strategies, nanomaterial-assisted non-genetic neuromodulation techniques have gained extensive attention in recent years owing to their superior spatiotemporal resolution, minimal invasiveness, and deep brain stimulation capability. Nanomaterial-assisted neuromodulation wirelessly harnesses energy from deep penetrating external sources (optical, acoustic, and magnetic) and transduces it to physiologically relevant signals recognizable by neurons, thereby allowing for remote biological modulation. Photothermal nanotransducers (such as plasmonic nanostructures, graphene, and polydopamine nanoparticles) that convert optical input into neural perturbations have attracted significant attention recently and in both vertebrate and invertebrate models.

Neurotransmitters play a crucial role in communication between neurons via signal transduction in living organisms. However, considering the rapid deactivation of neurotransmitters by the enzymes in the biological environments, encapsulating the neurotransmitters within a nano-vehicle is a potential solution to ensure long-term stability. Owing to the importance of neurotransmitters in modulating neural activity, the development of nanomaterials that enable on-demand selective and stepwise neurotransmitter release with minimal leakage (i.e., unintended release) is of paramount importance. Among diverse nano-vehicles (lipid-based, polymeric, and inorganic nanoparticles) that enable cargo delivery, mesoporous silica nanoparticles have been widely employed owing to their high pore volume and cargo loading capacity, biocompatibility, and biodegradability. Imparting photothermal properties to mesoporous silica nanoparticles could enable on-demand neurotransmitter release, which in conjunction with controlled non-invasive focal heating can be employed to achieve precise photothermal/chemical modulation of targeted neural networks.

### SUMMARY OF THE INVENTION

Among the various aspects of the present disclosure is the provision of compositions and methods related to modulating neural activity in preparations that include neural cell. Including, but not limited to, biohybrid chemical sensors and brain organoids.

In one aspect, a composition for photothermally triggered delivery of a neurally active compound is disclosed. The composition includes a plurality of functionalized nanoparticles. Each functionalized nanoparticle includes a nanoparticle core with an outer surface, a mesoporous coating formed over the outer surface of the nanoparticle core, and a mixture contained within the plurality of nanopores of the mesoporous coating. The mixture includes the neurally active compound and a phase change material. The mesoporous coating defines the plurality of nanopores. The plurality of nanoparticles is configured to release the neurally active compound from the plurality of pores at a temperature above a melting point of the phase change material. In some aspects, the nanoparticle core includes a biocompatible and biodegradable polymer comprising polydopamine (PDA). In some aspects, the mesoporous coating includes a biocompatible and biodegradable coating material that includes mesoporous silica. In some aspects, the phase change material includes a fatty acid or a fatty alcohol that is immiscible with water and the melting point of the phase change material ranges from about 25° C. to about 60° C. In some aspects, the phase change material is selected from decanoic acid, myristic acid, linolelaidic acid, vaccenic acid, elaidic acid, dodecanol, 1-tridecanol, 1-tetradecanol, hexadecanol, and any combination thereof. In some aspects, the neurally active compound is selected from an ion, a neurotransmitter, a compound to treat a neurologic disorder and any combination thereof. In some aspects, the neurally active compound is selected from a potassium ion (K+), octopamine, glycine, glutamate, serotonin, epinephrine, norepinephrine, dopamine, substance P, an opioid compound, ATP, GTP, nitric oxide, gamma amino butyric acid (GABA), acetylcholine (ACh). In some aspects, the neurally active compound is octopamine. In some aspects, the organic nanoparticle core includes a diameter of about 800 nm. In some aspects, the mesoporous coating includes silica with a thickness of about 120 nm defining a plurality of nanopores that include an average pore diameter of about 3 nm. In some aspects, the nano-vehicle has a zeta potential between −40 and −30 mV.

In another aspect, a method to synthesize a functionalized nanoparticle for thermally activated delivery of a neurally active compound is disclosed. The method includes forming a polydopamine nanoparticle core by oxidative self-polymerization of dopamine monomers in a solution that includes water, ethanol, and ammonium. The method further includes forming a porous silica coating defining a plurality of nanopores over the polydopamine nanoparticle core by incubating the polydopamine nanoparticle in a solution that includes cetyltrimethylammonium bromide, tetraethyl orthosilicate (TEOS), and aqueous ammonia solution. The method further includes forming the functionalized nanoparticle by loading a mixture that includes the neurally active compound and a phase change material into the plurality of nanopores by incubating the nanoparticle in the mixture at a temperature above a melting point of the phase change material. The method further includes storing the nanoparticle in water at a temperature below the melting point of the phase change material to retain the mixture within the plurality of nanopores.

In another aspect, a method of modulating neural activity of a preparation that includes a plurality of neural cells is disclosed. The method includes providing a composition that includes a plurality of functionalized nanoparticles. Each functionalized nanoparticle includes a nanoparticle core encased in a mesoporous coating that includes a plurality of nanopores, and a mixture that includes a neurally active compound and a phase change material contained within the plurality of nanopores of the mesoporous coating. The neurally active compound is configured to modulate the neural activity of the neural cells of the preparation. The method further includes administering a therapeutic amount of the composition to the preparation. The method further includes illuminating the therapeutic amount of the composition with NIR light energy to raise the temperature of the functionalized nanoparticles to a temperature above a melting point of the phase change material to release at least a portion of the neurally active compound to the plurality of neural cells of the preparation. In some aspects, illuminating the therapeutic amount of the composition with NIR light energy further includes using an NIR laser source with a center wavelength of about 800 nm. In some aspects, the NIR laser source produces NIR light energy at a power ranging from about between 8.33 mW/mm² to about 33 mW/mm². In some aspects, the phase change material includes a fatty acid or a fatty alcohol that is immiscible with water and the melting point of the phase change material ranges from about 25° C. to about 60° C. In some aspects, the neurally active compound is selected from an ion, a neurotransmitter, a compound to treat a neurologic disorder and any combination thereof. In some aspects, the preparation is a brain organoid and the neurally active compound is selected from a candidate compound for treatment of a neurological disorder or a neurally active compound associated with a neurological disorder. In some aspects, the preparation is an insect olfaction-based chemical sensor that includes a locust with electrodes monitoring projection neuron activity within an antennal lobe of the locust, and the neurally active compound is octopamine. In some aspects, the preparation is a brain organoid that includes a plurality of neurons, wherein the brain organoid is mounted within an array of electrodes configured to monitor neural activity in different regions of the brain organoid. In some aspects, the method further includes terminating the illumination of the therapeutic amount of the composition with the NIR light energy to lower the temperature of the functionalized nanoparticles to a temperature below the melting point of the phase change material, stopping the release of the neurally active compound from the functionalized nanoparticles; and re-illuminating the therapeutic amount of the composition with NIR light energy to raise the temperature of the functionalized nanoparticles to a temperature above a melting point of the phase change material, to release a second portion of the neurally active compound to the plurality of neural cells of the preparation.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic illustration depicting the loading and photothermally-triggered release of neurotransmitter (octopamine) on nanoparticles for delivery to neural targets.

FIG. 1B is a schematic illustrating the augmentation of locust olfactory response using nanomaterial-assisted neuromodulation of the locust olfactory system to enhance both the odor-evoked spiking response and odor discriminability.

FIG. 2A is a TEM micrograph of PDA nanoparticles.

FIG. 2B is a TEM micrograph of mSiO2@PDA nanoparticles (low magnification).

FIG. 2C is a TEM micrograph of mSiO2@PDA nanoparticles (high magnification).

FIG. 2D is an SEM image of PDA nanoparticles.

FIG. 2E is an SEM image of mSiO2@PDA nanoparticles.

FIG. 2F is a graph of the size distribution of PDA and mSiO2@PDA nanoparticles.

FIG. 2G is a graph of pore size distribution of mesoporous silica layer.

FIG. 2H is an absorbance spectra of PDA and mSiO2@PDA nanoparticles.

FIG. 21 is a graph of Z-potential of PDA and mSiO2@PDA nanoparticles.

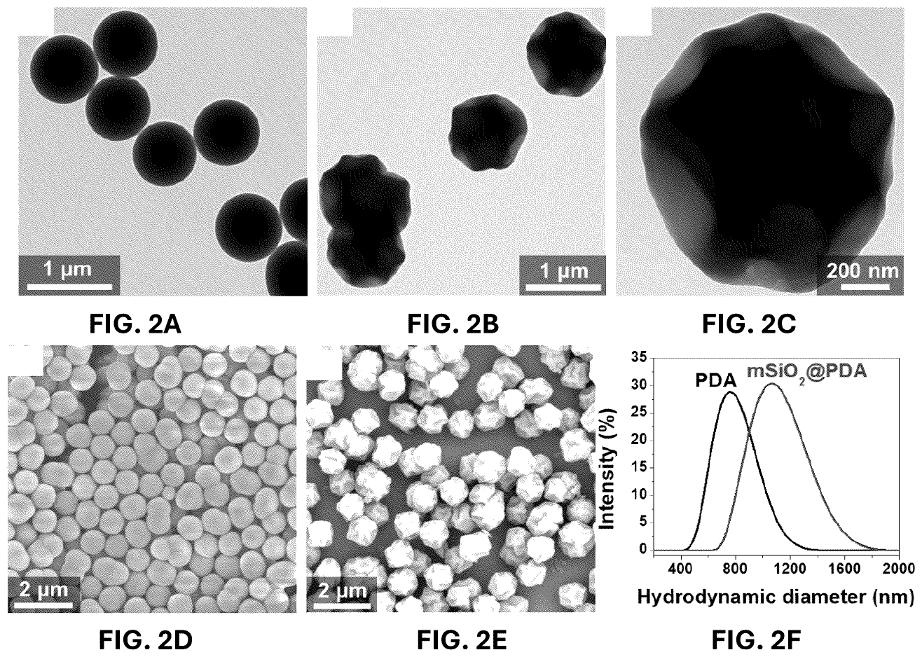

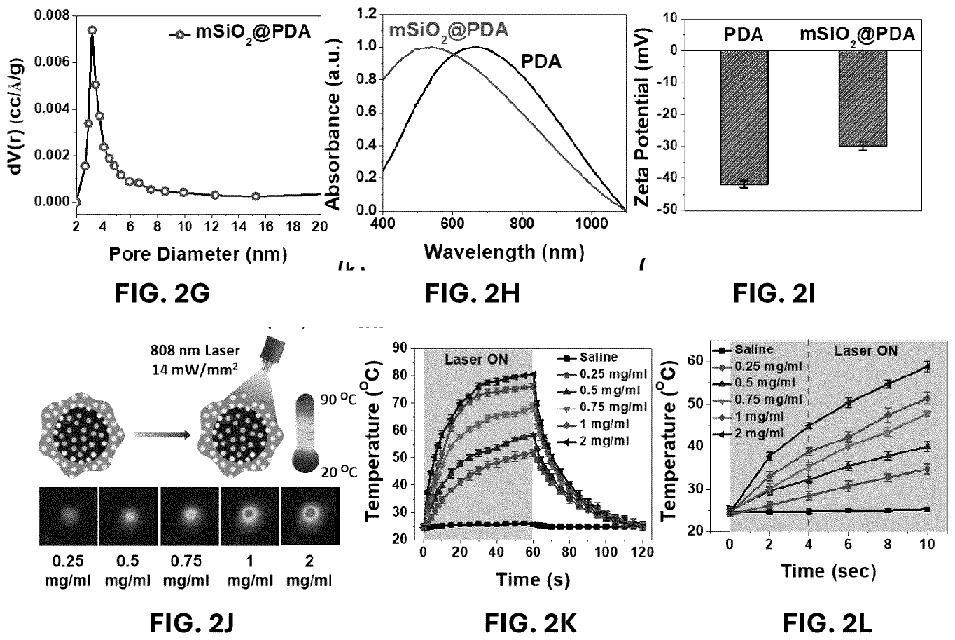

FIG. 2J is a schematic illustrating NIR laser induced photothermal effect of mSiO2@PDA nanoparticles and IR images showing the temperature rise with an increase in particle concentration after 1 minute of NIR illumination.

FIG. 2K is a graph of the temperature rise and fall kinetics of varying concentration of mSiO2@PDA nanoparticles under 1 minute of NIR illumination (power density: 14 mW/mm²).

FIG. 2L is a graph of temperature rise criteria utilized to optimize the particle density in the locust brain for optimal release profile of neurotransmitters (Error bars, s.d., n=3 repeated tests).

FIG. 3A is a schematic illustration of experimental protocol utilized to assess the effect of mSiO2@PDA nanoparticles and the photothermal effect in augmenting locust olfaction.

FIG. 3B is a set of raster plots representing spiking response of three projection neurons (PN) from top to bottom. Each row with a single panel of raster plot corresponds to one trial and responses in ten trials are shown for each PN to illustrate repeatability of the observed effect. The color bar indicates the time when odorants were puffed onto the locust antenna. Responses to hexanol odor stimulus before particle treatment (first column), with NIR laser illumination (power density: 14 mW/mm²) before particle treatment (second column), with 1 mg/ml mSiO2@PDA nanoparticles (third column) and with NIR laser illumination (power density: 14 mW/mm²) in the presence of mSiO2@PDA nanoparticles (fourth column) are shown as individual blocks from left to right.

FIG. 3C is a whisker plot demonstrating increase in on-response (mean spike rate increase in the 4s odor stimulus period) of the locust olfaction under various odor stimulus (hexanol (hex), isoamyl acetate (iaa), 2-octanol (2-oct), benzaldehyde (bza), citral (cit) and cyclohexanone (cyhex)) with NIR laser illumination before particle treatment, with 1 mg/ml mSiO2@PDA nanoparticles and with NIR laser illumination in the presence of mSiO2@PDA nanoparticles compared to pristine condition. Statistical analyses were performed via paired two-samples t-test; n=29 PNs from N=5 locusts, *p<0.05, ** p<0.01, *** p<0.001, and **** p<0.0001. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 3D is a whisker plot demonstrating increase in off-response (mean spike rate increase in the 4s post odor stimulus period) of the locust olfaction under various odor stimulus (hexanol (hex), isoamyl acetate (iaa), 2-octanol (2-oct), benzaldehyde (bza), citral (cit) and cyclohexanone (cyhex)) with NIR laser illumination before particle treatment, with 1 mg/ml mSiO2@PDA nanoparticles and with NIR laser illumination in the presence of mSiO2@PDA nanoparticles compared to pristine condition. Statistical analyses were performed via paired two-samples t-test; n=29 PNs from N=5 locusts, *p<0.05, ** p<0.01, *** p<0.001, and **** p<0.0001. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 3E is a 3D plot of responses of 29 PNs before particle treatment during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 3F is a 3D plot of responses of 29 PNs with NIR laser illumination (power density: 14 mW/mm²) before particle treatment during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 3G is a 3D plot of responses of 29 PNs with 1 mg/ml mSiO2@PDA nanoparticles during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 3H is a 3D plot of responses of 29 PNs with NIR laser illumination (power density: 14 mW/mm²) in the presence of mSiO2@PDA nanoparticles during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 31 is a confusion matrix summarizing the results from 5-fold cross validation classification analyses illustrating odor discrimination capability of locust olfaction under pristine condition. Each row corresponds to the target stimulus and column indicates predicted class.

FIG. 3J is a confusion matrix summarizing the results from 5-fold cross validation classification analyses illustrating odor discrimination capability of locust olfaction with NIR laser illumination before particle treatment. Each row corresponds to the target stimulus and column indicates predicted class.

FIG. 3K is a confusion matrix summarizing the results from 5-fold cross validation classification analyses illustrating odor discrimination capability of locust olfaction with 1 mg/ml mSiO2@PDA nanoparticles. Each row corresponds to the target stimulus and column indicates predicted class.

FIG. 3L is a confusion matrix summarizing the results from 5-fold cross validation classification analyses illustrating odor discrimination capability of locust olfaction with NIR laser illumination in the presence of mSiO2@PDA nanoparticles. Each row corresponds to the target stimulus and column indicates predicted class.

FIG. 4A is a schematic illustrating on-demand photothermally triggered cargo release from neurotransmitter loaded mSiO2@PDA nanoparticles.

FIG. 4B is a graph of release kinetics of NIR laser induced rose bengal (model dye) release from mSiO2@PDA nanoparticles at different time points (individual data points are depicted as black circles and the release kinetics is traced from the mean depicted as red line, n=3 tests).

FIG. 4C is a schematic illustrating dye loaded mSiO2@PDA nanoparticles injection into the locust head through the cuticle and subsequent NIR laser stimulation to assess the dye permeation through the neural sheath to the brain.

FIG. 4D is a set of fluorescence images of the extracted locust brains after different treatments. (1. No particle injection or laser illumination, 2. IR 650 dye loaded mSiO2@PDA nanoparticles injection without laser illumination and 3. IR 650 dye loaded mSiO2@PDA nanoparticles injection with laser illumination). (Individual data points are depicted as black circles, Error bars, s.d., n=3 locusts) Statistical analyses were performed via paired two-samples t-test; *p<0.05, ** p<0.01, *** p<0.001, and **** p<0.0001.

FIG. 4E is a graph of fluorescence intensity of the extracted locust brains after different treatments. (1. No particle injection or laser illumination, 2. IR 650 dye loaded mSiO2@PDA nanoparticles injection without laser illumination and 3. IR 650 dye loaded mSiO2@PDA nanoparticles injection with laser illumination). (Individual data points are depicted as black circles, Error bars, s.d., n=3 locusts) Statistical analyses were performed via paired two-samples t-test; *p<0.05, ** p<0.01, *** p<0.001, and **** p<0.0001.

FIG. 4F is a graph of repeatable NIR-Laser induced on-demand rose bengal (model dye) release from mSiO2@PDA nanoparticles. (Individual data points are depicted as black circles and the release kinetics is traced from the mean depicted as red line, n=3 tests).

FIG. 4G is an 1H NMR spectra of (1) pristine octopamine and (2) supernatant containing released octopamine from octopamine loaded mSiO2@PDA nanoparticles.

FIG. 4H is a graph of release kinetics of NIR laser induced octopamine release from octopamine loaded mSiO2@PDA nanoparticles at different time points (Individual data points are depicted as black circles and the release kinetics is traced from the mean depicted as red 370 line, n=3 tests).

FIG. 5A is a schematic illustration of experimental protocol utilized to assess the effect of octopamine in augmenting locust olfaction.

FIG. 5B is a set of raster plots representing spiking response of three projection neurons (PN) from top to bottom. Each row corresponds to one trial and responses in ten trials are shown for each PN. The color bar indicates the time when odorants were puffed onto the locust antenna. Responses to hexanol odor stimulus pre-octopamine (first column), with 1 mM octopamine (second column) and after washing (third column) are shown as individual blocks from left to right.

FIG. 5C is a whisker plot demonstrating increase in on-response (mean spike rate increase in the 4s odor stimulus period) of the locust olfaction under various odor stimulus (hexanol (hex), isoamyl acetate (iaa), 2-octanol (2-oct), benzaldehyde (bza), citral (cit) and cyclohexanone (cyhex)) in the presence of 1 mM octopamine and after washing with respect to pre-octopamine condition. Statistical analyses were performed via paired two-samples t-test; n=23 PNs from N=5 locusts, *p<0.05, ** p<0.01, *** p<0.001, and **** p<0.0001. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 5D is a whisker plot demonstrating increase in off-response (mean spike rate increase in the 4s post odor stimulus period) of the locust olfaction under various odor stimulus (hexanol (hex), isoamyl acetate (iaa), 2-octanol (2-oct), benzaldehyde (bza), citral (cit) and cyclohexanone (cyhex)) in the presence of 1 mM octopamine and after washing with respect to pre-octopamine condition. Statistical analyses were performed via paired two-samples t-test; n=23 PNs from N=5 locusts, *p<0.05, ** p<0.01, *** p<0.001, and **** p<0.0001. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 5E is a plot of classification performance quantified via 5-fold cross validation analysis is displayed as a confusion matrix illustrating odor discrimination capability of locust olfaction pre-octopamine. Each row corresponds to the target stimulus and column indicates predicted class.

FIG. 5F is a plot of classification performance quantified via 5-fold cross validation analysis is displayed as a confusion matrix illustrating odor discrimination capability of locust olfaction with 1 mM octopamine. Each row corresponds to the target stimulus and column indicates predicted class.

FIG. 5G is a plot of classification performance quantified via 5-fold cross validation analysis is displayed as a confusion matrix illustrating odor discrimination capability of locust olfaction after washing. Each row corresponds to the target stimulus and column indicates predicted class.

FIG. 6A is a schematic illustration of experimental protocol utilized to assess the synergistic effect of photothermally triggered neurotransmitter release in augmenting locust olfaction.

FIG. 6B is a set of raster plots representing spiking response of three projection neurons (PN) from top to bottom are shown. Each row corresponds to one trial and responses in ten trials are shown for each PN. The color bar indicates the time when odorants were puffed onto the locust antenna. Responses to hexanol odor stimulus under pristine condition (first column), with 1 mg/ml octopamine loaded mSiO2@PDA nanoparticles (second column) and with NIR laser (power density: 14 mW/mm²) illumination in the presence of octopamine loaded mSiO2@PDA nanoparticles (third column) are shown as individual blocks from left to right.

FIG. 6C is a set of corresponding mean spike rate of projection neurons representing on and off response to odor stimulus from the raster plots in FIG. 6B. The blue bar indicates the time when odor stimulus was presented to the locust antenna. The gray bar indicates the off-response behavior of projection neurons towards odor stimulus.

FIG. 6D is a whisker plot demonstrating increase in on-response (mean spike rate increase in the 4s odor stimulus period) of the locust olfaction under various odor stimulus (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and with NIR laser illumination in the presence of octopamine loaded mSiO2@PDA nanoparticles compared to pristine condition. Statistical analyses were performed via paired two-samples t-test; n=28 PNs from N=5 locusts, *p<0.05, ** p<0.01, *** p<0.001, and *p<0.0001. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 6E is a whisker plot demonstrating increase in off-response (mean spike rate increase in the 4s post odor stimulus period) of the locust olfaction under various odor stimulus (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and with NIR laser illumination in the presence of octopamine loaded mSiO2@PDA nanoparticles compared to pristine condition. Statistical analyses were performed via paired two-samples t-test; n=28 PNs from N=5 locusts, *p<0.05, ** p<0.01, *** p<0.001, and **** p<0.0001. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 6F is a graph of locust population analyses on the odor recognition performance using (F) on-response, (G) off-response and (H) both on- & off-response, demonstrating robustness of synergistic photothermal/chemical neuromodulation for augmentation of locust olfaction across individual as well as random subsets of locusts. All possible combinations from 5 locusts were considered for the analyses. The mean accuracy was calculated from the accuracy of each iteration (Error bars, s.d, n≥5 iterations, s.d. was calculated from the accuracy of each iteration).

FIG. 6G is a graph of locust population analyses on the odor recognition performance using on-response, demonstrating robustness of synergistic photothermal/chemical neuromodulation for augmentation of locust olfaction across individual as well as random subsets of locusts. All possible combinations from 5 locusts were considered for the analyses. The mean accuracy was calculated from the accuracy of each iteration (Error bars, s.d, n≥5 iterations, s.d. was calculated from the accuracy of each iteration).

FIG. 6H is a graph of locust population analyses on the odor recognition performance using both on- & off-response, demonstrating robustness of synergistic photothermal/chemical neuromodulation for augmentation of locust olfaction across individual as well as random subsets of locusts. All possible combinations from 5 locusts were considered for the analyses. The mean accuracy was calculated from the accuracy of each iteration (Error bars, s.d, n≥5 iterations, s.d. was calculated from the accuracy of each iteration).

FIG. 7 is a set of fluorescent micrographs of human iPSC-derived cerebral organoids.

FIG. 8 is a set of fluorescent micrographs showing positively charged nanoparticles selectively bound to cerebral organoids.

FIG. 9 is a set of fluorescent images showing positively charged nanoparticles bound to cerebral organoids.

FIG. 10 is a set of fluorescent images showing positively charged nanoparticles bound to cerebral organoids.

FIG. 11A is a schematic diagram (left) and image (right) of an experimental preparation that includes a cerebral organoid with bound positively charged nanoparticles as presented in FIGS. 7, 8, and 9 mounted in a support provided with a plurality of electrodes configured to measure reversible photothermal of electrical activity of the organoid.

FIG. 11B is a pair of electrical signal traces produced by the organoid in the apparatus of FIG. 11A, over a time scale of seconds (left) and milliseconds (right).

FIG. 11C is a raster plot (top) and corresponding graph (bottom) of reversible photothermal modulation of electrical activity of the organoid of FIG. 11A induced by 8 mW/mm² of NIR illumination.

FIG. 11D is a raster plot (top) and corresponding graph (bottom) of reversible photothermal modulation of electrical activity of the organoid of FIG. 11A induced by 15 mW/mm² of NIR illumination.

FIG. 11E is a raster plot (top) and corresponding graph (bottom) of reversible photothermal modulation of electrical activity of the organoid of FIG. 11A induced by 33 mW/mm² of NIR illumination.

FIG. 12A is an SEM micrographs of PDA nanoparticles. These are representative images from n=2 independent experiments with similar results.

FIG. 12B is an SEM micrograph of PDA nanoparticles incubated in the similar reaction condition as in mesoporous silica coating but in the absence of TEOS. These are representative images from n=2 independent experiments with similar results.

FIG. 12C is an SEM micrograph of CTAB removed PDA nanoparticles. These are representative images from n=2 independent experiments with similar results.

FIG. 12D is an SEM micrograph (D) SiO2@PDA nanoparticles prior to CTAB removal. These are representative images from n=2 independent experiments with similar results.

FIG. 12E is an SEM micrograph of mSiO2@PDA nanoparticles after CTAB template removal. These are representative images from n=2 independent experiments with similar results.

FIG. 12F is a graph of the quantified corresponding size distribution of nanoparticles via dynamic light scattering of the samples seen in FIG. 12A-E.

FIG. 13 is a raster plot (top panel) representing spiking response of a projection neuron (PN) from is shown. Each row corresponds to one trial and responses in ten trials are shown for one PN. The color bar indicates the time when odorant was puffed onto the locust antenna. Corresponding mean spike rate (bottom panel) of projection neuron representing on and off response to odor stimulus. The blue color bar indicates the time when odor stimulus was presented to the locust antenna. The gray color bar indicates the off-response behavior of projection neurons towards odor stimulus.

FIG. 14A is a set of raster plots representing spiking response of a projection neuron (PN) is shown. Each row corresponds to one trial and responses in ten trials are shown for each odor. The color bar indicates the time when odorants were puffed onto the locust antenna. Responses to various odor stimulus (hexanol (hex), isoamyl acetate (iaa), 2-octanol (2-oct), 12 benzaldehyde (bza), citral (cit) and cyclohexanone (cyhex)) before particle treatment (first column), with NIR laser illumination (power density: 14 mW/mm²) before particle treatment (second column), with 1 mg/ml mSiO2@PDA nanoparticles (third column) and with NIR laser illumination (power density: 14 mW/mm²) in the presence of mSiO2@PDA nanoparticles (fourth column) are shown as individual blocks from left to right.

FIG. 14B is a set of graphs derived from the raster plots shown in FIG. 14A. They show corresponding mean spike rate of projection neuron representing on and off response towards various odor stimulus. The blue color bar indicates the time when odor stimulus was presented to the locust antenna. The gray color bar indicates the off-response behavior of projection neurons towards odor stimulus.

FIG. 15A is a pair of whisker plots demonstrating increase in on-response (mean spike rate increase in the 4s odor stimulus period) and off-response (mean spike rate increase in the 4s post odor stimulus period) of locust 1 under various odor stimulus (hexanol (hex), isoamyl acetate (iaa), 2-octanol (2-oct), benzaldehyde (bza), citral (cit) and cyclohexanone (cyhex)) with NIR laser illumination before particle treatment, with 1 mg/ml mSiO2@PDA nanoparticles and 14 with NIR laser illumination in the presence of mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 15B is a pair of whisker plots demonstrating increase in on-response (mean spike rate increase in the 4s odor stimulus period) and off-response (mean spike rate increase in the 4s post odor stimulus period) of locust 2 under various odor stimulus (hexanol (hex), isoamyl acetate (iaa), 2-octanol (2-oct), benzaldehyde (bza), citral (cit) and cyclohexanone (cyhex)) with NIR laser illumination before particle treatment, with 1 mg/ml mSiO2@PDA nanoparticles and 14 with NIR laser illumination in the presence of mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 15C is a pair of whisker plots demonstrating increase in on-response (mean spike rate increase in the 4s odor stimulus period) and off-response (mean spike rate increase in the 4s post odor stimulus period) of locust 3 under various odor stimulus (hexanol (hex), isoamyl acetate (iaa), 2-octanol (2-oct), benzaldehyde (bza), citral (cit) and cyclohexanone (cyhex)) with NIR laser illumination before particle treatment, with 1 mg/ml mSiO2@PDA nanoparticles and 14 with NIR laser illumination in the presence of mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 15D is a pair of whisker plots demonstrating increase in on-response (mean spike rate increase in the 4s odor stimulus period) and off-response (mean spike rate increase in the 4s post odor stimulus period) of locust 4 under various odor stimulus (hexanol (hex), isoamyl acetate (iaa), 2-octanol (2-oct), benzaldehyde (bza), citral (cit) and cyclohexanone (cyhex)) with NIR laser illumination before particle treatment, with 1 mg/ml mSiO2@PDA nanoparticles and 14 with NIR laser illumination in the presence of mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 15E is a pair of whisker plots demonstrating increase in on-response (mean spike rate increase in the 4s odor stimulus period) and off-response (mean spike rate increase in the 4s post odor stimulus period) of locust 5 under various odor stimulus (hexanol (hex), isoamyl acetate (iaa), 2-octanol (2-oct), benzaldehyde (bza), citral (cit) and cyclohexanone (cyhex)) with NIR laser illumination before particle treatment, with 1 mg/ml mSiO2@PDA nanoparticles and 14 with NIR laser illumination in the presence of mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 16A is a set of graphs of locust population analyses on the increase in on-response demonstrating robustness of the photothermal neuromodulation for augmentation of locust olfaction across individual as well as random subsets of locusts. All possible combinations from 5 locusts were considered for the analyses. The increase in on- and off-response is presented as the mean calculated from the means of each iteration (Data are presented as mean values+/−SD, n=5 iterations (number of locusts=1 or 4) and n=10 iterations (number of locusts=2 or 3), SD was calculated from the accuracy of each iteration).

FIG. 16B is a set of graphs of locust population analyses on the increase in off-response demonstrating robustness of the photothermal neuromodulation for augmentation of locust olfaction across individual as well as random subsets of locusts. All possible combinations from 5 locusts were considered for the analyses. The increase in on- and off-response is presented as the mean calculated from the means of each iteration (Data are presented as mean values+/−SD, n=5 iterations (number of locusts=1 or 4) and n=10 iterations (number of locusts=2 or 3), SD was calculated from the accuracy of each iteration).

FIG. 17A is a 3D plot of the odor evoked responses from all 29 neurons that were combined to generate an ensemble vector (see methods in Example 1), and the activity was binned in 50 ms time bins. To visualize the responses towards various odors and compare them, these high dimensional responses were dimensionality reduced using principal component analysis (PCA) and projected into top three principal component space. High dimensional responses for various odors (hexanol (hex), isoamyl acetate (iaa), 2-octanol (2-oct), benzaldehyde (bza), citral (cit) and cyclohexanone (cyhex)) are shown before particle treatment and how these responses evolve over time are visualized in three dimensions following principal 18 component analysis (see Methods in Example 1). Numbers within parentheses indicate the variance captured along that axis.

FIG. 17B is a 3D plot as described in FIG. 17A with NIR laser illumination (power density: 14 mW/mm²) before particle treatment and how these responses evolve over time are visualized in three dimensions following principal 18 component analysis (see Methods in Example 1). Numbers within parentheses indicate the variance captured along that axis.

FIG. 17C is another 3D plot as described in FIG. 17A with 1 mg/ml mSiO2@PDA nanoparticles and how these responses evolve over time are visualized in three dimensions following principal 18 component analysis (see Methods in Example 1). Numbers within parentheses indicate the variance captured along that axis.

FIG. 17D is yet another 3D as described in FIG. 17A with NIR laser illumination (power density: 14 mW/mm²) in the presence of mSiO2@PDA nanoparticles and how these responses evolve over time are visualized in three dimensions following principal 18 component analysis (see Methods in Example 1). Numbers within parentheses indicate the variance captured along that axis.

FIG. 17E is a 3D plot of the responses of 29 PNs (E) before particle treatment during odor presentation window (50 ms bin size, over 4 s, total 80 points for each odor) that are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 17F is a 3D plot of the responses of 29 PNs (F) with NIR laser illumination (power density: 14 mW/mm²) before particle treatment during odor presentation window (50 ms bin size, over 4 s, total 80 points for each odor) that are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 17G is a 3D plot of the responses of 29 PNs (G) with 1 mg/ml mSiO2@PDA nanoparticles during odor presentation window (50 ms bin size, over 4 s, total 80 points for each odor) that are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 17H is a 3D plot of the responses of 29 PNs (H) with NIR laser illumination (power density: 14 mW/mm²) in the presence of mSiO2@PDA nanoparticles during odor presentation window (50 ms bin size, over 4 s, total 80 points for each odor) that are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 18A is a plot of the correlations between ensemble response vectors evoked by an odorant in different time bins following stimulus onset. Spike counts were averaged across trials (n=10 trials) for all 29 PNs and used for this analysis. Note that each pixel represents correlation between one ensemble vector with another. Similarly, one row or column represents the correlation between one ensemble vector with all other vectors in the identified time periods 20 (80 ON response vectors). Each square with thicker black boundary represents individual odor and each smaller square with thinner black boundary represents response of PNs (1) before particle treatment, (2) with NIR laser illumination (power density: 14 mW/mm²) before particle treatment, (3) with 1 mg/ml mSiO2@PDA nanoparticles and, (4) with NIR laser illumination (power density: 14 mW/mm²) in the presence of mSiO2@PDA nanoparticles. The color scheme used for representing the correlation values is shown on the right.

FIG. 18B is a plot of the correlations between ensemble odor evoked response of PNs, as similarly described in FIG. 18A.

FIG. 19 is a set of graphs showing locust population analyses on odor recognition performance demonstrating robustness of the photothermal neuromodulation for augmentation of locust olfaction across individual as well as random subsets of locusts. All possible combinations from 5 locusts were considered for the analyses. The mean accuracy was calculated from the accuracy of each iteration (Data are presented as mean values+/−SD, n=5 iterations (number of locusts=1 or 4) and n=10 iterations (number of locusts=2 or 3), SD was calculated from the accuracy of each iteration).

FIG. 20A is a schematic illustration of experimental protocol utilized to assess the robustness of locust olfaction over time.

FIG. 20B is a set of raster plots representing spiking response of three projection neurons (PN) from top to bottom are shown. Each row with a single panel of raster plot corresponds to one trial and responses in ten trials are shown for each PN to illustrate repeatability of the observed effect. The color bar 23 indicates the time when odorants were puffed onto the locust antenna. Responses to hexanol odor stimulus at T=0 min (first column), T=90 min (second column), T=210 min (third column) and T=300 min (fourth column) are shown as individual blocks from left to right. The time points were chosen so as to mimic the timeline used for longest recording experiments.

FIG. 20C is a whisker plot demonstrating increase in on-response (mean spike rate increase in the 4s odor stimulus period) of the locust olfaction under various odor stimulus (hexanol (hex), isoamyl acetate (iaa), 2-octanol (2-oct), benzaldehyde (bza), citral (cit) and cyclohexanone (cyhex)) at various time points. Statistical analyses were performed via two-sided paired two-samples t-test; n=23 PNs from N=5 locusts, *p<0.05, ** p<0.01, *** p<0.001, **** p<0.0001. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 20D is a whisker plot demonstrating increase in off-response (mean spike rate increase in the 4s post odor stimulus period) of the locust olfaction as described in FIG. 20D.

FIG. 20E is a 3D plot of responses of 23 PNs at T=0 min during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 20F is a 3D plot of responses of 23 PNs at T=90 min during odor presentation window (50 ms bin size, over 4 s, total 80 points for each odor) are visualized after linear discriminant analysis (LDA) dimensionality reduction.

FIG. 20G is a 3D plot of responses of 23 PNs at T=210 min during odor presentation window (50 ms bin size, over 4 s, total 80 points for each odor) are visualized after linear discriminant analysis (LDA) dimensionality reduction.

FIG. 20H is a 3D plot of responses of 23 PNs at T=300 min during odor presentation window (50 ms bin size, over 4 s, total 80 points for each odor) are visualized after linear discriminant analysis (LDA) dimensionality reduction.

FIG. 20I is a confusion matrix summarizing the results from classification analyses illustrating odor discrimination capability of locust olfaction at T=0 min. Each row corresponds to the target stimulus and column indicates predicted class. Schematics were created with BioRender.com.

FIG. 20J is a confusion matrix summarizing the results from classification analyses illustrating odor discrimination capability of locust olfaction at T=90 min. Each row corresponds to the target stimulus and column indicates predicted class. Schematics were created with BioRender.com.

FIG. 20K is a confusion matrix summarizing the results from classification analyses illustrating odor discrimination capability of locust olfaction at T=210 min. Each row corresponds to the target stimulus and column indicates predicted class. Schematics were created with BioRender.com.

FIG. 20L is a confusion matrix summarizing the results from classification analyses illustrating odor discrimination capability of locust olfaction at T=300 min. Each row corresponds to the target stimulus and column indicates predicted class. Schematics were created with BioRender.com.

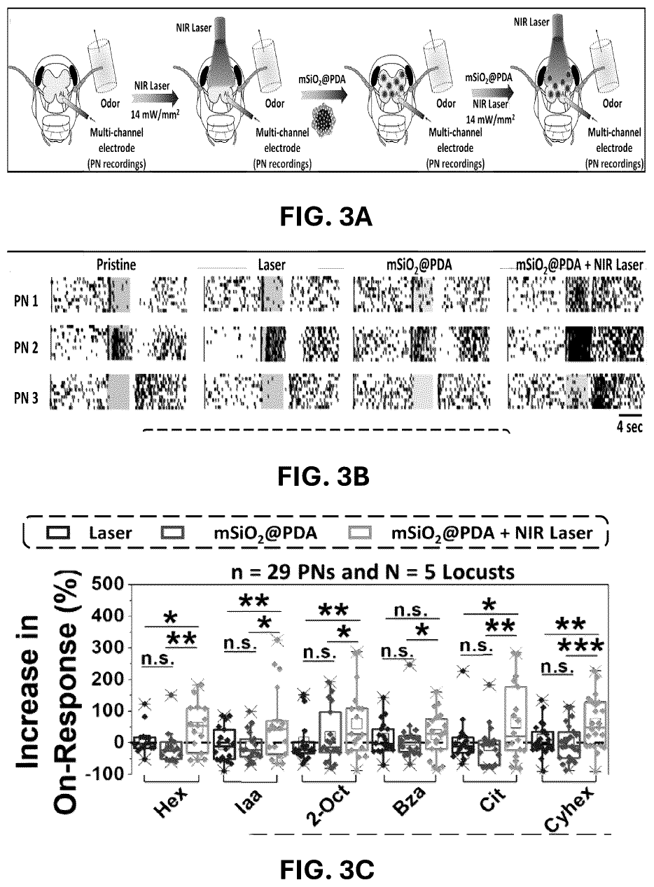

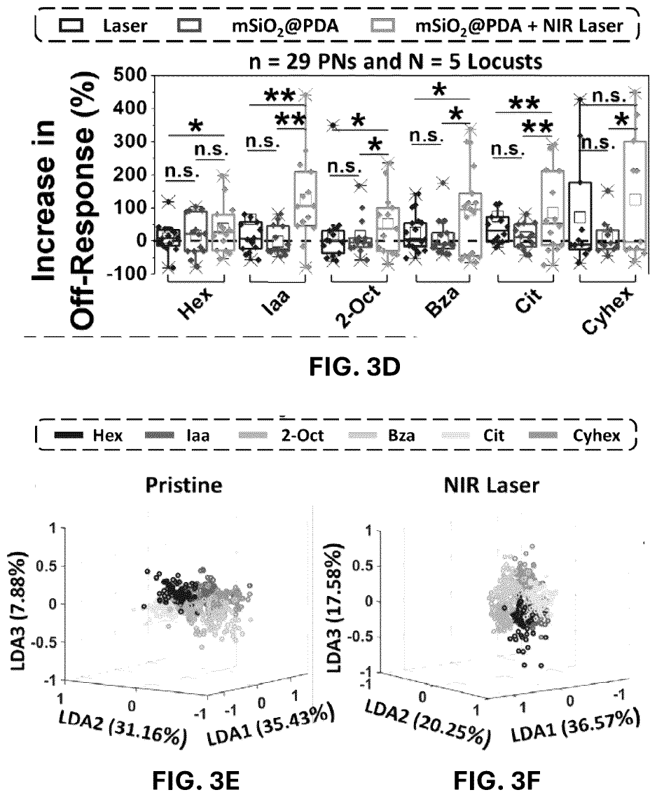

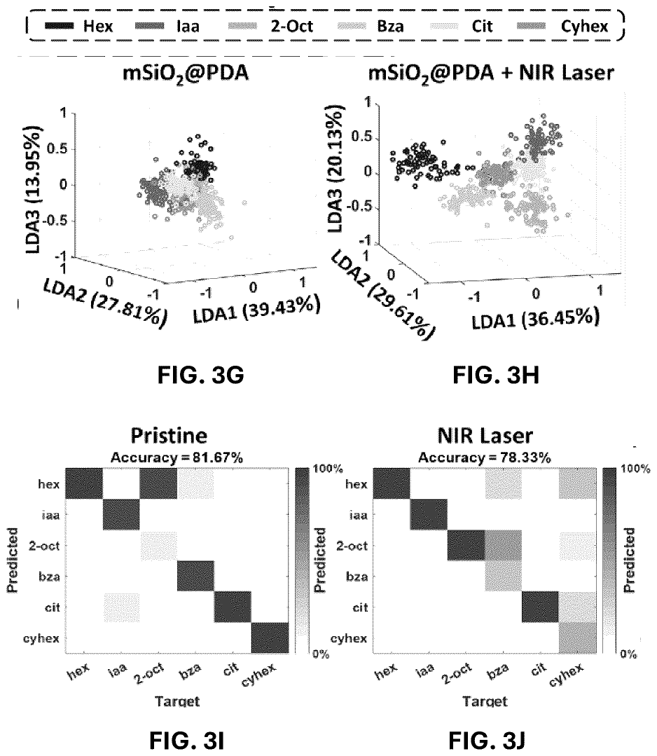

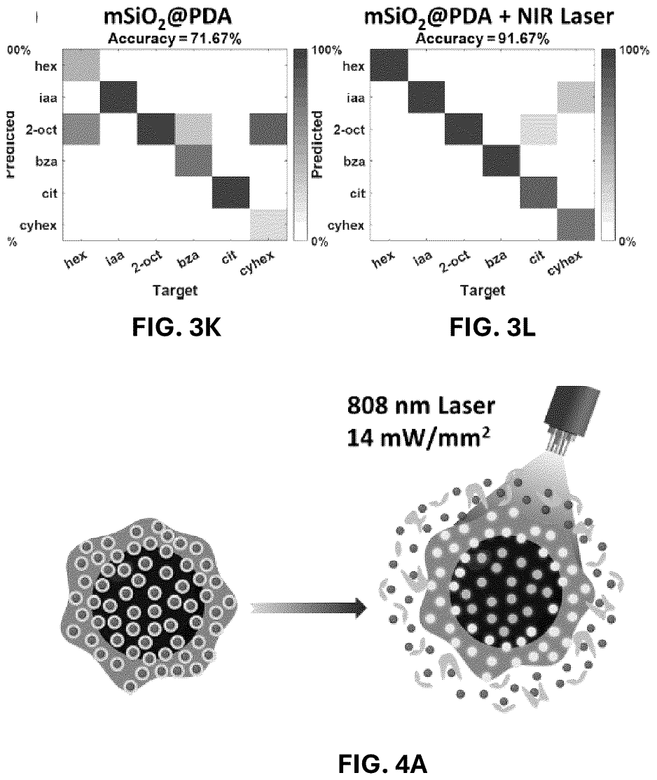

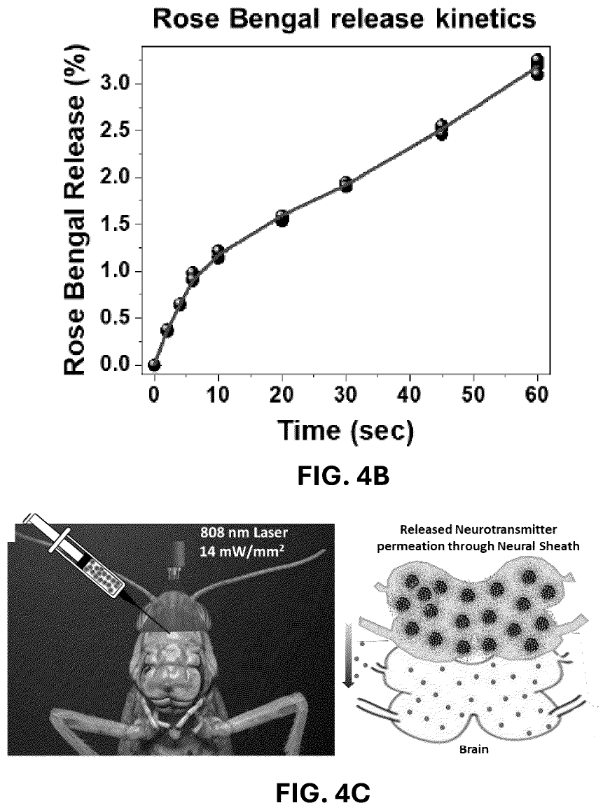

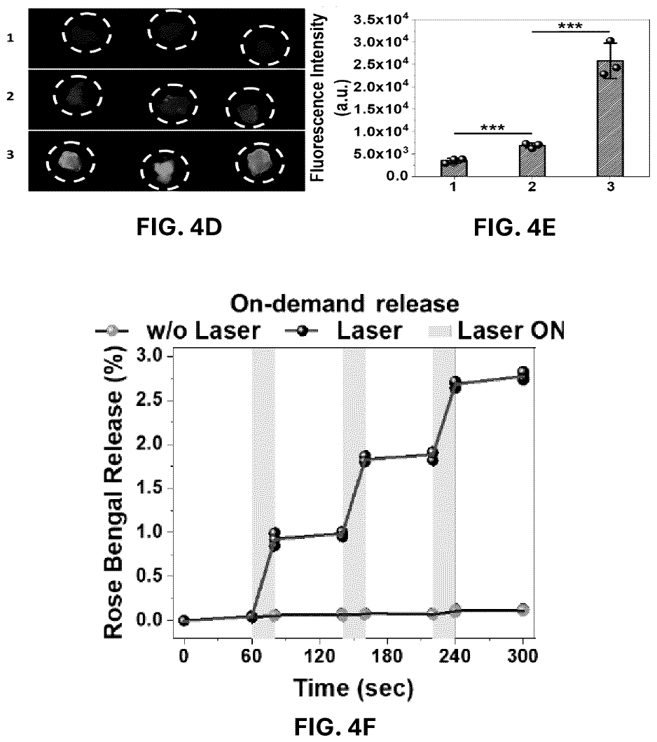

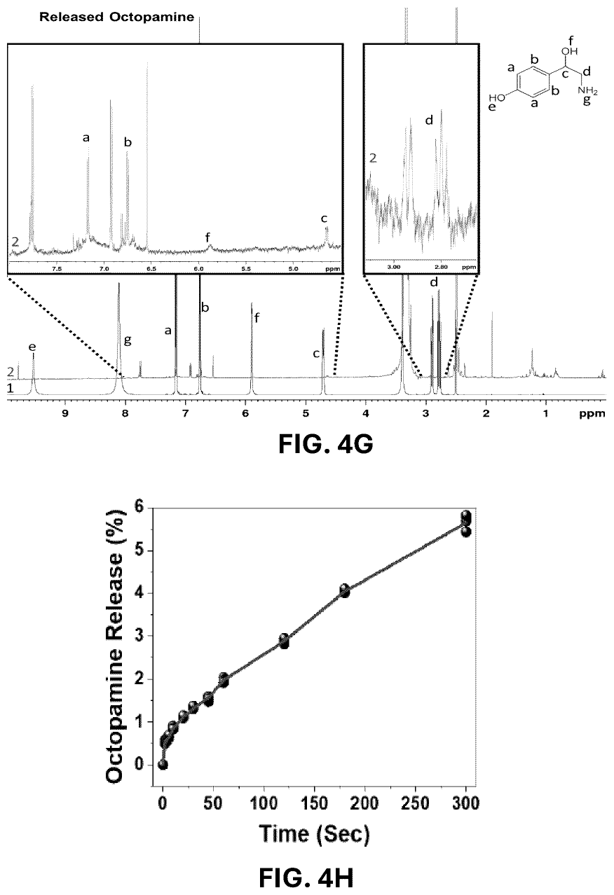

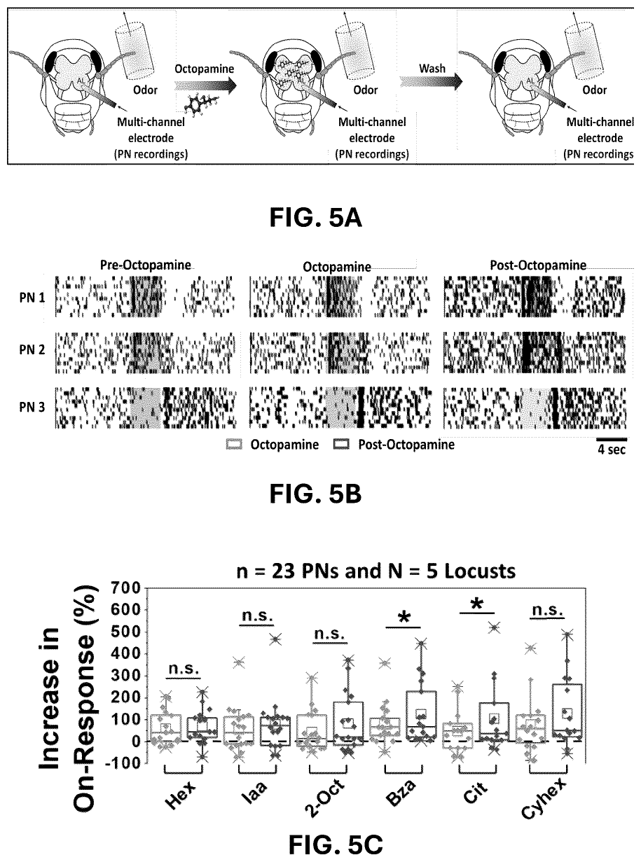

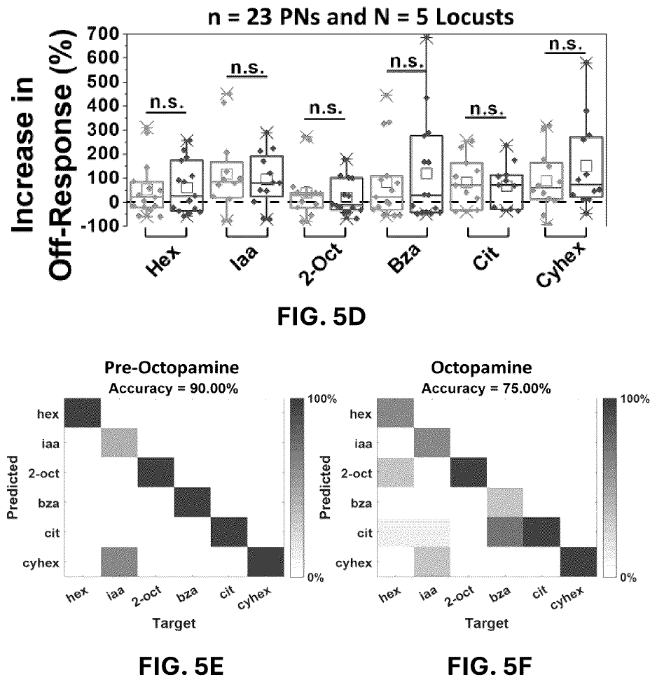

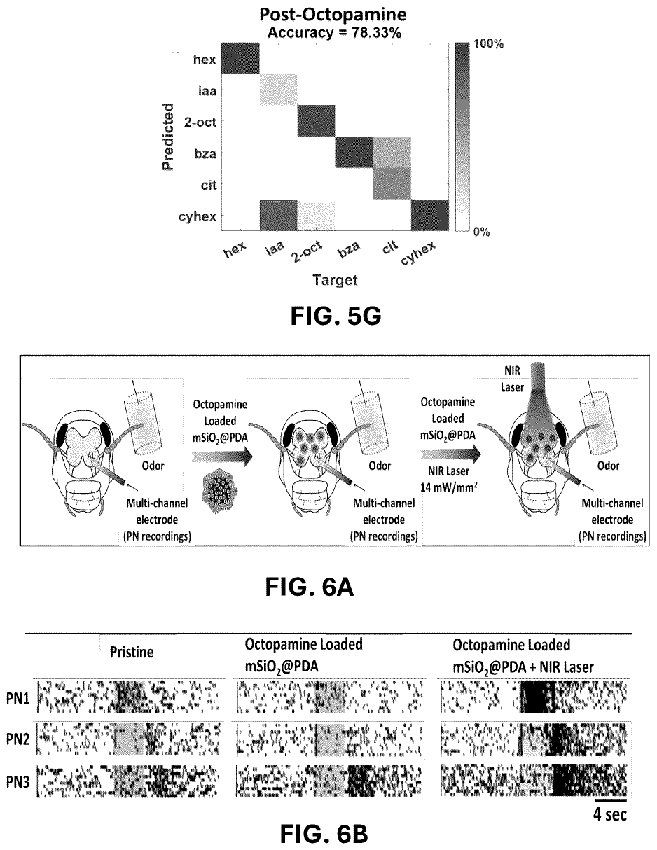

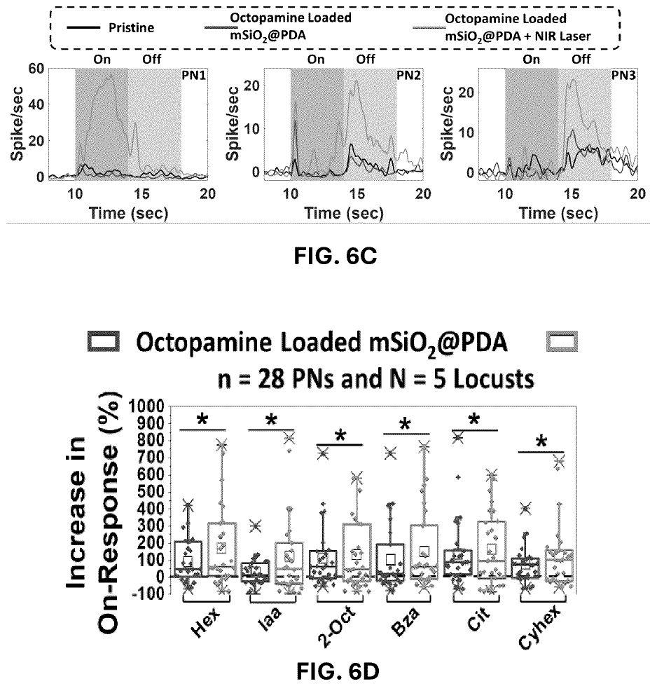

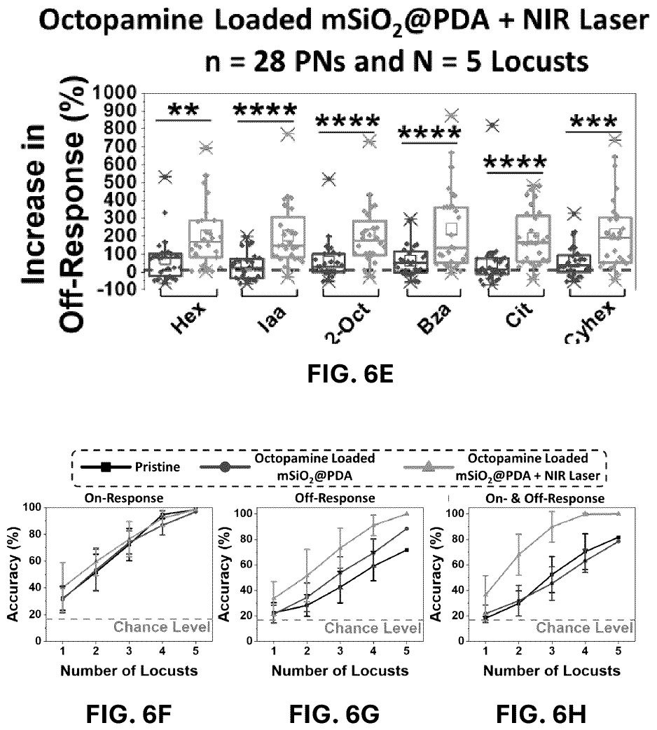

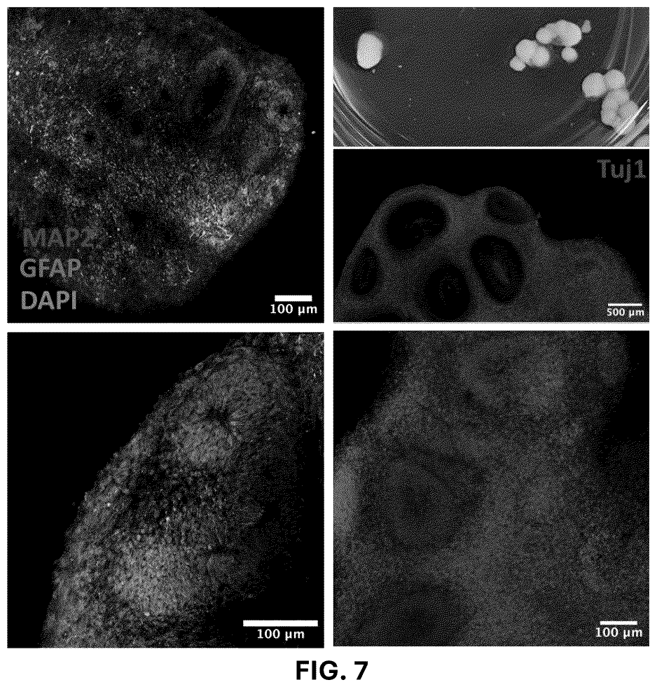

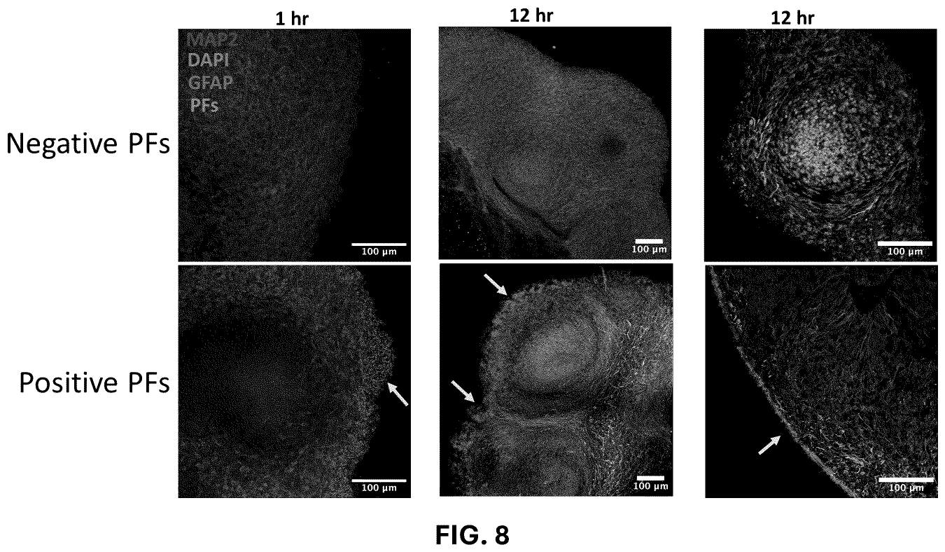

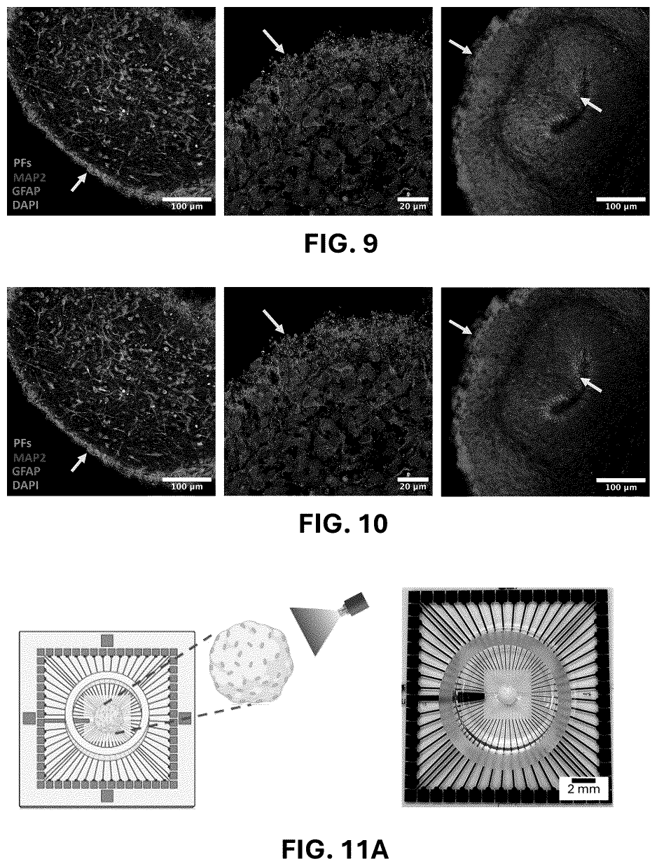

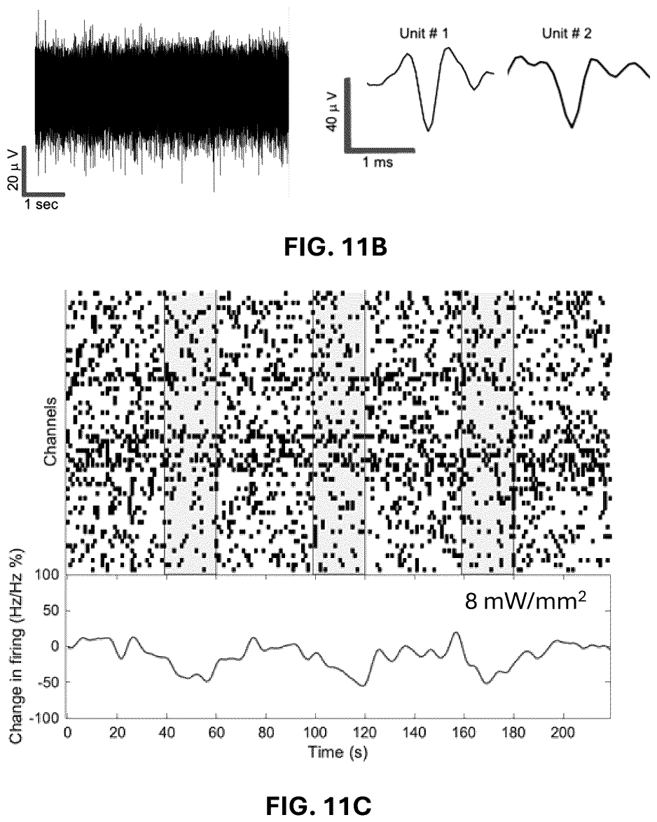

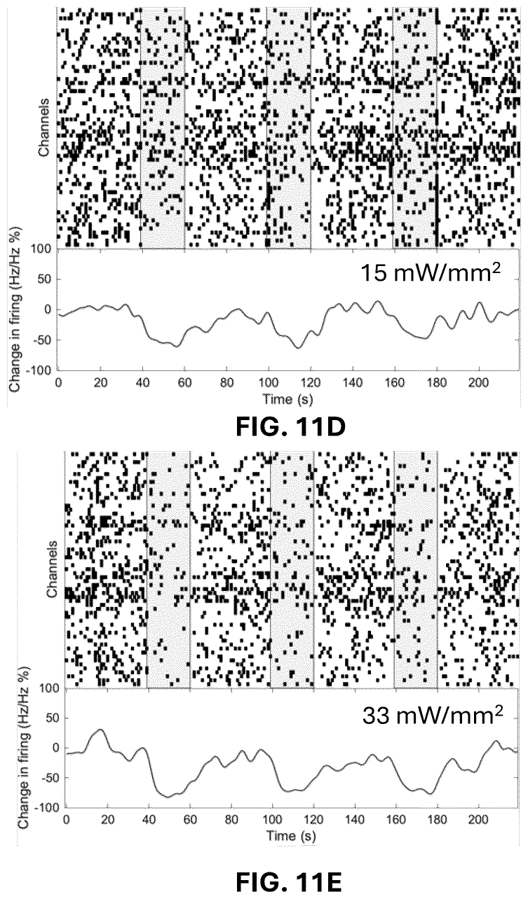

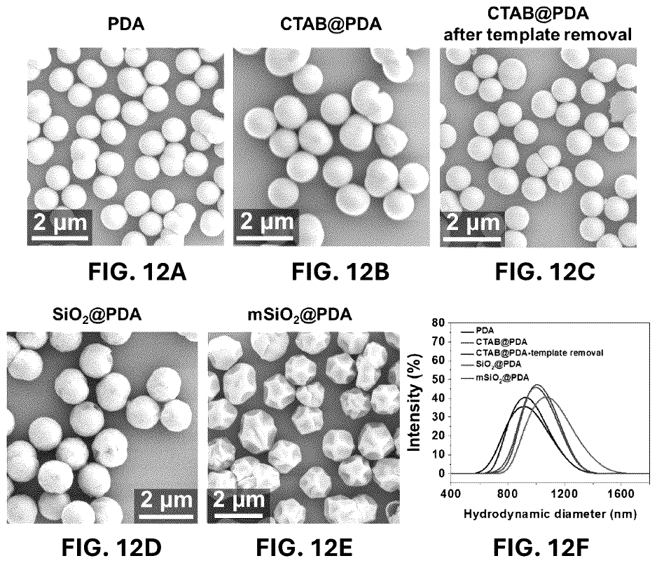

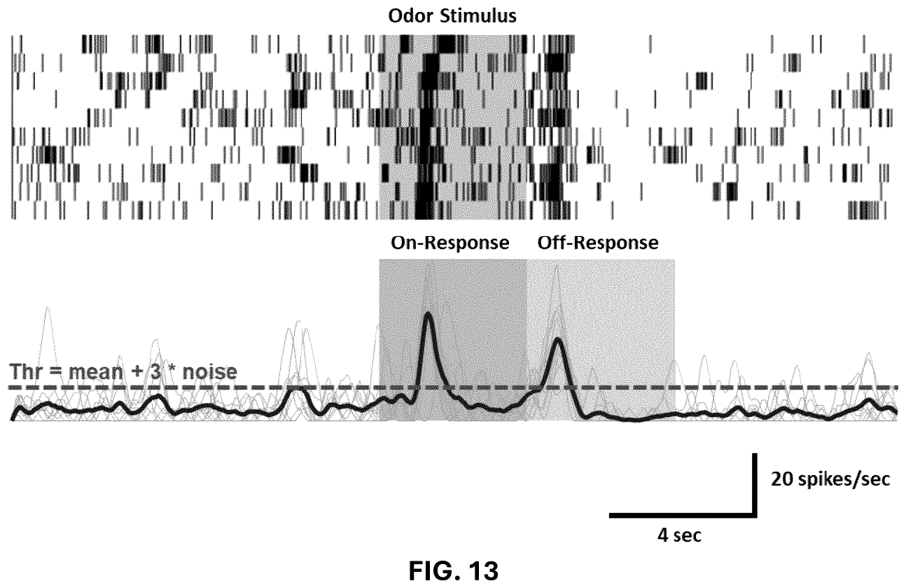

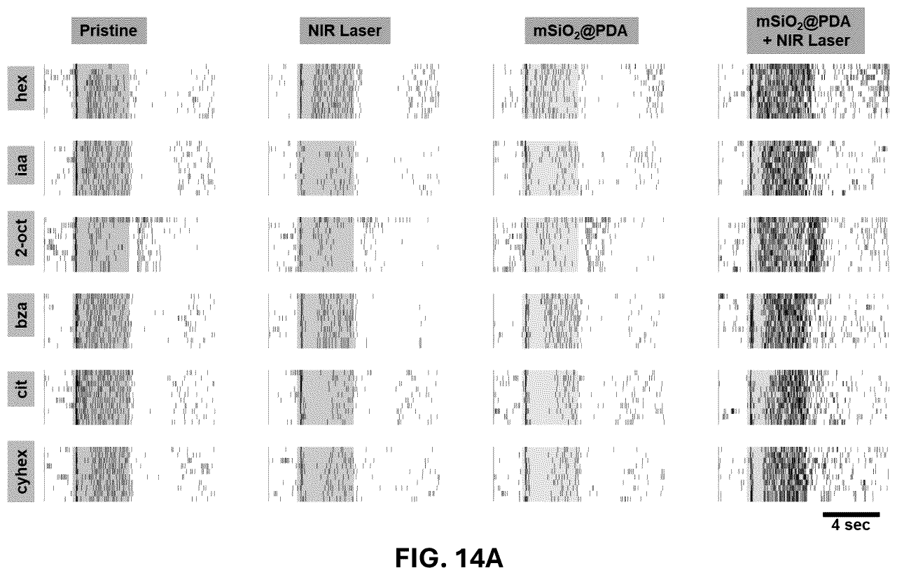

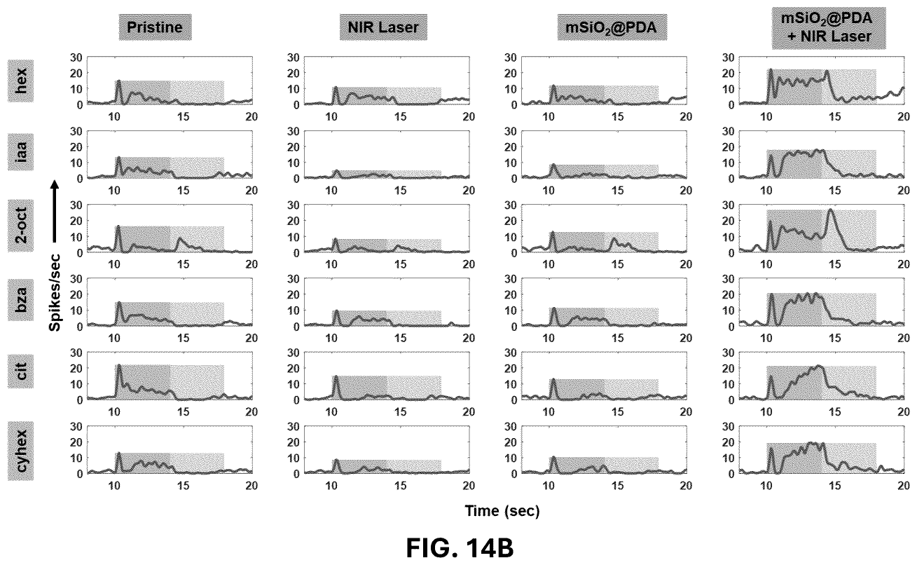

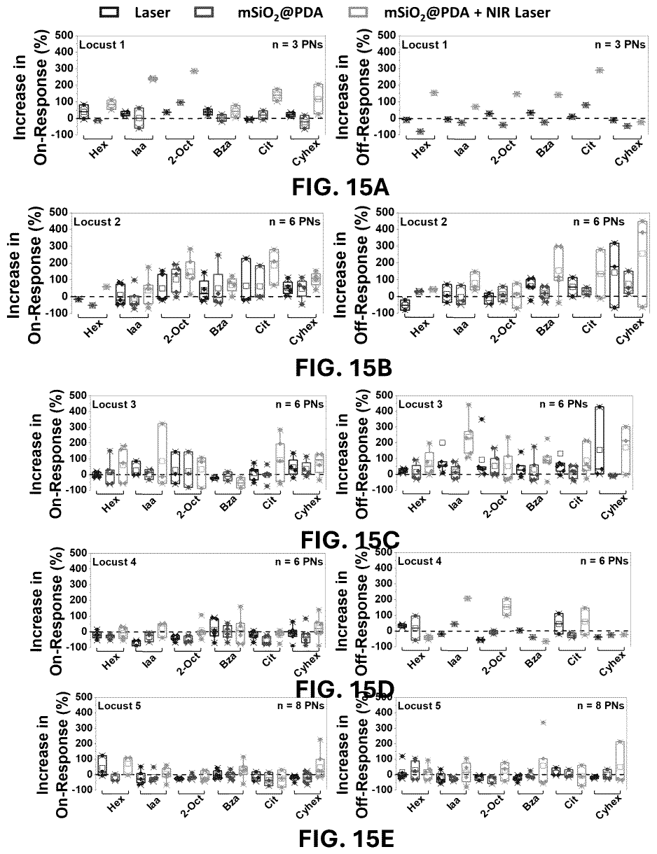

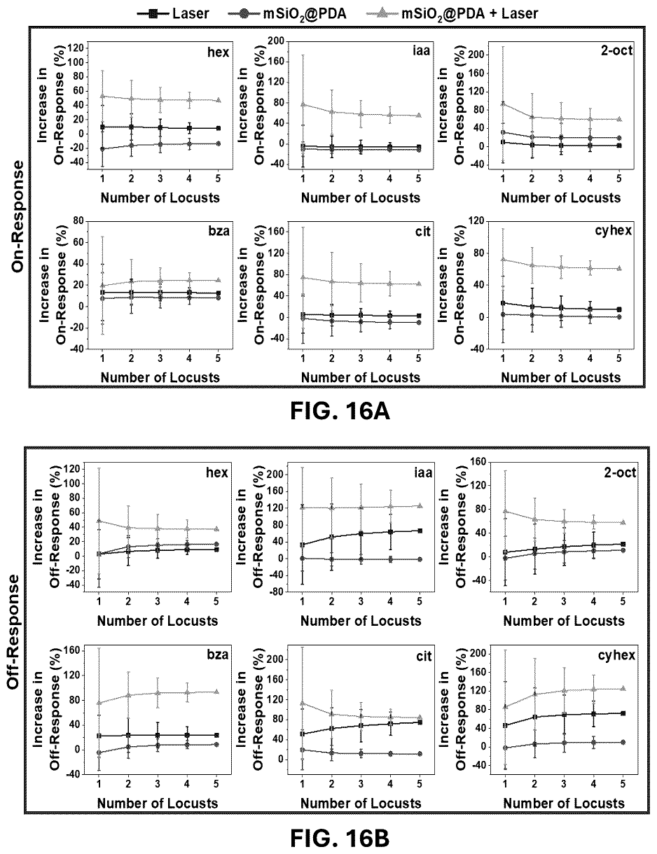

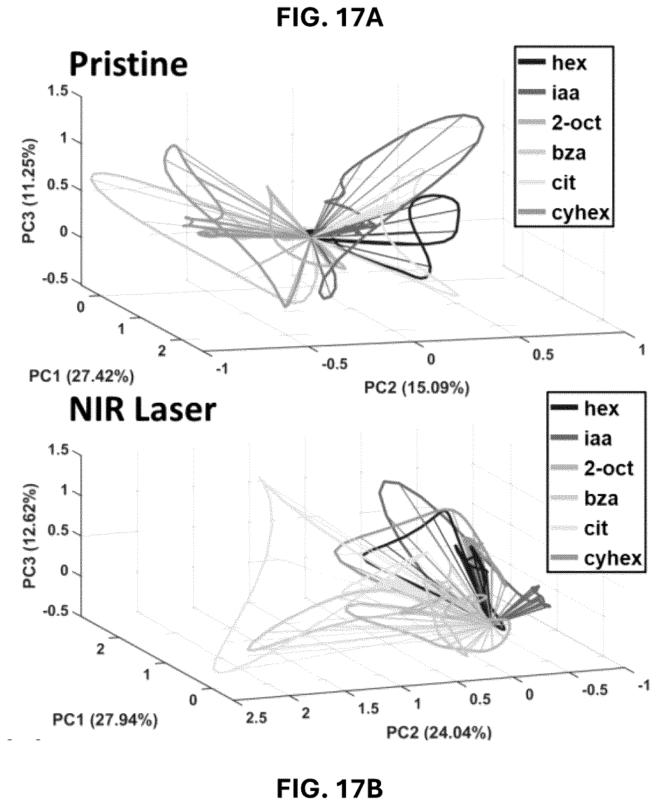

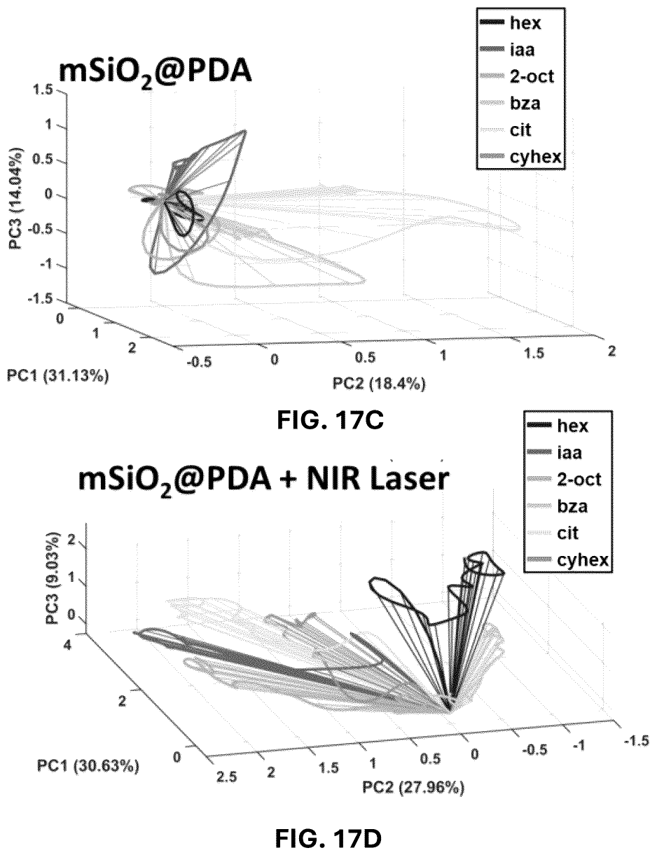

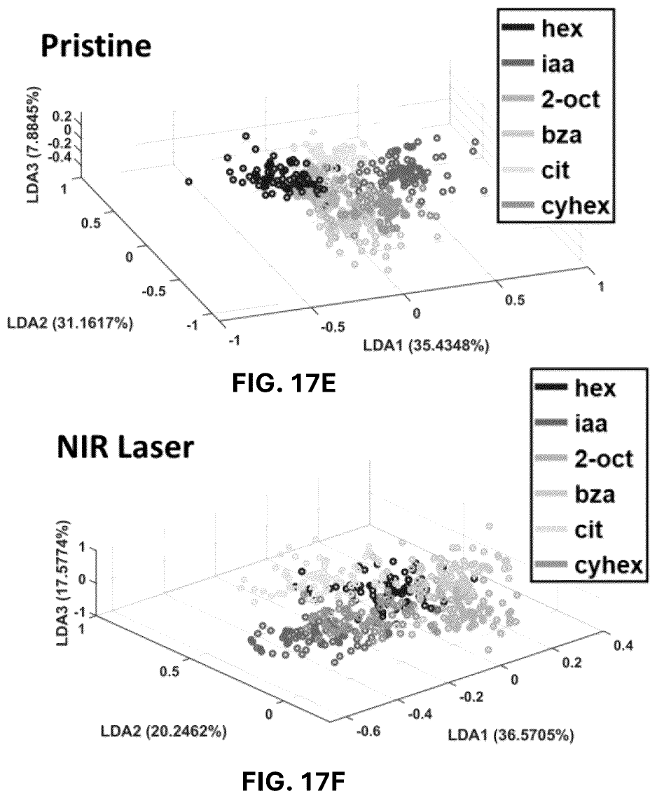

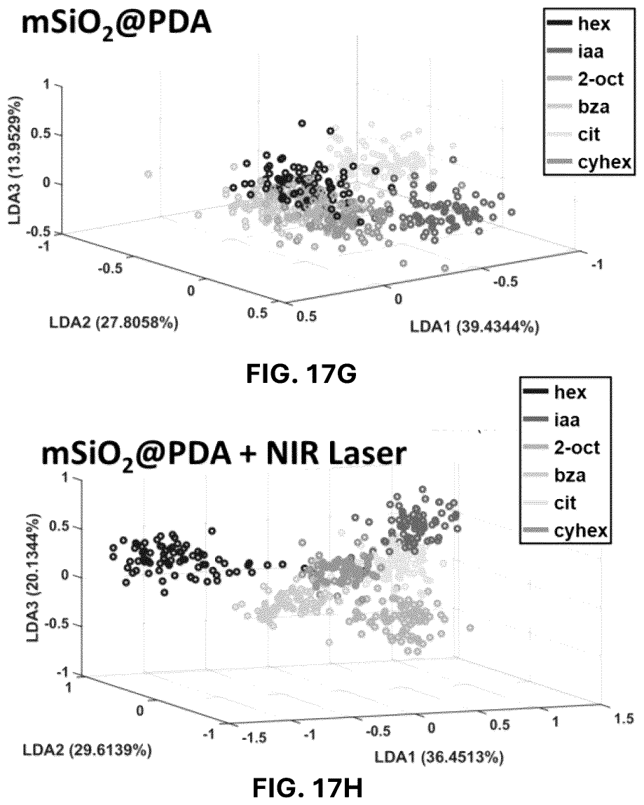

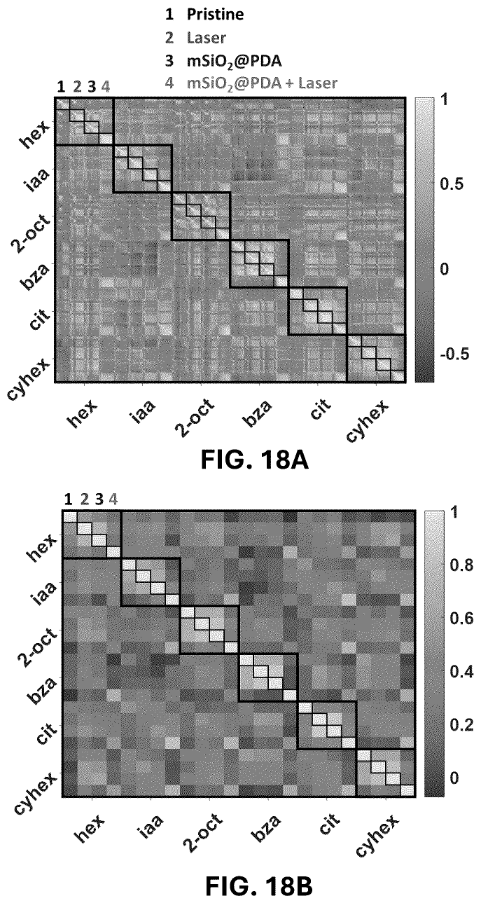

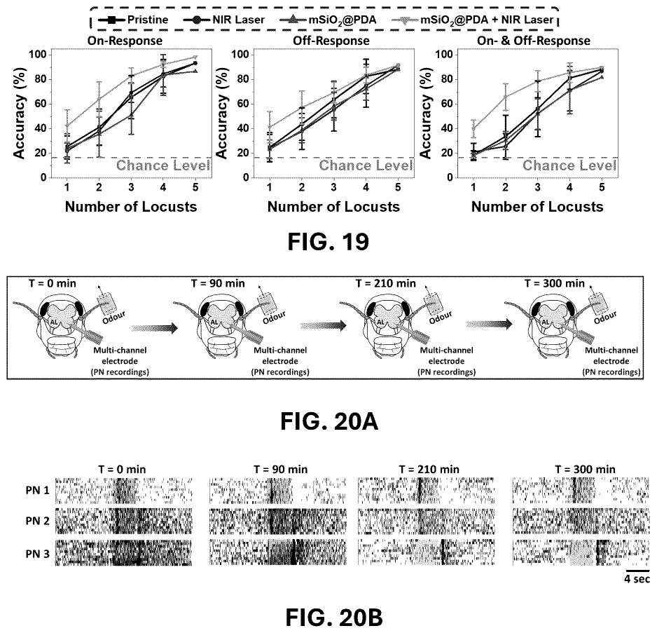

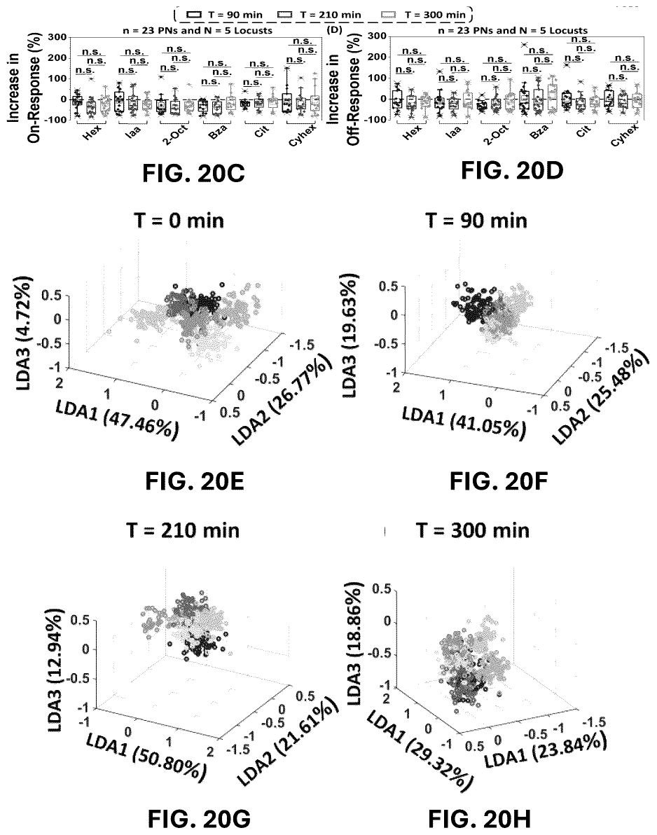

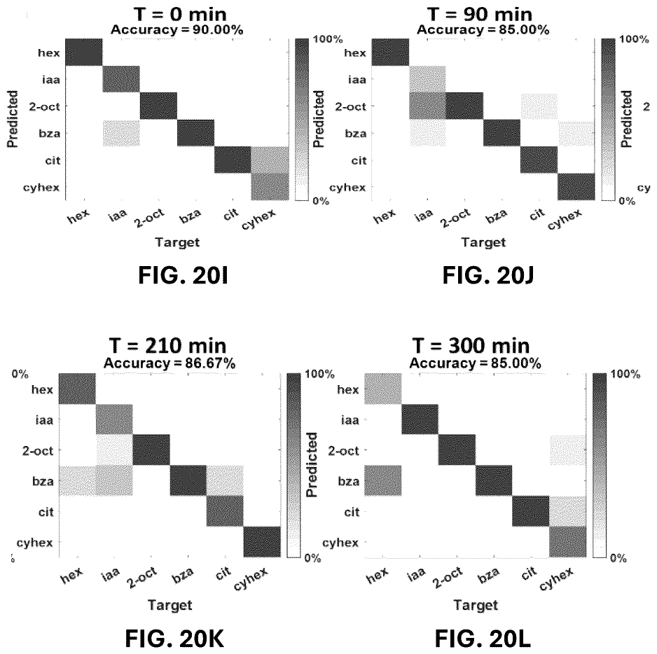

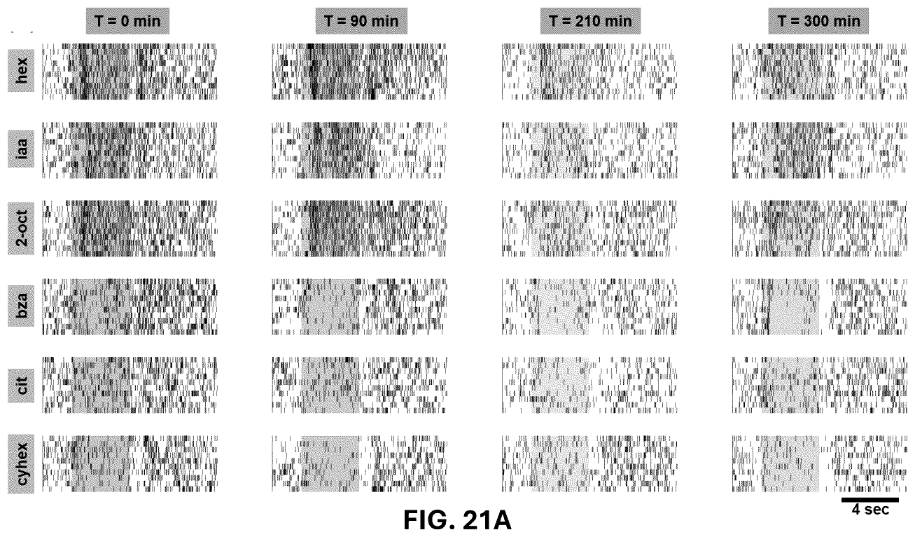

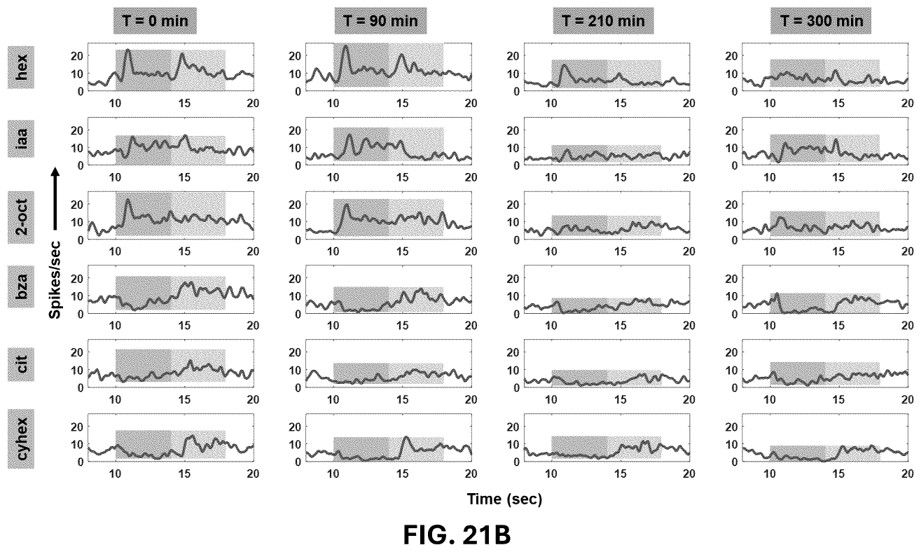

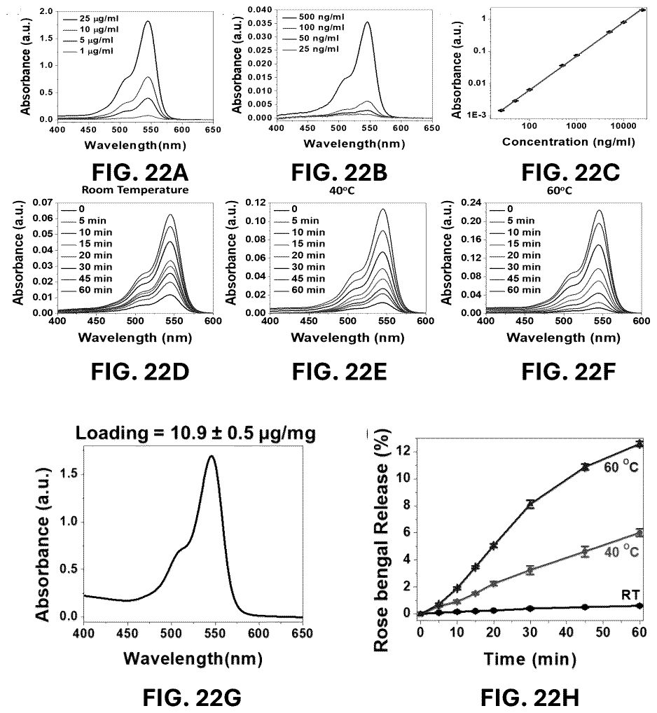

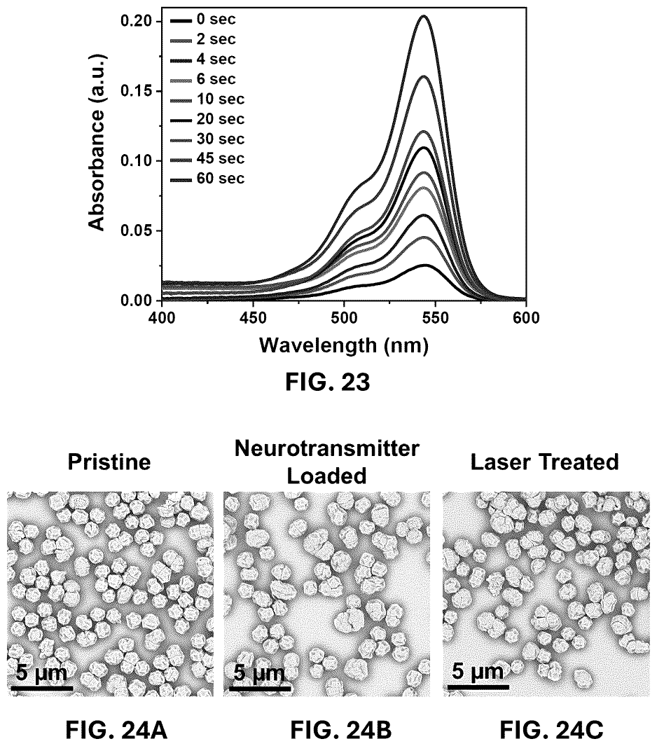

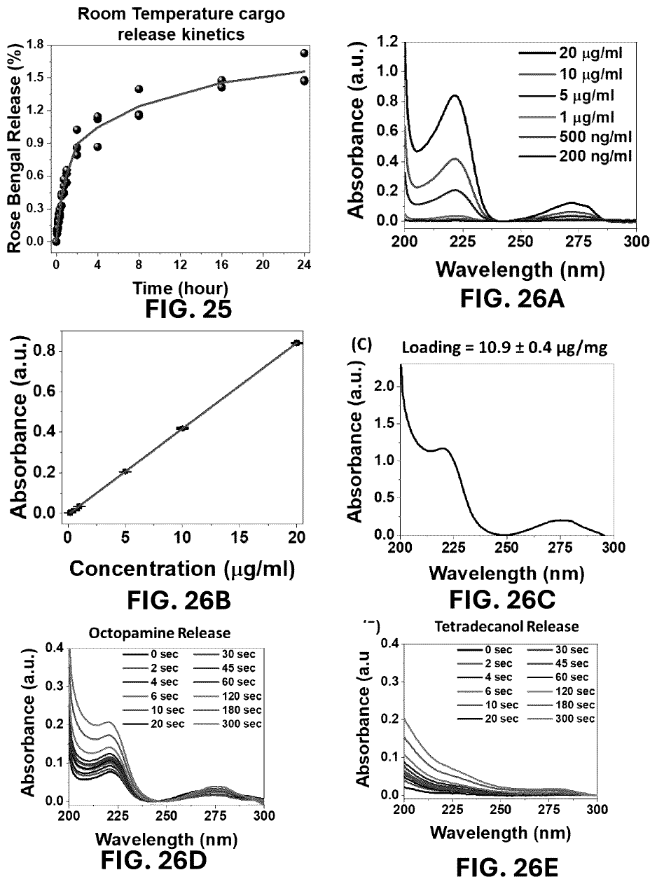

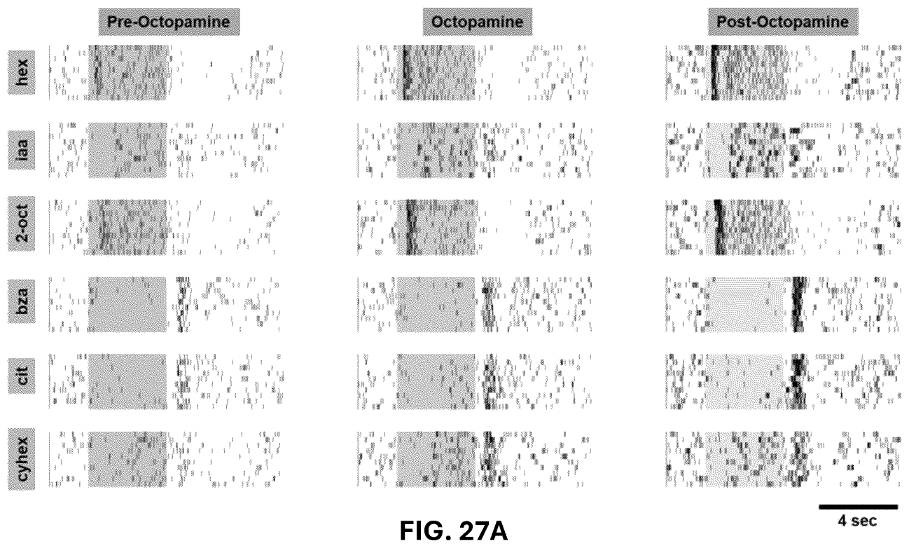

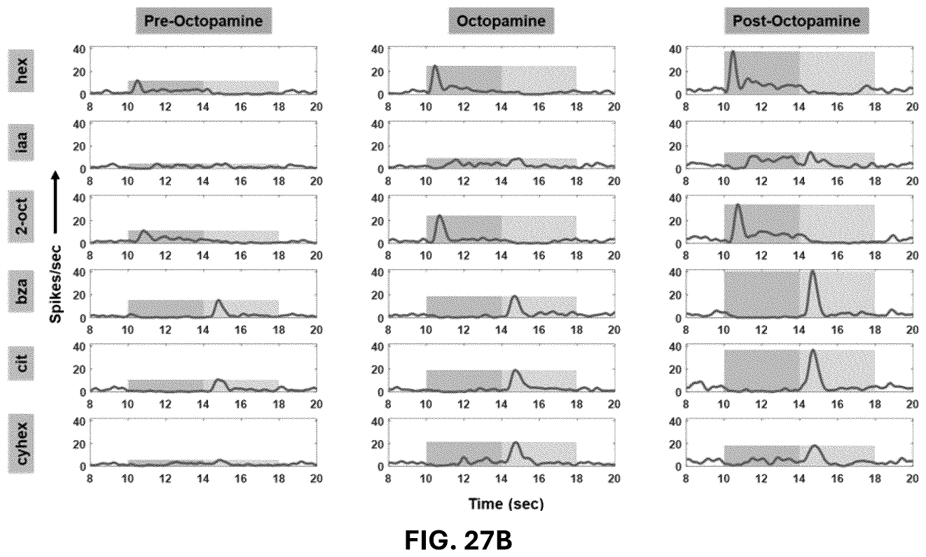

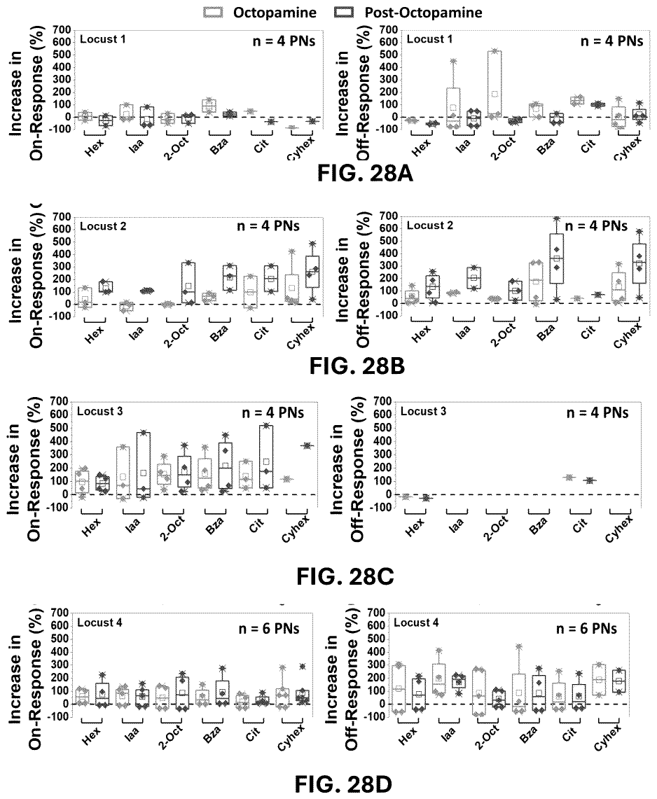

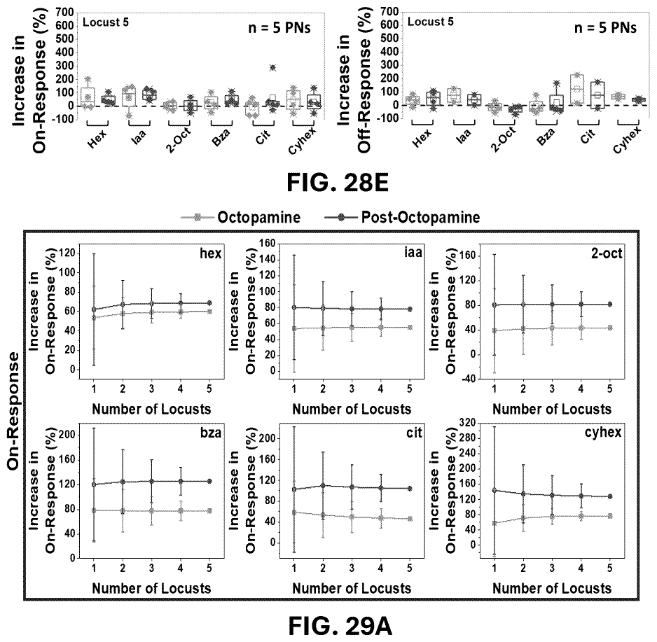

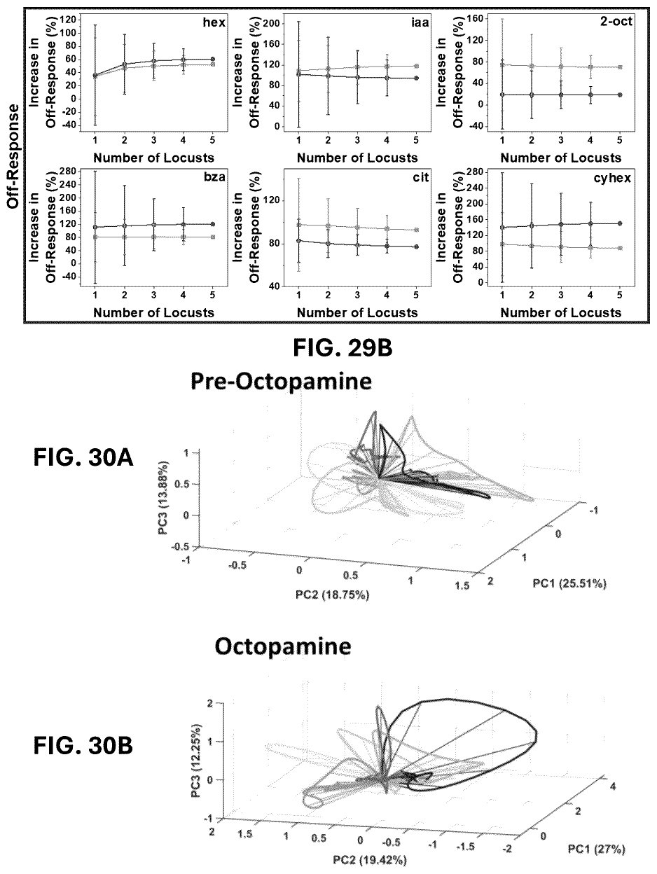

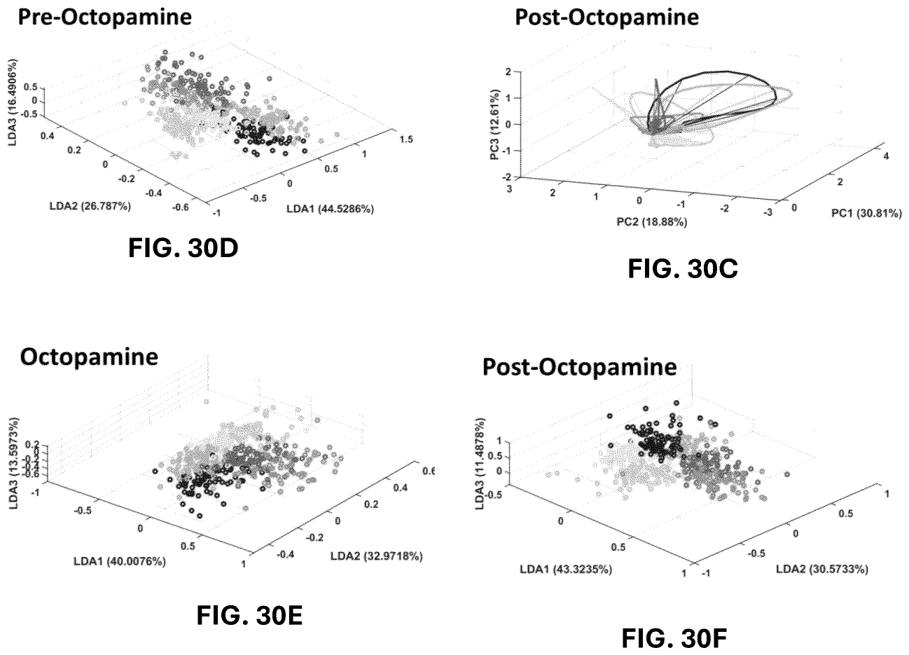

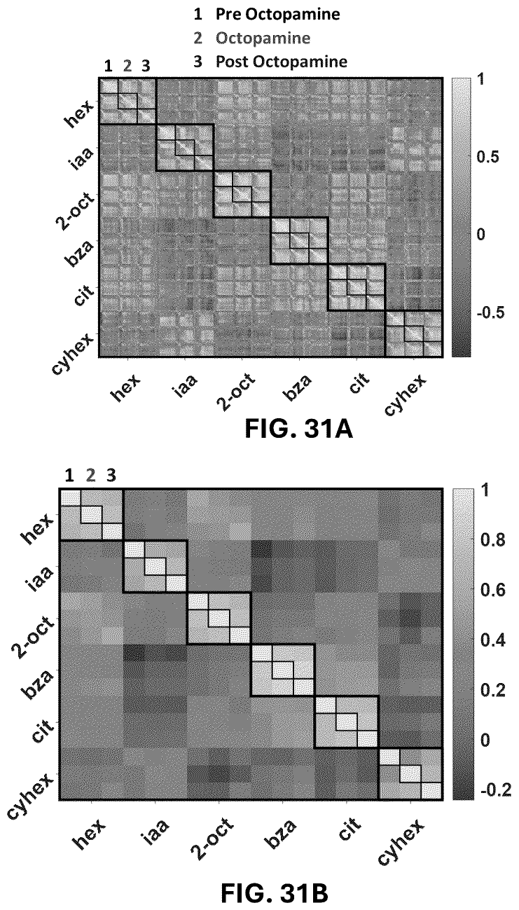

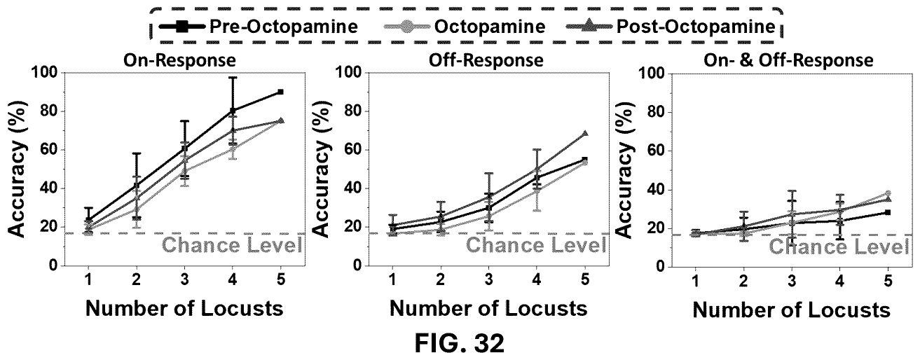

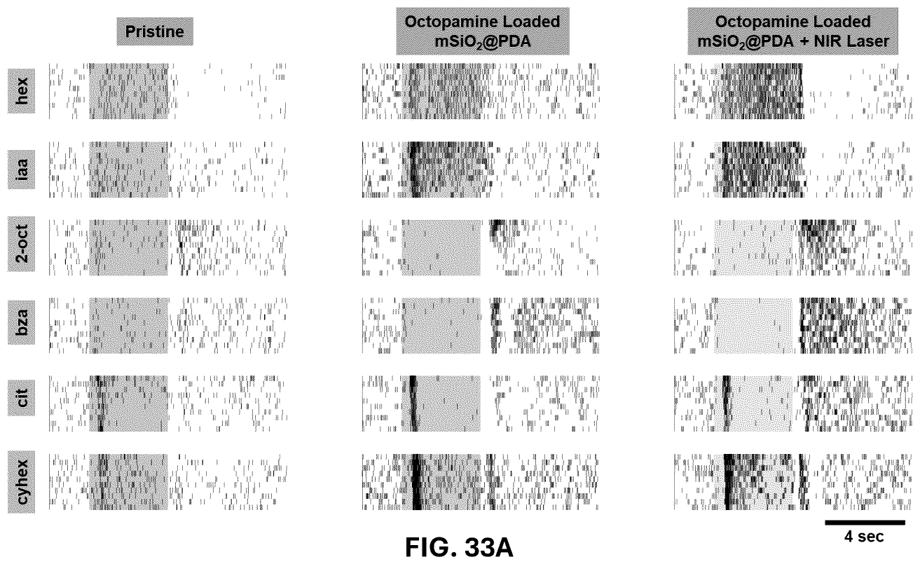

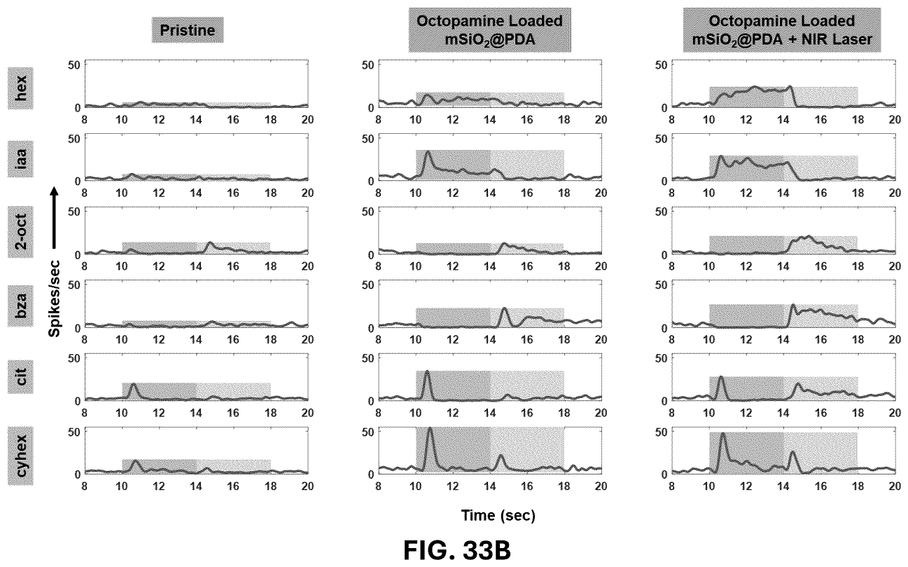

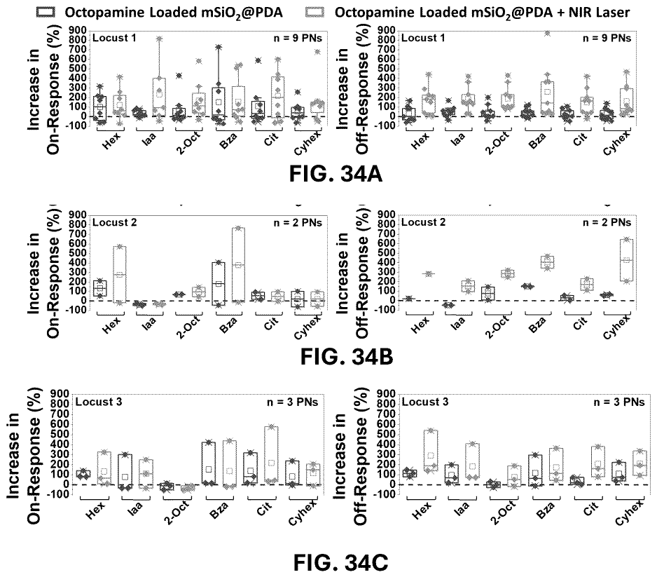

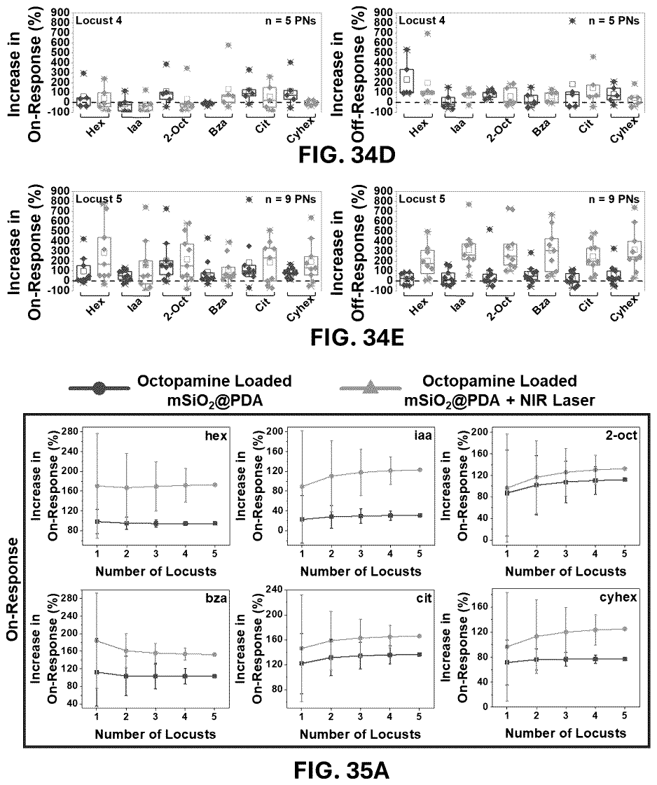

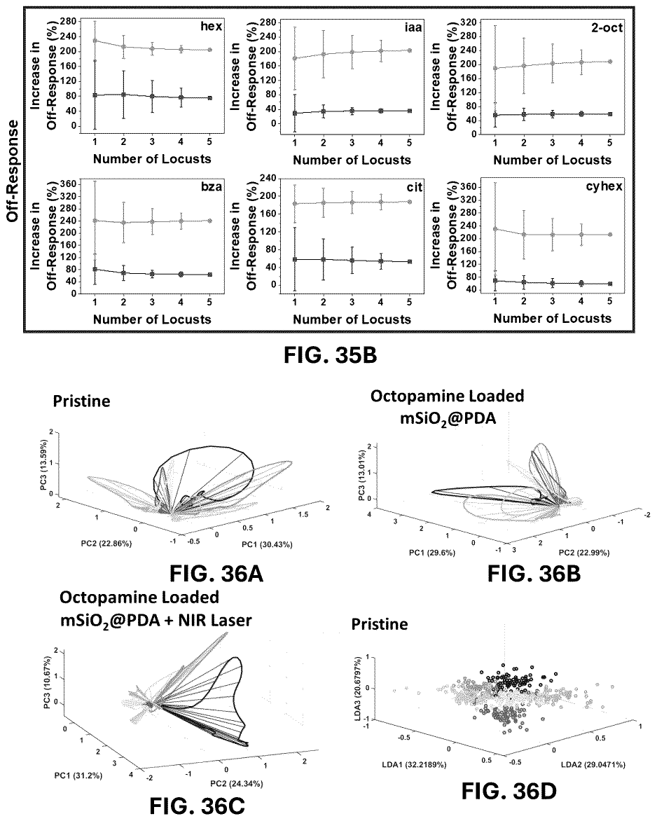

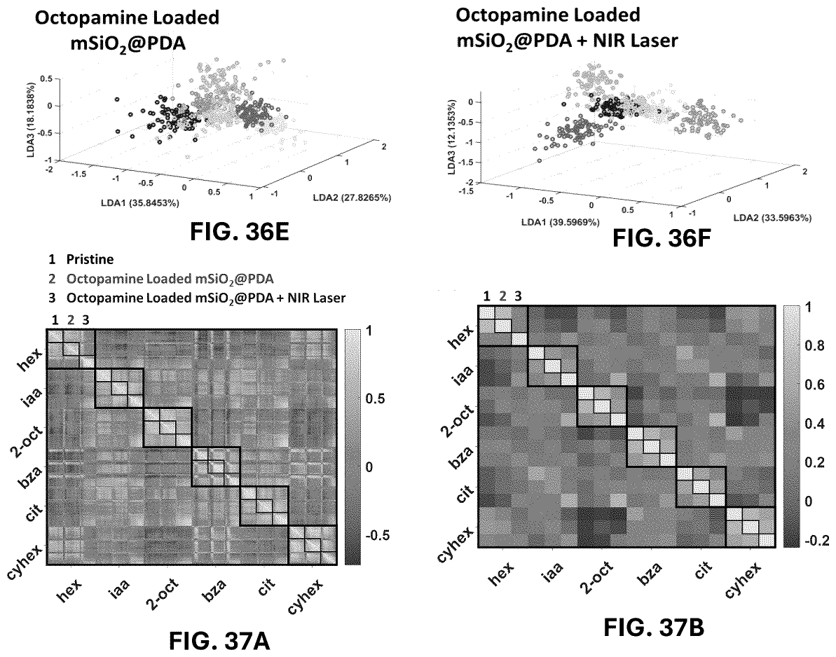

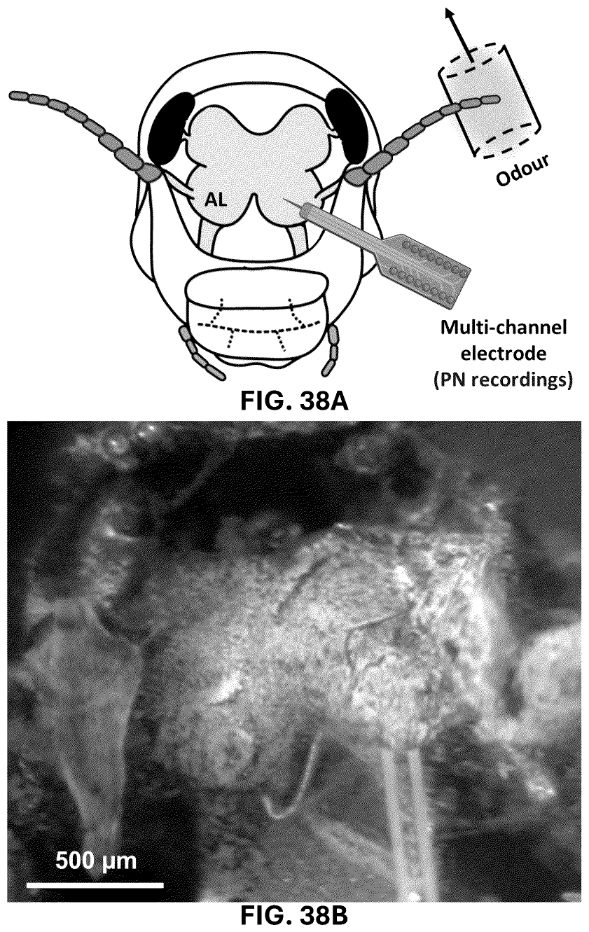

FIG. 21A is a set of raster plots representing spiking response of a projection neuron (PN) is shown. Each row corresponds to one trial and responses in ten trials are shown for each odor. The color bar indicates the time when odorants were puffed onto the locust antenna. Responses to various odor stimulus (hexanol (hex), isoamyl acetate (iaa), 2-octanol (2-oct), benzaldehyde (bza), citral (cit) and cyclohexanone (cyhex)) at T=0 min (first column), T=90 min 25 (second column), T=210 min (third column) and T=300 min (fourth column) are shown as individual blocks from left to right.

FIG. 21B is a set of graphs of corresponding mean spike rate of projection neuron representing on and off response towards various odor stimulus, derived from the raster plots in FIG. 21A. The blue color bar indicates the time when odor stimulus was presented to the locust antenna. The gray color bar indicates the off-response behavior of projection neurons towards odor stimulus.

FIG. 22A is a graph of absorbance spectra of varying concentrations of rose Bengal in water.

FIG. 22B is another graph of absorbance spectra of varying concentrations of rose Bengal in water.

FIG. 22C is a corresponding calibration curve obtained from peak absorbance at 546 nm (Data are presented as mean values+/−SD, n=3 independent experiments).

FIG. 22D is a graph of absorbance spectra of the supernatant decanted from rose Bengal loaded mSiO2@PDA nanoparticles dispersed in water at different time points after incubation at room temperature.

FIG. 22E is a graph of absorbance spectra of the supernatant decanted from rose Bengal loaded mSiO2@PDA nanoparticles dispersed in water at different time points after incubation at 40° C.

FIG. 22F is a graph of absorbance spectra of the supernatant decanted from rose Bengal loaded mSiO2@PDA nanoparticles dispersed in water at different time points after incubation at 60° C.

FIG. 22G is an absorbance spectra of supernatant decanted from rose Bengal loaded mSiO2@PDA nanoparticles after complete release from the particles.

FIG. 22H is a graph of dye release kinetics from mSiO2@PDA nanoparticles loaded with rose bengal (model dye) at different temperatures (Data are presented as mean values+/−SD, n=3 independent experiments).

FIG. 23 is a graph of absorbance spectra of supernatant decanted from rose Bengal loaded mSiO2@PDA nanoparticles dispersed in water after varying duration of NIR laser illumination (808 nm, Power Density: 14 mW/mm²).

FIG. 24A is an SEM micrograph of mSiO2@PDA nanoparticles.

FIG. 24B is an SEM micrograph of octopamine loaded mSiO2@PDA nanoparticles.

FIG. 24C is an SEM micrograph of laser treated mSiO2@PDA nanoparticles. Note that no significant change in the morphology of mSiO2@PDA nanoparticles were observed after octopamine loading and laser illumination, demonstrating excellent structural stability of the developed nano-vehicle. These are representative images from n=2 independent experiments with similar results.

FIG. 25 is a graph of passive release kinetics of Rose Bengal released from mSiO2@PDA nanoparticles at room temperature. (individual data points are depicted as black circles and the release kinetics is traced from the mean depicted as red line, n=3 independent tests).

FIG. 26A is a graph of absorbance spectra of varying concentrations of octopamine in water.

FIG. 26B is a corresponding calibration curve obtained from peak absorbance at 222 nm (Data are presented as mean values+/−SD, n=3 independent experiments) as derived from the spectra in FIG. 26A.

FIG. 26C is an absorbance spectra of supernatant decanted from octopamine loaded mSiO2@PDA nanoparticles after complete release from the particles.

FIG. 26D is a graph of absorbance spectra of supernatant decanted from (D) octopamine loaded mSiO2@PDA nanoparticles dispersed in water after varying duration of NIR laser illumination (808 nm, Power Density: 14 mW/mm²).

FIG. 26E is a graph of absorbance spectra of supernatant decanted from 1-tetradecanol loaded mSiO2@PDA nanoparticles dispersed in water after varying duration of NIR laser illumination (808 nm, Power Density: 14 mW/mm²).

FIG. 27A is a set of raster plots representing spiking response of a projection neuron (PN). Each row corresponds to one trial and responses in ten trials are shown for each odor. The color bar indicates the time when odorants were puffed onto the locust antenna. Responses to various odor stimulus (hexanol (hex), isoamyl acetate (iaa), 2-octanol (2-oct), benzaldehyde (bza), citral (cit) and cyclohexanone (cyhex)) pre-octopamine (first column), with 1 mM octopamine (second column) and after washing (third column) are shown as individual blocks from left to right.

FIG. 27B is a graph of corresponding mean spike rate of projection neuron representing on and off response towards various odor stimulus as derived from the raster plots in FIG. 27A. The blue color bar indicates the time when odor stimulus was presented to the locust antenna. The gray color bar indicates the off-response behavior of projection neurons towards odor stimulus.

FIG. 28A is a whisker plot demonstrating increase in on-response (mean spike rate increase in the 4s odor stimulus period) and off-response (mean spike rate increase in the 4s post odor stimulus period) of locust 1 under various odor stimulus (hexanol (hex), isoamyl acetate (iaa), 2-octanol (2-oct), benzaldehyde (bza), citral (cit) and cyclohexanone (cyhex)) in the presence of 1 mM octopamine and after washing with respect to pre-octopamine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 28B is a whisker plot demonstrating increase in on-response (mean spike rate increase in the 4s odor stimulus period) and off-response (mean spike rate increase in the 4s post odor stimulus period) of locust 2 under various odor stimulus (hexanol (hex), isoamyl acetate (iaa), 2-octanol (2-oct), benzaldehyde (bza), citral (cit) and cyclohexanone (cyhex)) in the presence of 1 mM octopamine and after washing with respect to pre-octopamine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 28C is a whisker plot demonstrating increase in on-response (mean spike rate increase in the 4s odor stimulus period) and off-response (mean spike rate increase in the 4s post odor stimulus period) of locust 3 under various odor stimulus (hexanol (hex), isoamyl acetate (iaa), 2-octanol (2-oct), benzaldehyde (bza), citral (cit) and cyclohexanone (cyhex)) in the presence of 1 mM octopamine and after washing with respect to pre-octopamine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 28D is a whisker plot demonstrating increase in on-response (mean spike rate increase in the 4s odor stimulus period) and off-response (mean spike rate increase in the 4s post odor stimulus period) of locust 4 under various odor stimulus (hexanol (hex), isoamyl acetate (iaa), 2-octanol (2-oct), benzaldehyde (bza), citral (cit) and cyclohexanone (cyhex)) in the presence of 1 mM octopamine and after washing with respect to pre-octopamine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 28E is a whisker plot demonstrating increase in on-response (mean spike rate increase in the 4s odor stimulus period) and off-response (mean spike rate increase in the 4s post odor stimulus period) of locust 5 under various odor stimulus (hexanol (hex), isoamyl acetate (iaa), 2-octanol (2-oct), benzaldehyde (bza), citral (cit) and cyclohexanone (cyhex)) in the presence of 1 mM octopamine and after washing with respect to pre-octopamine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 29A is a set of graphs of locust population analyses on the increase in on-response demonstrating robustness of chemical neuromodulation for augmentation of locust olfaction across individual as well as random subsets of locusts. All possible combinations from 5 locusts were considered for the analyses. The increase in on- and off-response is presented as the mean calculated from the means of each iteration (Data are presented as mean values+/−SD, n=5 iterations (number of locusts=1 or 4) and n=10 iterations (number of locusts=2 or 3), SD was calculated from the accuracy of each iteration).

FIG. 29B is a set of graphs of locust population analyses on the increase in off-response demonstrating robustness of chemical neuromodulation for augmentation of locust olfaction across individual as well as random subsets of locusts. All possible combinations from 5 locusts were considered for the analyses. The increase in on- and off-response is presented as the mean calculated from the means of each iteration (Data are presented as mean values+/−SD, n=5 iterations (number of locusts=1 or 4) and n=10 iterations (number of locusts=2 or 3), SD was calculated from the accuracy of each iteration).

FIG. 30A is a 3D plot of high dimensional responses for various odors (hexanol (hex), isoamyl acetate (iaa), 2-octanol (2-oct), benzaldehyde (bza), citral (cit) and cyclohexanone (cyhex)) pre-octopamine and how these responses evolve over time that are visualized in three dimensions following principal component analysis (see Methods). Numbers within parentheses indicate the variance captured along that axis.

FIG. 30B is a 3D plot of high dimensional responses for various odors (hexanol (hex), isoamyl acetate (iaa), 2-octanol (2-oct), benzaldehyde (bza), citral (cit) and cyclohexanone (cyhex)) with 1 mM octopamine and how these responses evolve over time that are visualized in three dimensions following principal component analysis (see Methods). Numbers within parentheses indicate the variance captured along that axis.

FIG. 30C is a 3D plot of high dimensional responses for various odors (hexanol (hex), isoamyl acetate (iaa), 2-octanol (2-oct), benzaldehyde (bza), citral (cit) and cyclohexanone (cyhex)) after washing and how these responses evolve over time are visualized in three dimensions following principal component analysis (see Methods). Numbers within parentheses indicate the variance captured along that axis.

FIG. 30D is a 3D plot of responses of 23 PNs pre-octopamine during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 30E is a 3D plot of responses of 23 PNs with 1 mM octopamine during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 30F is a 3D plot of responses of 23 PNs after washing during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 31A is a plot of correlations between ensemble response vectors evoked by an odorant in different time bins following stimulus onset. Spike counts were averaged across trials (n=10 trials) for all 23 PNs and used for this analysis. Note that each pixel represents correlation between one ensemble vector with another. Similarly, one row or column represents the correlation between one ensemble vector with all other vectors in the identified time periods (80 ON response vectors). Each square with thicker black boundary represents individual odor and each smaller square with thinner black boundary represents response of PNs (1) pre-octopamine, (2) with 1 mM octopamine and, (3) after washing. The color scheme used for representing the correlation values is shown on the right.

FIG. 31B is a plot of correlations between ensemble odor evoked response of PNs as described in FIG. 31A.

FIG. 32 is a set of graphs of locust population analyses on odor recognition performance demonstrating robustness of chemical neuromodulation for augmentation of locust olfaction across individual as well as random subsets of locusts. All possible combinations from 5 locusts were considered for the analyses. The mean accuracy was calculated from the accuracy of each iteration (Data are presented as mean values+/−SD, n=5 iterations (number of locusts=1 or 4) and n=10 iterations (number of locusts=2 or 3), SD was calculated from the accuracy of each iteration).

FIG. 33A is a set of raster plots representing spiking response of a projection neuron (PN) is shown. Each row corresponds to one trial and responses in ten trials are shown for each odor. The color bar indicates the time when odorants were puffed onto the locust antenna. Responses to various odor stimulus (hexanol (hex), isoamyl acetate (iaa), 2-octanol (2-oct), benzaldehyde (bza), citral (cit) and cyclohexanone (cyhex)) under pristine condition (first column), with 1 mg/ml octopamine loaded mSiO2@PDA nanoparticles (second column) and with NIR laser (power density: 14 mW/mm²) illumination in the presence of octopamine loaded mSiO2@PDA nanoparticles (third column) are shown as individual blocks from left to right.

FIG. 33B is a set of graphs corresponding mean spike rate of projection neuron representing on and off response towards various odor stimulus as derived from the raster plots in FIG. 33A. The blue color bar indicates the time when odor stimulus was presented to the locust antenna. The gray color bar indicates the off-response behavior of projection neurons towards odor stimulus.

FIG. 34A is a whisker plot demonstrating increase in on-response (mean spike rate increase in the 4s odor stimulus period) and off-response (mean spike rate increase in the 4s post odor stimulus period) of locust 1 under various odor stimulus (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and with NIR laser illumination in the presence of octopamine loaded mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 34B is a whisker plot demonstrating increase in on-response (mean spike rate increase in the 4s odor stimulus period) and off-response (mean spike rate increase in the 4s post odor stimulus period) of locust 2 under various odor stimulus (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and with NIR laser illumination in the presence of octopamine loaded mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 34C is a whisker plot demonstrating increase in on-response (mean spike rate increase in the 4s odor stimulus period) and off-response (mean spike rate increase in the 4s post odor stimulus period) of locust 3 under various odor stimulus (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and with NIR laser illumination in the presence of octopamine loaded mSiO2@PDA nanoparticles compared to pristine condition.

The box bounds the interquartile range (IQR) divided by the median, and Tukey-style whiskers extend to a maximum of 1.5×IQR beyond the box. Filled diamonds are sample data points, open square represents mean and cross represents outliers.

FIG. 34D is a whisker plot demonstrating increase in on-response (mean spike rate increase in the 4s odor stimulus period) and off-response (mean spike rate increase in the 4s post odor stimulus period) of locust 4 under various odor stimulus (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and with NIR laser illumination in the presence of octopamine loaded mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 34E is a whisker plot demonstrating increase in on-response (mean spike rate increase in the 4s odor stimulus period) and off-response (mean spike rate increase in the 4s post odor stimulus period) of locust 5 under various odor stimulus (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and with NIR laser illumination in the presence of octopamine loaded mSiO2@PDA nanoparticles compared to pristine condition. The box bounds the interquartile range (IQR) divided by the median, and Tukey-style whiskers extend to a maximum of 1.5×IQR beyond the box. Filled diamonds are sample data points, open square represents mean and cross represents outliers.

FIG. 35A is a set of graphs of locust population analyses on the increase in on-response demonstrating robustness of synergistic photothermal/chemical neuromodulation for augmentation of locust olfaction across individual as well as random subsets of locusts. All possible combinations from 5 locusts were considered for the analyses. The increase in on- and off-response is presented as the mean calculated from the means of each iteration (Data are 46 presented as mean values+/−SD, n=5 iterations (number of locusts=1 or 4) and n=10 iterations (number of locusts=2 or 3), SD was calculated from the accuracy of each iteration).

FIG. 35B is a set of graphs of locust population analyses on the increase in off-response demonstrating robustness of synergistic photothermal/chemical neuromodulation for augmentation of locust olfaction across individual as well as random subsets of locusts. All possible combinations from 5 locusts were considered for the analyses. The increase in on- and off-response is presented as the mean calculated from the means of each iteration (Data are presented as mean values+/−SD, n=5 iterations (number of locusts=1 or 4) and n=10 iterations (number of locusts=2 or 3), SD was calculated from the accuracy of each iteration).

FIG. 36A is a 3D plot of high dimensional responses for various odors (hexanol (hex), isoamyl acetate (iaa), 2-octanol (2-oct), benzaldehyde (bza), citral (cit) and cyclohexanone (cyhex)) under pristine conditions and how these responses evolve over time are visualized in three dimensions following principal component analysis (see Methods in Example 1). Numbers within parentheses indicate the variance captured along that axis.

FIG. 36B is a 3D plot of high dimensional responses for various odors (hexanol (hex), isoamyl acetate (iaa), 2-octanol (2-oct), benzaldehyde (bza), citral (cit) and cyclohexanone (cyhex)) with 1 mg/ml octopamine loaded mSiO2@PDA nanoparticles and how these responses evolve over time are visualized in three dimensions following principal component analysis (see Methods in Example 1). Numbers within parentheses indicate the variance captured along that axis.

FIG. 36C is a 3D plot of high dimensional responses for various odors (hexanol (hex), isoamyl acetate (iaa), 2-octanol (2-oct), benzaldehyde (bza), citral (cit) and cyclohexanone (cyhex)) with NIR laser (power density: 14 mW/mm²) illumination in the presence of octopamine loaded mSiO2@PDA nanoparticles and how these responses evolve over time are visualized in three dimensions following principal component analysis (see Methods in Example 1). Numbers within parentheses indicate the variance captured along that axis.

FIG. 36D is a 3D plot of responses of 28 PNs under pristine conditions during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 36E is a 3D plot of responses of 28 PNs with 1 mg/ml octopamine loaded mSiO2@PDA nanoparticles during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 36F is a 3D plot of responses of 28 PNs with NIR laser (power density: 14 mW/mm²) illumination in the presence of octopamine loaded mSiO2@PDA nanoparticles during odor presentation window (50 ms bin size, over 4 s, total 80 points for each odor) are visualized after linear discriminant analysis (LDA) dimensionality reduction. Numbers within parentheses indicate the variance captured along that axis. The three dimensions onto which the data are projected, maximize the variance between classes and minimize within-class variance. The distinct clustering indicates the feasibility of segregating these odors based on the neural responses they elicited.

FIG. 37A is a plot of correlations between ensemble response vectors evoked by an odorant in different time bins following stimulus onset. Spike counts were averaged across trials (n=10 trials) for all 28 PNs and used for this analysis. Note that each pixel represents correlation between one ensemble vector with another. Similarly, one row or column represents the correlation between one ensemble vector with all other vectors in the identified time periods (80 ON response vectors). Each square with thicker black boundary represents individual odor and each smaller square with thinner black boundary represents response of PNs (1) under pristine condition, (2) with 1 mg/ml octopamine loaded mSiO2@PDA nanoparticles and, (3) with NIR laser (power density: 14 mW/mm²) illumination in the presence of octopamine loaded mSiO2@PDA nanoparticles. The color scheme used for representing the correlation values is shown on the right.

FIG. 37B is a plot of correlations between ensemble odor evoked response of PNs as described in FIG. 37A.

FIG. 38A is a schematic depicting the implantation site of the multichannel electrode for acquiring recordings from projection neurons.

FIG. 38B is an optical image of the locust brain incubated with octopamine loaded mSiO2@PDA nanoparticles and implanted with a 16-channel, 4×4 silicon probe (NeuroNexus) for acquiring recordings from projection neurons.

Those of skill in the art will understand that the drawings, described below, are for illustrative purposes only. The drawings are not intended to limit the scope of the present teachings in any way.

### DETAILED DESCRIPTION OF THE INVENTION

The present disclosure is based, at least in part, on the discovery that using a non-genetic, nano-neuromodulation approach, odor discrimination can be enhanced at any interrogation site where an electrode array is randomly placed in an insect brain. As shown herein, compositions and methods related to augmenting insect olfaction performance through nano-neuromodulation are described.

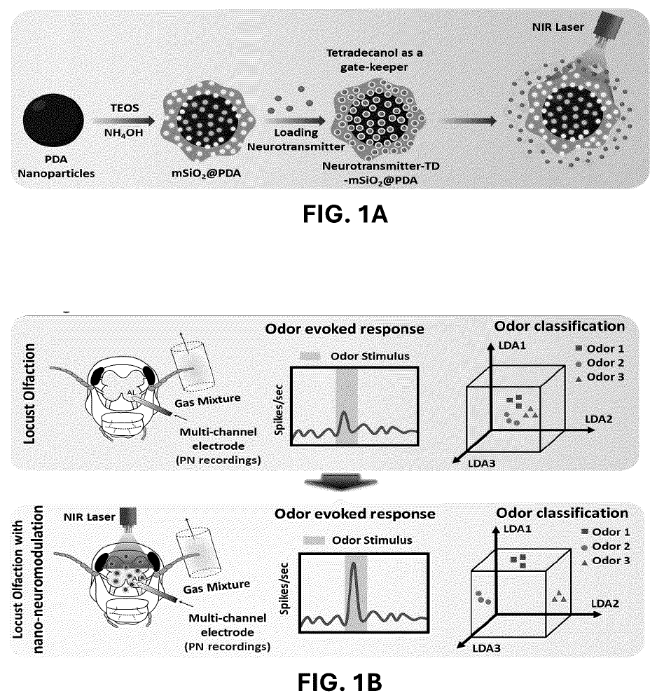

In various aspects, a composition for photothermally triggered delivery of a neurally active compound is disclosed that includes a plurality of functionalized nanoparticles. As illustrated in FIG. 1A, each functionalized nanoparticle includes a nanoparticle core encased in a mesoporous coating formed over the outer surface of the nanoparticle core. The mesoporous coating defines a plurality of nanopores. In various aspects, the plurality of nanopores contains a mixture that includes the neurally active compound and a phase change material. In various aspects, the phase change material is selected to assume a solid phase at an ambient temperature, thereby retaining the mixture within the plurality of nanopores while stored and after administration. The phase change material is further configured to change phase to a liquid phase when the functionalized nanoparticle is heated to a temperature above the melting point of the phase change material, thereby causing the release of the neurally active compound form the functionalized nanoparticle. In various aspects, the functionalized nanoparticle may be heated by illuminating the functionalized nanoparticle using a light source including, but not limited to, a near-infrared laser source.

In various aspects, the nanoparticle core may include nanoparticle cores comprising any suitable biocompatible and biodegradable material without limitation. In some aspects, the nanoparticle core comprises a biocompatible and biodegradable material including, but not limited to, polydopamine (PDA).

In various aspects, the nanoparticle core has a diameter ranging from about 100 nm to about 1000 nm. In various other aspects, the diameter of nanoparticle core may range from about 100 nm to about 200 nm, from about 150 nm to about 250 nm, from about 200 nm to about 300 nm, from about 250 nm to about 350 nm, from about 300 nm to about 400 nm, from about 350 nm to about 450 nm, from about 400 nm to about 500 nm, from about 450 nm to about 550 nm, from about 500 nm to about 600 nm, from about 550 nm to about 650 nm, from about 600 nm to about 700 nm, from about 650 nm to about 750 nm, from about 700 nm to about 800 nm, from about 750 nm to about 850 nm, from about 800 nm to about 900 nm, from about 850 nm to about 950 nm, and from about 900 nm to about 1000 nm. In an exemplary embodiment, the nanoparticle core has a diameter of about 800 nm.

In various aspects, the mesoporous coating may be formed using any suitable biodegradable and biocompatible material without limitation. In one exemplary aspect, the mesoporous coating may be formed using mesoporous silica. In various aspects, the mesoporous coating may have a thickness ranging from about 50 nm to about 1000 nm, from about 75 nm to about 500 nm, from about 100 nm to about 250 nm, from about 100 nm to about 200 nm, and from about 100 nm to about 150 nm. In one exemplary embodiment, the mesoporous coating may have a thickness of about 120 nm.

In various aspects, the plurality of nanopores is monodisperse. In some aspects, the nanopores have a pore diameter ranging from about 1 nm to about 10 nm, from about 2 nm to about 9 nm, from about 3 nm to about 8 nm, from about 4 nm to about 7 nm, and from about 5 nm to about 6 nm. In one exemplary embodiment, the plurality of nanopores may have a pore diameter of about 3 nm.

In various aspects, the mixture contained within the plurality of nanopores of the functionalized nanoparticles includes any suitable phase change material without limitation. In various aspects, the phase change material is selected to be biocompatible and biodegradable with a melting point above the anticipated ambient temperature of the environment within which the disclosed composition is administered. In various aspects, the melting point of the phase change material is above about 25° C., above about 28° C., above about 30° C., above about 35° C., above about 40° C., or above about 45° C.

In various aspects, the phase change material may be a fatty acid or a fatty alcohol that is immiscible with water and has a melting point ranges from about 25° C. to about 60° C. Non-limiting examples of suitable phase change materials include decanoic acid, myristic acid, linolelaidic acid, vaccenic acid, elaidic acid, dodecanol, 1-tridecanol, 1-tetradecanol, hexadecanol, and any combination thereof.

Without being limited to any particular theory, the immiscible property of the selected phase change material provides a means of loading the mixture of the phase change material and neurally active compound into the plurality of nanopores of the functionalized nanoparticles by incubating the functionalized nanoparticles with the mixture in the presence of an organic solvent in which the phase change material is solvent at a temperature above the melting point of the phase change material as described in the examples herein. After a suitable period of incubation, the temperature of the incubation mixture is lowered below the melting point of the phase change material while evaporating the organic solvent, leaving the plurality of functionalized nanoparticles loaded with the mixture as well as excess mixture outside of the nanoparticles. As described in the examples herein, the excess mixture may be removed by rinsing the loaded functionalized nanoparticles with hot water above the melting temperature of the phase change material. Without being limited to any particular theory, the hot water rinses away the mixture, which is in a liquid phase, but the mixture is retained within the nanopores of the functionalized nanoparticles due to the mutual repulsion of the phase change material and the water, which are immiscible.

In various aspects, the mixture contained within the plurality of nanopores of the functionalized nanoparticles further includes any suitable neurally active compound without limitation. In some aspects, the neurally active compound is selected depending on the specific use of the composition, and may be selected from an ion, a neurotransmitter, a candidate compound to treat a neurologic disorder and any combination thereof. In some aspects, the neurally active compound is the candidate compound for treatment of a neurological disorder or a neurally active compound associated with a neurological disorder. Non-limiting examples of suitable neurally active compounds include a potassium ion (K+), octopamine, glycine, glutamate, serotonin, epinephrine, norepinephrine, dopamine, substance P, an opioid compound, ATP, GTP, nitric oxide, gamma amino butyric acid (GABA), and acetylcholine (ACh).

In various aspects, the functionalized nanoparticles of the disclosed composition may be fabricated using any suitable fabrications without limitation. In some aspects, the functionalized nanoparticles of the disclosed composition may be fabricated as described in the examples herein. In this exemplary non-limiting example, polydopamine nanoparticle cores are formed by oxidative self-polymerization of dopamine monomers in a solution comprising water, ethanol, and ammonium, and the polydopamine nanoparticle cores are coated with a porous silica coating formed by incubating the polydopamine nanoparticles in a solution comprising cetyltrimethylammonium bromide, tetraethyl orthosilicate (TEOS), and aqueous ammonia solution. The functionalized nanoparticles are loaded with a mixture of the phase change material and the neurally active compound by incubating the functionalized nanoparticles in the mixture at a temperature above a melting point of the phase change material. The resulting composition may be stored in water at a temperature below the melting point of the phase change material.

In various aspects, the resulting functionalized nanoparticles of the disclosed composition are negatively charged. Without being limited to any particular theory, this negative charge of the functionalized nanoparticles of the disclosed composition may facilitate the selective ionic binding of the functionalized nanoparticles to neurons, which typically have a positive charge. In some aspects, the functionalized nanoparticles of the disclosed may have a zeta potential between about −40 mV and about −30 mV.

In various aspects, the functionalized nanoparticles of the disclosed composition are configured to release at least a portion of the neurally active compound within the plurality of nanopores when the functionalized nanoparticles are heated to a temperature above the melting point of the phase change material, as illustrated in FIG. 1A. The functionalized nanoparticles may be heated by any suitable method without limitation including, but not limited to, illumination by light at a wavelength preferentially absorbed by the functionalized nanoparticles. In some aspects, the light may be near-infrared light, as described in the examples herein (see FIG. 1A). As described in the examples herein, the rate of heating and temperature increase of the composition may depend on any one or more of a plurality of factors including, but not limited to the wavelength of illumination (FIG. 2H), the power of light energy delivered to the composition (FIGS. 11D and 11E), and the concentration of the functionalized nanoparticles in the administered composition (FIGS. 2K and 2L).

In some aspects, the illumination may be applied over several illumination cycles, wherein the composition is illuminated for a period of time sufficient to release a first portion of the neurally active compound, followed by a termination of the illumination causing the cooling of the composition and associated pause in release of the neurally active compound, followed by additional cycles of illumination/release, as illustrated in FIG. 4F.

In various aspects, the disclosed composition may be used to modulate neuroactivity in a preparation that includes a plurality of neurally active cells. In various aspects, the preparation may be any suitable preparation without limitation.

In some aspects, the preparation is an insect olfaction-based chemical sensor that includes a locust with electrodes configured to monitor projection neuron activity within an antennal lobe of the locust, as illustrated in FIG. 1B. As described in the examples herein, the composition is introduced through the cuticle of the locust overlying the antennal lobe, and the functionalized nanoparticles of the composition selectively bind to the neurons within the antennal lobe. As described in the examples, the disclosed composition may include octopamine, a compound that enhances the neural response of the projection neurons of the locust in response to odor stimuli, thereby enhancing the sensitivity of the insect olfaction-based chemical sensor.

In other aspects, the preparation is a brain organoid that includes a plurality of neurons, wherein the brain organoid is mounted within an array of electrodes configured to monitor neural activity in different regions of the brain organoid, as shown illustrated in FIG. 11A. In various aspects, the brain organoid preparation may be used for a variety of purposes depending on the type of neurally active compound administered by the disclosed composition. In some aspects, the neurally active compound may be a candidate compound for treating a neural disorder, and the candidate compound is screened based on changes in neural activity of the brain organoid in response to the candidate compound administered using the disclosed composition. In other aspects, the brain organism may be used as a model for a neural disorder, and the neurally active compound administered using the disclosed composition may be a biomarker or other compound associated with the initiation or progression of the neural disorder; in this aspect, the preparation may be used to characterize biochemical processes associated with a neural disorder.

In the present disclosure, nanomaterial-enabled neuromodulation strategies are exploited to enhance neural signals at a site where electrodes are placed, wherein such increased sensitivity can also lead to enhanced odor discrimination that might be needed for realizing an insect cyborg-based chemical sensing system.

In some aspects, nanomaterial-assisted neuromodulation in augmenting the neural signals in a relatively simple invertebrate model olfactory system (locust, Schistocerca americana) is disclosed. In another aspect, the reversible modulation of neural activity in vitro using polydopamine nanoparticles (PDA NPs) as photothermal nano-transducers is demonstrated. In further aspects, the following two nano-enabled neuromodulation strategies to augment locust olfaction are employed: (1) biocompatible and biodegradable mesoporous silica-coated polydopamine nanoparticles (mSiO2@PDA NPs) as photothermal nano-transducers for photothermal neuromodulation; and (2) locoregional and triggered release of neuromodulators from the porous shells of the core-shell nanostructures at the neural targets in conjunction with photothermal neuromodulation.

In some aspects, polydopamine (core)—mesoporous silica (shell) nanostructures (mSiO2@PDA) can be employed as cargo-carrying nanoparticles that enable on-demand photothermally-triggered neurotransmitter release contained in the mesoporous silica shell owing to the photothermal properties of the PDA nanostructures (FIG. 1A). Spherical PDA nanoparticles with a diameter of 800±32 nm were synthesized via oxidative self-polymerization of dopamine monomer at room temperature in a water-ethanol-ammonium mixture (FIGS. 2A and 2D). Subsequently, a mesoporous silica shell can be formed around PDA nanoparticles via the modified Stöber method. Cetyltrimethylammonium bromide (CTAB), which serves as a porogen, can be added to the reaction mixture to realize nanoscale pores within the silica shell. CTAB can be removed through ion exchange post silica shell formation to realize a mesoporous shell for subsequent neurotransmitter loading.

In an exemplary embodiment, the thickness of the mesoporous silica coating can be ˜ 120 nm (FIG. 2F). A sharp peak at 3.1 nm in the pore size distribution of the mSiO2@PDA nanoparticles confirmed the mesoporous nature of the silica shell (FIG. 2G). Both PDA and mSiO2@PDA nanoparticles exhibited a broad optical absorption band over visible and NIR parts of the electromagnetic spectrum. Both PDA and mSiO2@PDA nanoparticles exhibited negative ζ-potential of −40±3 mV and −30±2 mV (FIG. 21), making them an attractive candidate for nanomaterial-assisted neuromodulation since negatively charged nanoparticles selectively bind to neurons.

In accordance with another aspect, the thermal response of the engineered core-shell nanoparticles to optical stimulus can be characterized. A NIR laser (wavelength of 808 nm) can be employed as an optical stimulus owing to the low optical absorption of biological tissues in the NIR window (650-900 nm). NIR light being a nonionizing radiation poses no risk of tissue damage or genotoxicity. Furthermore, due to its deep tissue penetration capability, NIR light has been readily employed for imaging in clinical applications. To explore the thermal response of mSiO2@PDA nanoparticles under NIR stimulus, different concentrations of mSiO2@PDA nanoparticles dispersed in water were subjected to 808 nm laser irradiation at a power density of 14 mW/mm2.

The temperature rise of core-shell nanoparticles upon NIR stimulus ranges from 25° C. to 55° C. for concentrations ranging from 0.25-2 mg/ml. On the contrary, saline used for electrophysiological recording from locusts exhibited only a 3° C. temperature rise under identical NIR irradiation conditions. The temperature rise at 4 sec can be considered as the primary criterion in determining the optimal nanoparticle concentration. The 4 sec time duration was chosen considering the experimental protocol associated with the odor-evoked spiking response of locusts, 4 sec odor stimuli can be used. The temperature rise for 1 mg/ml of nanoparticles at 4 sec was found to be just above the melting temperature of 1-tetradecanol (FIG. 2L).

In some aspects, the possibility of harnessing mSiO2@PDA nanoparticles to photothermally modulate the odor-evoked spiking response of the locust antennal lobe, which receives information from olfactory receptor neurons in the antenna (FIG. 3A), is described. In another aspect, a standard surgical protocol can be employed, which can be followed by implantation of multi-unit rigid extracellular electrodes in the locust antennal lobe to monitor the response of projection neurons to a wide panel of odors comprised of different functional groups (hexanol (hex)—a primary alcohol, isoamyl acetate (iaa)—an ester, 2-octanol (2-oct)—a secondary alcohol, benzaldehyde (bza)—an aldehyde, citral (cit)—a terpene and cyclohexanone (cyhex)—a ketone) and the odor stimulus can be presented onto the antenna via custom-designed olfactometer. The spiking response of three representative projection neurons in the antennal lobe to hex odor stimulus prior to the administration of the mSiO2@PDA particles is presented in the first column of FIG. 3B. To investigate the effect of laser in the absence of nanoparticles, the laser stimulus was presented simultaneously with the odor and the spiking response was recorded as depicted in the second column. The saline drip was then turned off followed by mSiO2@PDA nanoparticles incubation for 1 hour. The drip was switched back to saline to wash away the unbound mSiO2@PDA nanoparticles and the spiking response was recorded after 30 mins as depicted in column 3 in order to observe the effect of mSiO2@PDA nanoparticles on the locust olfaction. Subsequently, the spiking activity was recorded under 808 nm laser illumination (illuminated only during odor stimulus) as depicted in column 4 in order to investigate the photothermal modulation of the odor-evoked response behavior. The on-response can be defined as the increase in mean spike rate during 4s odor stimulus presentation and the off-response can be defined as the increase in mean spike rate for 4s duration immediately after odor stimulus termination It was observed that the odor-evoked on- and off-response behavior did not change much in the presence of mSiO2-@PDA nanoparticles, however, under laser illumination in the presence of mSiO2@PDA nanoparticles both on- and off-response were significantly enhanced (FIG. 3C, 3D, 14). Moreover, the augmentation of both on- and off-response via photothermal neuromodulation was robust across all individual locusts as well as random subsets of locust populations (FIG. 15-16). It is well known that locusts are ectothermic (cold-blooded) and the excitability of neurons from various regions of the locust nervous system (such as flight neurons and auditory receptor neurons) increases as a function of temperature. Because the neural recording from the locust was performed at room temperature and the maximum temperature during 4s NIR laser illumination is ˜37° C. (FIG. 2L), it is speculated that the increase in both on- and off-response might be attributed to the higher local temperature (photothermal effect) as compared to the ambient temperature, leading to higher excitability and consequent increase in odor-evoked response. Nevertheless, these results indicate that the overall temporal response patterns were preserved but the response strength increased as a result of nano-neuromodulation.

In the present disclosure, odor-evoked responses of recorded neurons were analyzed in the locust antenna lobe to assess the effect of photothermal neuromodulation on odor discrimination (FIG. 17-19). The odor-evoked responses from all 29 neurons can be combined to generate an ensemble vector (see methods) and the activity can be binned in 50 ms time bins. To visualize the responses towards various odors and compare them, these high dimensional responses can be dimensionality reduced using principal component analysis (PCA) and projected into the top three principal component space. It was observed that the response trajectories were non-overlapping for all the odors, suggesting that the odor-evoked responses can be employed for odor discrimination (FIG. 17). This qualitative discriminability was further confirmed via linear discriminant analysis (LDA), which also showed distinct response clusters for the odors in LDA space (FIG. 3E, 3F, 3G, 3H). To quantify the discrimination capability quantitative classification analysis with 5-fold cross-validation (LDA dimensionality reduction followed by quadratic discriminant function classifier) can be employed. It was observed that the odor prediction accuracy based on ensemble spiking response patterns across projection neurons during the on-response period became significantly enhanced due to the photothermal effect (FIG. 3I, 3J, 3K, 3L). Based on the observation of significant enhancement of off-response via photothermal neuromodulation and because both on- and off-response uniquely encode for the identity of the odor stimulus, the effect of photothermal neuromodulation on the odor recognition performance when either on-response or off-response or both are employed for quantitative classification analysis is described. It was observed that photothermal neuromodulation can significantly augment the odor recognition performance when either on-response, off-response, or both was employed for classification analysis at the individual locust level demonstrating the robustness of the neuromodulation effect across individuals (FIG. 19). Moreover, the locust population analyses of odor recognition performance demonstrate that higher classification accuracy can be achieved with a lower number of locusts by employing photothermal neuromodulation. Further, it was observed that both the odor-evoked response and odor recognition performance of the locust reduced slightly over time (FIG. 20-21), thereby further confirming the observed augmentation of locust olfaction resulting from the neuromodulation.

In another aspect, one approach to achieve further response amplification is through the local release of neuromodulators. Therefore, the capability of mSiO2@PDA nanoparticles as a potential cargo carrier was assessed. Rose bengal was employed as a model dye and 1-tetradecanol as a gatekeeper to retain the dye in the nanopores of the silica shell. 1-tetradecanol is a biocompatible phase-change material, which exhibits a melting temperature of 38-39° C. Consequently, 1-tetradecanol can contain the cargo in the nanopores with minimal leakage and enable controlled cargo release upon external stimulation. The dye-loaded mSiO2@PDA nanoparticles exhibited controlled release of dye at 40 and 60° C. with no significant leakage of dye at ambient conditions (25° C.) (FIG. S11 in Appendix A), demonstrating the potential of nano-vehicles for on-demand neuromodulator release. Next, the on-demand release of cargo from the core-shell nanoparticles in response to optical stimulus was investigated. The dye-loaded mSiO2@PDA nanoparticles solution at a concentration of 1 mg/ml was subjected to an NIR stimulus (14 mW/mm²) to trigger the on-demand release. As discussed above, mSiO2@PDA nanoparticles under NIR irradiation at this concentration exhibited a sufficient rise in temperature to cause a phase change of 1-tetradecanol and release the cargo. Under NIR stimulus, the concentration of rose bengal in the surrounding aqueous medium started to increase within 2 sec of irradiation, confirming the rapid on-demand cargo release from the nanoparticles (FIG. 4A, 4B). The cumulative concentration of the released dye steadily increased for the subsequent 1 min indicating the continuous release of the dye from the nanoparticles (FIG. 4B, 23). Finally, to investigate the in vivo on-demand cargo delivery capability, mSiO2@AuNR nanoparticles loaded with IR-650 dye (model cargo) were injected inside the locust cuticle and subjected to NIR stimulus (FIG. 4C). Control groups include locusts without particle injection and locusts with particle injection but no laser irradiation. After applying an 808 nm laser 10 times for 4 secs, the locusts were allowed to freely behave overnight, and then the brain was extracted to measure the dye content. The sheath supporting the brain was removed carefully to ensure only the dye molecules diffused into the brain were accounted for. It was observed that the locust brains without any particle injection showed minimal release after laser irradiation. On the other hand, in locusts injected with the dye-loaded nanoparticles, there was a significant increase in the fluorescence intensity of dye in the brain after laser irradiation (FIG. 4D, 4E). The significant increase in the fluorescence intensity inside the brain demonstrates the successful chemical release of the nanoparticles under optical stimulus. The programmable on-demand stepwise cargo release capability of the nano-vehicle was then investigated. It was observed that the cargo can be released repeatedly in a stepwise fashion from the nano-vehicle under NIR laser pulses (FIG. 4F). The leakage of the cargo in the absence of NIR laser pulses was found to be minimal, establishing the on-demand cargo release capability of the nano-vehicle. The stability of mSiO2@PDA nanoparticles under proposed experimental conditions was further investigated. No significant degradation in the structural integrity of mSiO2@PDA nanoparticles after cargo loading and laser irradiation was observed (FIG. 24). Moreover, only a small fraction (˜1.6%) of cargo (Rose Bengal, model dye) was passively released from the nanoparticles at room temperature demonstrating excellent stability of nanoparticles to hold the cargo within its nanopores (FIG. 25). Octopamine (neurotransmitter) was then employed to investigate the stability of the cargo during the loading and release process. 1H NMR spectra obtained from released octopamine from the octopamine-loaded mSiO2@PDA nanoparticles exhibited characteristic peaks associated with pristine octopamine, thereby demonstrating the chemical stability of the released cargo (FIG. 4G). Octopamine-loaded mSiO2@PDA nanoparticles were further employed to investigate the NIR laser-induced octopamine release kinetics. Similar release kinetics of octopamine were observed as compared to Rose Bengal (FIG. 4H, 26), demonstrating the universality of the proposed payload carrying nano-vehicles. These observations validate the dual-modal response of the core-shell nanoparticles under the optical stimulus, making it a promising candidate for nano-enabled dual-modal neuromodulation that includes both photothermal modulation and local chemical release-based modulation.

Octopamine is a biogenic monoamine, which is well known to act as a neuromodulator, neurotransmitter, and neurohormone in various invertebrate species. Moreover, octopamine has been demonstrated to promote associative learning in the insect brain. Based on these findings, it was surmised that local and controlled release of octopamine can be utilized to modulate the odor-evoked responses in the regions where electrode arrays were randomly placed. The effect of octopamine on the odor-evoked responses from PNs in the locust antennal lobe was investigated (FIG. 5A). The spiking response of three representative PNs in the antennal lobe for hexanol (hex) odor stimulus pre-octopamine treatment is presented in the first column of FIG. 5B. The saline drip was then switched with 1 mM octopamine solution in saline and the spiking response was recorded after 30 mins as depicted in column 2 of FIG. 5B to observe the effect of octopamine on the odor-evoked responses. The drip was switched back to saline to wash away the octopamine and the spiking activity was recorded after 30 minutes as depicted in column 3 to investigate whether the change due to octopamine is reversible and the response goes back to the pre-octopamine condition. The effect of octopamine on the odor-evoked spiking response of projection neurons in the antennal lobe for various odor stimuli (hexanol (hex), benzaldehyde (bza), cyclohexanone (cyhex), isoamyl acetate (iaa), citral (cit) and 2-octanol) was observed (FIG. 27). Both on- and off-response were significantly modulated (either enhanced or suppressed) in the presence of octopamine was observed (FIG. 5C, 5D). Moreover, the observed effect of chemical neuromodulation was similar across individual locusts as well as subsets of locust populations (FIG. 28-29). Similar variability was observed in chemical neuromodulation of neural network activity in honeybee antennal lobe which is speculated to be associated with the plasticity of glomeruli probed during neural activity recording. However, there was no significant difference between octopamine and post-octopamine conditions, indicating a memory effect of octopamine treatment as also observed in the case of honeybees. The effect of chemical neuromodulation on the odor discrimination capability of locust olfaction was further investigated. Linear discrimination analysis was employed on the odor-evoked on-response profile of projection neurons and tested the accuracy in odor prediction using a confusion matrix (FIG. 30-32). It was observed that the odor prediction accuracy based on spiking response from projection neurons decreases due to octopamine treatment, however, after washing the odor prediction accuracy partially returns to the pre-octopamine condition (FIG. 5E, 5F, 5G). This was further confirmed by the population-wise analyses of locust odor recognition performance (FIG. 32). This suggests that octopamine alters the odor-evoked response behavior of locusts. However, octopamine also drastically impairs the odor discrimination capability.

### Brain Organoids

Brain organoids, three-dimensional cultures of neural tissue derived from stem cells, represent a revolutionary platform for studying human brain development, testing drugs, and potentially developing biological computing systems. These “mini-brains” can self-organize into complex neural networks that mimic aspects of human brain function. However, the field faces a significant challenge: how to reliably train and enhance the learning capabilities of these organoid systems without damaging them.

The current state-of-the-art relies primarily on electrical stimulation methods, which present several limitations. Direct electrical stimulation can damage cells, limits the ability to target specific neural populations, and often proves unsuitable for long-term interaction with the tissue. These limitations have become a critical bottleneck in advancing organoid intelligence and its applications in pharmaceutical testing, disease modeling, and bio-computing.

This invention introduces a transformative approach using specially engineered nanoparticles that can selectively target neurons within brain organoids. The innovation lies in the dual-modal nature of these nanoparticles, combining two distinct mechanisms for neural modulation: a physical stimulation method activated by light and a chemical modulation capability. This combination allows for precise, non-invasive control over neural activity while maintaining tissue health over extended periods.

In some aspects, the technology offers several key advantages over existing methods. In other aspects, these advantages can include, but is not limited to, (1) selective targeting of neurons, avoiding interference with other cell types, (2) non-invasive stimulation that preserves tissue integrity, (3) precise spatial and temporal control over neural activation, (4) ability to enhance learning and memory formation through multiple mechanisms, (5) compatibility with long-term training protocols, and (6) scalability for different organoid sizes and configurations.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of enhancing neuronal signaling in a subject in need by administering a therapeutically effective amount of a neuromodulation agent and NIR radiation, so as to modulate neuronal signals.

Methods described herein are generally performed on a subject in need thereof. A subject in need of the therapeutic methods described herein can be a subject having, diagnosed with, suspected of having, or at risk for developing a neuronal disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a neuromodulation agent is, for example, an amount that would cause the desired therapeutic effect in a subject while minimizing undesired side effects. In various embodiments, an effective amount of a neuromodulation agent described herein can substantially enhance neuronal signaling.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a neuromodulation agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to modulate neuronal signals.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Administration of a neuromodulation agent can occur as a single event or over a time course of treatment. For example, a neuromodulation agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to mSiO2@PDA nanoparticles, solubilizers, and electrodes. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001)

Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Augmenting Insect Olfaction Performance Through Nano-Neuromodulation

Biological olfactory systems are highly sensitive and selective and often outperform engineered chemical sensing devices in highly complex and dynamic environments. As a result, chemical sensing approaches that tap into the capabilities of biological systems, especially the chemical sensors and their sophisticated processing machinery, are rapidly emerging as a promising strategy. However, approaches to read out information from biological systems, especially neural signals, tend to be sub-optimal due to the number of electrodes that can be used and where they can be placed. In Example 1, to overcome this sub-optimality in neural information read-out, the feasibility of employing a nano-enabled neuromodulation strategy to augment insect olfaction-based chemical sensors is demonstrated. By harnessing the photothermal properties of nanostructures and releasing a select neuromodulator on-demand, it is shown that the odor-evoked response from the interrogated regions of the insect olfactory system can not only be enhanced but can also improve odor identification. The nano-enabled neuromodulation strategy presented in Example 1 opens novel avenues in realizing tailored cyborg chemical sensing approaches.

### INTRODUCTION

Ultrasensitive and rapid chemical detection and quantification are critical for a wide variety of applications, including biodiagnostics, homeland security and environmental monitoring and industrial process control. However, even after decades of extensive efforts, the performance of artificial chemical sensing systems (‘e-noses’) still pales compared to superior capabilities of their biological counterparts. On several important metrics such as sensitivity, stability, specificity and tolerance to varying background conditions biological olfactory systems exhibit better performance. Given the limitation of e-noses, could hybrid approaches that take advantage of biological capability be designed? Recent results indicate that such a bio-hybrid or cyborg approach do indeed hold promise for application in security and biomedicine.

Although feasible, developing an approach that taps into the biological capabilities poses several challenges. There are two ways to interrogate a biological system, one involving behavioral observations (such as a dog barking or proboscis extension of insects), or by directly measuring signals from the neural circuits encoding olfactory information. Both these approaches present different sets of challenges that must be overcome to realize a viable bio-hybrid chemical sensing solution. First, behavioral read-outs could be confounding as the generated motor response may also be influenced by other sensory and non-sensory information. On the other hand, neural read-outs provide incomplete information as they are restricted by the number of electrodes that can be placed and the region they can be placed in without compromising the biological system. Furthermore, for the neural read-out approach, it might not be known whether the location at which neural tissue is probed can provide the necessary information for dealing with the chemical sensing problem. For example, in the case of invertebrate olfactory systems, olfactory receptor neurons drive stimulus information-rich spatiotemporal neural activity patterns in the downstream neural circuits (antennal lobe). However, depending upon the location where the neural circuit is probed, the neural readout system might not or only poorly extract these information-rich neural patterns for the stimulus of interest.

Could the chemosensory information at the location where the electrodes are placed be enhanced? Such an approach would change a passive approach where information is just read-out into an active one where the capabilities of the neural circuits as a substrate for information processing is fully exploited.

Emerging neuromodulation techniques enable fine control over neural network dynamics by regulating the biophysical and synaptic properties of neurons. Among diverse neuromodulation strategies, nanomaterial assisted non-genetic neuromodulation techniques have gained extensive attention in the recent years owing to their superior spatiotemporal resolution, minimal invasiveness and deep brain stimulation capability. Nanomaterials assisted neuromodulation wirelessly harnesses energy from deep penetrating external source (viz. optical, acoustic and magnetic) and transduces it to physiologically relevant signals recognizable by neurons, thereby allowing for remote biological modulation. Photothermal nanotransducers (such as plasmonic nanostructures, graphene, polydopamine nanoparticles) that convert optical input into neural perturbations have attracted significant attention recently and in both vertebrate and invertebrate models.

On the other hand, neurotransmitters play a crucial role in communication between neurons via signal transduction in living organisms. However, considering the rapid deactivation of neurotransmitters by the enzymes in the biological environments, encapsulating the neurotransmitters within a nano-vehicle is a potential solution to ensure long-term stability. Owing to the importance of neurotransmitters in modulating the neural activity, the development of nanomaterials that enable on-demand selective and stepwise neurotransmitter release with minimal leakage (i.e., unintended release) is of paramount importance. Among diverse nano-vehicles (lipid-based, polymeric and inorganic nanoparticles) that enable cargo delivery, mesoporous silica nanoparticles have been widely employed owing to their high pore volume and cargo loading capacity, biocompatibility and biodegradability. Imparting photothermal properties to mesoporous silica nanoparticles can enable on-demand neurotransmitter release, which in conjunction with controlled non-invasive focal heating can be employed to achieve precise photothermal/chemical modulation of targeted neural network. Consequently, in Example 1, whether these nanomaterial-enabled neuromodulation strategies can be exploited to enhance the neural signals at the site where the electrodes are placed and whether such increased sensitivity can also lead to enhanced odor discrimination that might be needed for realizing an insect cyborg-based chemical sensing system was investigated.

In this study, the feasibility of nanomaterial-assisted neuromodulation in augmenting the neural signals in a relatively simple invertebrate model olfactory system (locust, Schistocerca americana) is demonstrated. The following two nano-enabled neuromodulation strategies to augment locust olfaction were employed: (1) biocompatible and biodegradable mesoporous silica coated polydopamine nanoparticles (mSiO2@PDA NPs) as photothermal nano-transducers for photothermal neuromodulation; and (2) locoregional and triggered release of neuromodulators from the porous shells of the core-shell nanostructures at the neural targets in conjunction with photothermal neuromodulation (FIG. 1). Using this non-genetic, nano-neuromodulation approach, it is demonstrated that odor discrimination can be enhanced at any interrogation site where an electrode array is randomly placed in the insect brain.

### Results

### Synthesis and Structure of Core-Shell Nanoparticles

Polydopamine (core)—mesoporous silica (shell) nanostructures (mSiO2@PDA) were employed as cargo-carrying nanoparticles that enabled on-demand photothermally-triggered neurotransmitter release contained in the mesoporous silica shell owing to the photothermal properties of the PDA nanostructures (FIG. 1A). Spherical PDA nanoparticles with a diameter of 800±32 nm were synthesized via oxidative self-polymerization of dopamine monomer at room temperature in water-ethanol-ammonium mixture (FIGS. 2A and 2D). Subsequently, mesoporous silica shell was formed around PDA nanoparticles via modified Stöber method. Cetyltrimethylammonium bromide (CTAB), which serves as a porogen, was added to the reaction mixture to realize nanoscale pores within the silica shell. CTAB was removed through ion exchange post silica shell formation to realize mesoporous shell for subsequent neurotransmitter loading. The resultant core-shell nanostructures exhibited crumpled surface (FIGS. 2B, 2C, and 2E). The PDA NPs swell in the presence of CTAB and shrink to their original size after CTAB removal (FIG. 12). During the mesoporous silica shell formation, the swelling and shrinking of PDA with CTAB resulted in a mechanical instability and crumpling of the shell. Considering that the buckling was not present in as-synthesized core-shell nanoparticles prior to the porogen removal, these results suggest that the differential stress generated at the PDA and silica interface during porogen removal as a result of shrinkage of the core particle resulted in the buckled structure of the silica shell. This is analogous to the well-known phenomenon of periodic buckling observed in stiff skin layer on compliant substrate subjected to mechanical stress and gyrification. Dynamic light scattering revealed the thickness of the mesoporous silica coating to be ˜120 nm (FIG. 2F). A sharp peak at 3.1 nm in the pore size distribution of the mSiO2@PDA nanoparticles confirmed the mesoporous nature of the silica shell (FIG. 2G). Both PDA and mSiO2@PDA nanoparticles exhibited a broad optical absorption band over visible and NIR parts of the electromagnetic spectrum (FIG. 2H). The shift in the absorption peak of mSiO2@PDA nanoparticles might be attributed to the combined effect of alkalinity and temperature-mediated degradation of PDA nanoparticles during the silica shell formation and porogen removal, respectively. Both PDA and mSiO2@PDA nanoparticles exhibited negative ζ-potential of −40±3 mV and −30±2 mV (FIG. 21), making them an attractive candidate for nanomaterial assisted neuromodulation owing to the fact that negatively charged nanoparticles selectively bind to neurons.

### Photothermal Response of Core-Shell Nanoparticles Towards Optical Stimulus

Next, the thermal response of the engineered core-shell nanoparticles to optical stimulus was investigated. We employed NIR laser (wavelength of 808 nm) was employed as the as optical stimulus owing to the low optical absorption of biological tissues in the NIR window (650-900 nm). NIR light being a nonionizing radiation poses no risk of tissue damage or genotoxicity. Furthermore, due to its deep tissue penetration capability, NIR light have been readily employed for imaging in clinical applications. To explore the thermal response mSiO2@PDA nanoparticles under NIR stimulus, different concentrations of mSiO2@PDA nanoparticles dispersed in water were subjected to 808 nm laser irradiation at a power density of 14 mW/mm². As expected, the maximum temperature rise increased with an increase in nanoparticle concentration (FIGS. 2J and 2K). The temperature rise of core-shell nanoparticles upon NIR stimulus ranged from 25° C. to 55° C. for concentrations ranging from 0.25-2 mg/ml. On the contrary, saline used for electrophysiological recording from locusts exhibited only 3° C. temperature rise under identical NIR irradiation conditions. The temperature rise at 4 sec was considered as the primary criterion in determining the optimal nanoparticle concentration. The 4 sec time duration was chosen considering the experimental protocol associated with odor-evoked spiking response of locusts, a 4 sec odor stimuli was employed (see below). The temperature rise for 1 mg/ml of nanoparticles at 4 sec was found to be just above the melting temperature of 1-tetradecanol (FIG. 2L).

### Photothermal Neuromodulation of Locust Olfaction

Next, the possibility of harnessing mSiO2@PDA nanoparticles to photothermally modulate the odor-evoked spiking response of locust antennal lobe, which receives information from olfactory receptor neurons in the antenna, was investigated (FIG. 3A). A standard surgical protocol was employed, followed by implantation of multi-unit rigid extracellular electrodes in the locust antennal lobe to monitor the response of projection neurons to a wide panel of odors comprising of different functional groups (hexanol (hex)—a primary alcohol, isoamyl acetate (iaa)—an ester, 2-octanol (2-oct)—a secondary alcohol, benzaldehyde (bza)—an aldehyde, citral (cit)—a terpene and cyclohexanone (cyhex)—a ketone) and the odor stimulus was presented onto the antenna via custom-designed olfactometer. The spiking response of three representative projection neurons in the antennal lobe to hex odor stimulus prior to mSiO2@PDA particles administration is presented in the first column of FIG. 3B. To investigate the effect of laser in the absence of nanoparticles, the laser stimulus was presented simultaneously with the odor and the spiking response was recorded as depicted in the second column. The saline drip was then turned off followed by mSiO2@PDA nanoparticles incubation for 1 hour. The drip was switched back to saline to wash away the unbound mSiO2@PDA nanoparticles and the spiking response was recorded after 30 mins as depicted in column 3 in order to observe the effect of mSiO2@PDA nanoparticles on the locust olfaction. Subsequently, the spiking activity was recorded under 808 nm laser illumination (illuminated only during odor stimulus) as depicted in column 4 in order to investigate the photothermal modulation of the odor-evoked response behavior. The on-response was defined as the increase in mean spike rate during 4s odor stimulus presentation and the off-response was defined as the increase in mean spike rate for 4s duration immediately after odor stimulus termination (FIG. 13), see methods section for more details; comparisons with respect to the mean spike rate in 4s duration immediately before the odor stimulus was presented). It was observed that the odor evoked on- and off-response behavior did not change much in the presence of mSiO2@PDA nanoparticles, however, under laser illumination in the presence of mSiO2@PDA nanoparticles both on- and off-response were significantly enhanced (FIG. 3C, 3D, 14). Moreover, the augmentation of both on- and off-response via photothermal neuromodulation was robust across all individual locusts as well as random subsets of locust populations (FIG. 15-16). It is well known that locusts are ectothermic (cold-blooded) and the excitability of neurons from various regions of locust nervous system (such as flight neurons and auditory receptor neurons) increases as a function of temperature. Considering the fact that the neural recording from the locust was performed at room temperature and the maximum temperature during 4s NIR laser illumination is ˜37° C. (FIG. 2L), it is speculated that the increase in both on- and off-response might be attributed to the higher local temperature (photothermal effect) as compared to the ambient temperature, leading to higher excitability and consequent increase in odor-evoked response. However, the mechanistic understanding of the photothermal neuromodulation of ectothermic animals still needs to be further explored. Nevertheless, these results indicate that the overall temporal response patterns were preserved but the response strength increased as a result of nano-neuromodulation.

Does the increase in neural response lead to more enhanced discrimination between odorants? In a recent report, it was demonstrated that the odor evoked spiking response obtained from the locust antennal lobe can be effectively employed to reliably discriminate among various odors. Accordingly, we analyzed odor-evoked responses of recorded neurons in the locust antenna lobe were analyzed to assess the effect of photothermal neuromodulation on the odor discrimination (FIG. 17-19). The odor evoked responses from all neurons were combined to generate an ensemble vector (see methods) and the activity was binned in 50 ms time bins. To visualize the responses towards various odors and compare them, these high dimensional responses were dimensionality reduced using principal component analysis (PCA) and projected into top three principal component space. It was observed that the response trajectories were non-overlapping for all the odors, suggesting that the odor evoked responses can be employed for odor discrimination (FIG. 17). This qualitative discriminability was further confirmed via linear discriminant analysis (LDA), which also showed distinct response clusters for the odors in LDA space (FIG. 3E, 3F, 3G, 3H). To quantify the discrimination capability, quantitative classification analysis with 5-fold cross validation (LDA dimensionality reduction followed by quadratic discriminant function classifier) was employed. It was observed that the odor prediction accuracy based on ensemble spiking response patterns across projection neurons during the on-response period became significantly enhanced due to photothermal effect (FIG. 3I, 3J, 3K, 3L). Based on the observation of significant enhancement of off-response via photothermal neuromodulation and considering the fact that both on- and off-response uniquely encode for the identity of the odor stimulus, the effect of photothermal neuromodulation on the odor recognition performance when either on-response or off-response or both are employed for quantitative classification analysis was investigated. It was observed that photothermal neuromodulation can significantly augment the odor recognition performance when either on-response or off-response or both was employed for classification analysis at individual locust level demonstrating robustness of the neuromodulation effect across individuals (FIG. 19). Moreover, the locust population analyses of odor recognition performance demonstrates that higher classification accuracy can be achieved with lower number of locusts by employing photothermal neuromodulation.

To assess if the order of the neural recordings in the experiment and consequently the differences in the time at which the photothermal neuromodulation was performed resulted in the observed augmentation of locust olfaction, the robustness of locust olfaction as a function of time without any neuromodulation was investigated. It was observed that both the odor-evoked response and odor recognition performance of the locust reduces slightly over time (FIG. 20-21), thereby further confirming the observed augmentation of locust olfaction resulted from the neuromodulation. It is worth nothing that the high odor classification accuracy at T=0 is from the infrequent serendipitous placement of electrodes at optimal locations in the antennal lobe for the odor panel employed in this study, with the caveat that the odor classification accuracy from different sets of neurons should not be compared since the training data from one locust is not transferrable to another. On the other hand, considering the stability of odor-evoked response patterns throughout the experimental period, odor classification accuracy can be compared among the same set of locusts. In sum, these results provide a proof-of-concept demonstration that the nano-enabled photothermally triggered neuromodulation can be successfully employed for augmenting signals recorded by a randomly placed electrode array.

Loading and On-Demand Release of Payload from mSiO2@PDA Nanoparticles

In addition to photo-thermal modulation, could the recorded neural signals be further amplified? One potential approach to achieve further response amplification is through local release of neuromodulators. Therefore, the capability of mSiO2@PDA nanoparticles as a potential cargo carrier was assessed. Rose Bengal was employed as a model dye and 1-tetradecanol as gatekeeper to retain the dye in the nanopores of the silica shell. 1-tetradecanol is a biocompatible phase-change material, which exhibits a melting temperature of 38-39° C. Consequently, 1-tetradecanol can contain the cargo in the nanopores with minimal leakage and enable controlled cargo release upon external stimulation. The dye-loaded mSiO2@PDA nanoparticles exhibited controlled release of dye at 40 and 60° C. with no significant leakage of dye at ambient conditions (25° C.) (FIG. 22), demonstrating the potential of nano-vehicles for on-demand neuromodulator release. Next, the on-demand release of cargo from the core-shell 307 nanoparticles in response to optical stimulus was investigated. The dye-loaded mSiO2@PDA nanoparticles solution at a concentration of 1 mg/ml was subjected to NIR stimulus (14 mW/mm²) to trigger the on-demand release. As discussed above, mSiO2@PDA nanoparticles under NIR irradiation at this concentration exhibited sufficient rise in temperature to cause phase change of 1-tetradecanol and release the cargo. Under NIR stimulus, the concentration of rose bengal in the surrounding aqueous medium started to increase within 2 sec of irradiation, confirming the rapid on-demand cargo release from the nanoparticles (FIG. 4A, 4B). The cumulative concentration of the released dye steadily increased for the subsequent 1 min indicating the continuous release of the dye from the nanoparticles (FIG. 4B, 23). Finally, to investigate the in vivo on-demand cargo delivery capability, mSiO2@AuNR nanoparticles loaded with IR-650 dye (model cargo) were injected inside the locust cuticle and subjected to NIR stimulus (FIG. 4C). Control groups include locusts without particle injection and locusts with particle injection but no laser irradiation. After applying 808 nm laser for 4 sec for 10 times, the locusts were allowed to freely behave overnight, and then the brain was extracted to measure the dye content. The sheath supporting the brain was removed carefully to ensure only the dye molecules diffused into the brain were accounted for. It was observed that the locust brains without any particle injection showed minimal release after laser irradiation. On the other hand, in locusts injected with the dye-loaded nanoparticles there was a significant increase in the fluorescence intensity of dye in the brain after laser irradiation (FIG. 4D, 4E). The significant increase in the fluorescence intensity inside the brain demonstrates the successful chemical release of the nanoparticles under optical stimulus.

The programmable on-demand stepwise cargo release capability of the nano-vehicle was then investigated. It was observed that the cargo can be released repeatedly in stepwise fashion from the nano-vehicle under NIR laser pulses (FIG. 4F). The leakage of the cargo in the absence of NIR laser pulses was found to be minimal, establishing the on-demand cargo release capability of the nano-vehicle. The stability of mSiO2@PDA nanoparticles under proposed experimental conditions was further investigated. It was observed that no significant degradation in the structural integrity of mSiO2@PDA nanoparticles after cargo loading and laser irradiation occurred (FIG. 24). Moreover, only a small fraction (˜1.6%) of cargo (Rose Bengal, model dye) was passively released from the nanoparticles at room temperature demonstrating excellent stability of nanoparticles to hold the cargo within its nanopores (FIG. 25). Octopamine (neurotransmitter) was then employed to investigate the stability of the cargo during loading and release process. 1H NMR spectra obtained from released octopamine from the octopamine loaded mSiO2@PDA nanoparticles (spectra no. 2 depicted in red) exhibited characteristic peaks (peaks for hydrogen atom groups labelled as a, b, c, d and f) associated with pristine octopamine (spectra no. 1 depicted in black), thereby demonstrating the chemical stability of the released cargo (FIG. 4G). Octopamine loaded mSiO2@PDA nanoparticles were further employed to investigate the NIR laser induced octopamine release kinetics. Similar release kinetics of octopamine as compared to Rose Bengal was observed (FIG. 4H, 26), demonstrating the universality of the proposed payload carrying nano-vehicles. These observations validate the dual-modal response of the core-shell nanoparticles under optical stimulus, making it a promising candidate for nano-enabled dual-modal neuromodulation: photothermal modulation and local chemical-release based modulation.

### Chemical (Octopamine) Neuromodulation of Locust Olfaction

Octopamine is a biogenic monoamine, which is well known to act as neuromodulator, neurotransmitter and neurohormone in various invertebrate species. Moreover, octopamine has been demonstrated to promote associative learning in insect brain. Based on these findings, it was surmised that local and controlled release of octopamine can be utilized to modulate the odor-evoked responses in the regions where electrode arrays were randomly placed. The effect of octopamine on the odor-evoked responses from PNs in the locust antennal lobe was first investigated (FIG. 5A). The spiking response of three representative PNs in the antennal lobe for hexanol (hex) odor stimulus pre-octopamine treatment is presented in the first column of FIG. 5B. The saline drip was then switched with 1 mM octopamine solution in saline and the spiking response was recorded after 30 mins as depicted in column 2 in order to observe the effect of octopamine on the odor-evoked responses. The drip was switched back to saline to wash away the octopamine and the spiking activity was recorded after 30 minutes as depicted in column 3 in order to investigate whether the change due to octopamine is reversible and the response goes back to pre-octopamine condition. The effect of octopamine on the odor-evoked spiking response of projection neurons in the antennal lobe for various odor stimulus (hexanol (hex), benzaldehyde (bza), cyclohexanone (cyhex), isoamyl acetate (iaa), citral (cit) and 2-octanol) was observed (FIG. 27). It was observed that both on- and off-response was significantly modulated (either enhanced or suppressed) in the presence of octopamine (FIG. 5C, 5D). Moreover, the observed effect of chemical neuromodulation was similar across individual locusts as well as subsets of locust populations (FIG. 28-29). Similar variability was observed in chemical neuromodulation of neural network activity in honeybee antennal lobe which is speculated to be associated with the plasticity of glomeruli probed during neural activity recording. However, there was no significant difference between octopamine and post-octopamine condition, indicating a memory effect of octopamine treatment as also observed in the case of honeybee. The effect of chemical neuromodulation on the odor discrimination capability of locust olfaction was further investigated. Linear discrimination analysis on the odor-evoked on-response profile of projection neurons was employed and the accuracy in odor prediction using confusion matrix was tested (FIG. 30-32). It was observed that the odor prediction accuracy based on spiking response from projection neurons decreases due to octopamine treatment, however, after wash the odor prediction accuracy partially returns to the pre-octopamine condition (FIG. 5E, 5F, 5G). This was further confirmed by the population wise analyses of locust odor recognition performance (FIG. 32). This suggests that octopamine alters the odor-evoked response behavior of locusts, however it drastically impairs the odor discrimination capability.

### Synergistic Photothermal/Chemical Neuromodulation of Locust Olfaction

Subsequently, it was hypothesized that integrating the photothermal modulation with the chemical modulation could further augment the odor-evoked response of locust while preserving the information content of the neural signals. To test this hypothesis, the m-SiO2@PDA nanoparticles were employed to deliver neurotransmitter (octopamine) on-demand in the locust brain and the synergistic effect of chemical and photothermal neuromodulation on the odor evoked spiking response behavior of the locust was investigated (FIG. 6A). The spiking response of 3 representative projection neurons in the antennal lobe evoked by hexanol (hex) odor stimulus pre-PDA particles treatment is presented in the first column of FIG. 6B. Subsequently, octopamine loaded m-SiO2@PDA nanoparticles were incubated with locust brain for 1 hour. After washing away the free nanoparticles, the spiking response was recorded after 30 mins as depicted in column 2 in order to observe the effect of slow release of octopamine from octopamine loaded m-SiO2@PDA nanoparticles. Subsequently, the spiking activity was recorded under optical stimulus (illuminated only during odor stimulus) as depicted in row 3 in order to investigate the combined effect of photothermal treatment and octopamine release on the odor-evoked response behavior. A significant increase in both the on- and off-response behavior in response to odor stimulus under optical stimulus was observed (FIGS. 6C, 6D, 6E and 33). The effect was robust across all the individual locusts as well as the random population of locusts (FIG. 34-35). LDA was employed to assess the effect of dual-modal neuromodulation on the odor discrimination capability of the locust olfaction (FIG. 36). It was observed that the synergistic effect of neurotransmitter release with the nanoparticles leads to an increase in odor-evoked response without compromising the odor discrimination capability (FIG. 6F, 36-37). Interestingly, the significant increase in off-response observed as a consequence of synergistic photothermal/chemical neuromodulation resulted in augmented odor recognition performance when off-response was also employed for classification analyses (FIG. 6G, 6H). The nano-vehicle assisted dual-modal neuromodulation strategy presented here resulted in augmented odor-evoked response as well as enhanced odor discrimination capability. Furthermore, it is worth noting that the slow release (leakage) of neurotransmitter from nanoparticles even in the absence of an external trigger significantly enhanced both on- and off-response. It indicates that the slow release of neurotransmitter in low concentrations over a long period of time, which mitigates the adverse effects associated with bolus injection of high concentrations of neurotransmitter, is sufficient to enhance the neural response. Taken together, these results demonstrate that the nanoparticles can potentially be employed for both mono- and dual-modal neuromodulation (photo- and chemo-modulation).

### Methods

Synthesis of Polydopamine-Mesoporous Silica Core-Shell Nanoparticles (mSiO2@PDA Nanoparticles)

All chemicals were used as received without further purification unless otherwise mentioned. Polydopamine nanoparticles (employed as core) were synthesized via previously reported oxidative self-polymerization technique. Briefly, 112 ml of ethanol (190 proof, Decon Labs Inc, USA) was mixed with 252 ml of ultrapure water (resistivity of 18.2 MΩ·cm) in a 1 L glass container. Subsequently, 1.12 ml of aqueous ammonia solution (28-30%, NH₄OH, 221228, Sigma, USA) was introduced to the above ethanol/water mixture. After 30 minutes of stirring at room temperature, 28 ml of aqueous dopamine hydrochloride solution (5% W/V, H8502, Sigma, USA) was introduced to the reaction mixture. The reaction was kept under constant gentle stirring for 30 hours at room temperature in an open container. The PDA particles were collected via centrifugation (6000 rpm, 10 minutes) and washed with ultrapure water five times and redispersed in ultrapure water for further use.

Mesoporous silica shell was fabricated around the PDA core via Stöber method with slight modification. Briefly, 4 ml of cetyltrimethylammonium bromide (CTAB, 0.1 M, H5882, Sigma, USA) was added to 8 ml of PDA nanoparticles dispersion (10 mg/ml in ultrapure water) and stirred for 10 minutes at 37° C. Subsequently, 3 ml of tetraethyl orthosilicate (TEOS, 17% V/V in ethanol, 333859, Sigma, USA) was injected in one shot to the above mixture under vigorous stirring. Subsequently, 50 μl of aqueous ammonia solution (28-30%, NH₄OH) was added immediately and the reaction mixture was left under vigorous stirring for 16 hours at room temperature. The core-shell mSiO2@PDA was collected via centrifugation (6000 rpm, 5 minutes) and washed three times with ultrapure water. The collected particles were redispersed in 240 ml of ammonium nitrate solution (NH4NO3, 1% W/V in ethanol, 221244, Sigma, USA) and refluxed at 45° C. for 24 hours to remove CTAB template from the silica shell which was employed as porogen, resulting in mesoporous shell. The obtained mesoporous silica-coated PDA (mSiO2@PDA) nanoparticles were collected via centrifugation (6000 rpm, 5 minutes) and washed sequentially with ethanol and water three times each and redispersed in ethanol for further use.

### Material Characterization

A drop of nanoparticle dispersion in water was drop-casted onto the copper grids (Carbon Type-B, 200 mesh, Ted Pella, USA) and transmission electron microscope (TEM) images were acquired via JEOL JEM-2100F field emission electron microscope. A drop of nanoparticle dispersion was drop-casted onto clean silicon substrate, followed by gold sputtering (5 nm thickness) and scanning electron micrographs (SEM) were acquired via JEOL JSM-7001 LVF Field Emission scanning electron microscope. The zeta potential and dynamic light scattering (DLS) measurements were performed via Malvern Zetasizer (Nano ZS). The absorbance spectra of the nanoparticles were obtained using Shimadzu UV-1800 spectrophotometer. Brunauer-Emmett-Teller (BET) Analyzer (Quantachrome Nova 2000e) was employed to obtain the pore-size distribution of the core-shell nanoparticles.

NIR Induced Photothermal Response of Core-Shell mSiO2@PDA Nanoparticles

Pristine mSiO2@PDA nanoparticles were dispersed in saline solution (utilized for neural activity recordings from locus brain) at varying concentrations (0.25-2 mg/ml) and 150 μl of these dispersed nanoparticles placed in a cap of 1.65 ml polypropylene centrifuge tube. A fiber optic coupled NIR laser diode module (808 nm, continuous wave, 2 W, Power technologies inc.) fitted with a collimator at the end of the optical fiber was employed as optical stimulus source. The NIR laser was placed above the nanoparticle dispersion and the laser power density delivered to the nanoparticles was adjusted to 14 mW/mm² by tuning the laser beam spot size and the distance of the collimator from the nanoparticle dispersion with the help of collimator. The temperature variation of the nanoparticle dispersion under NIR stimulus was monitored over a period of 1 minute via an IR camera (FLIR E6-XT, Teledyne FLIR LLC, USA). The temperature variation of the saline solution was monitored under similar conditions for comparison.

### Cargo Loading and Release Kinetics

The loading of cargo (dye or neurotransmitter) was carried out using 1-tetradecanol as a gatekeeper via previously reported technique with slight modification. Rose Bengal was employed as a model cargo system to investigate the cargo loading capacity and release kinetics of mSiO2@PDA nanoparticles. Briefly, 4 mg of 1-tetradecanol (8.08146, Sigma, USA) was mixed with 50 μl of rose bengal solution (5 mg/ml in ethanol, 330000, Sigma, USA) in a round bottom glass tube under mild stirring at 75° C. for 30 minutes. Subsequently, mSiO2@PDA nanoparticles dispersion (8 mg in 300 μl) was introduced to the mixture and the reaction mixture was kept under mild stirring for additional 2 hours at 90° C. until the ethanol was evaporated completely. The particles were redispersed in hot water (1 ml, 80° C.), sonicated for 5 sec, and immediately centrifuged (6000 rpm, 3 minutes) to collect the cargo loaded mSiO2@PDA nanoparticles. The supernatant was decanted, and the particles were redispersed in cold water. The cargo loaded particles were washed with cold water 10 times to ensure complete removal of any free dye in the solution. The dye loaded particles were redispersed in water and stored at 4° C. for further use. The cargo loading capacity of mSiO2@PDA nanoparticles was assessed by dispersing the dye loaded particles in acetone, followed by sonication for 30 minutes to ensure complete removal of dye molecules from the nanoparticles. The UV-Vis absorbance spectra of the supernatant collected after centrifugation (6000 rpm, 3 minutes) was measured and the absorbance at 546 nm was employed to estimate the dye loading capacity of the mSiO2@PDA nanoparticles. The dye loading capacity is estimated as follows:

$$Loading capacity = \frac{mass of dye loaded ( μg )}{mass of nanoparticles ( mg )}$$

The cargo release kinetics of the mSiO2@PDA nanoparticles under thermal stimulus was assessed via subjecting the dye loaded nanoparticles to room temperature, 40° C. and 60° C. for various time periods. The UV-vis absorbance spectra of the supernatant were measured, and the dye release was estimated as follows:

$$Dye release ( \% ) = \frac{Dye released in the supernatant ( μg )}{total dye loaded in the nanoparticle ( μg )} \times 100$$

Similar protocol was employed for loading IR 650 dye (Fluoroprobes, USA) and octopamine (O0250, Sigma, USA) in mSiO2@PDA nanoparticles.

### Photothermal Cargo Release Kinetics and On-Demand Release

For probing the photothermally triggered dye release from mSiO2@PDA nanoparticles, rose bengal was employed as a model cargo. Briefly, 200 μl of rose bengal loaded mSiO2@PDA nanoparticle dispersion (1 mg/ml in saline solution) was irradiated by NIR laser (808 nm) at a power density of 14 mW/mm² for varying time periods. Subsequently, the supernatant was collected via centrifugation (6000 rpm, 3 minutes). The UV-vis absorbance spectra of the supernatant were measured, and the dye release was estimated following the similar protocol as mentioned in previous section. A similar protocol was employed for investigating the NIR laser induced octopamine release kinetics. For 1H NMR analysis, supernatant was decanted from octopamine loaded mSiO2@PDA nanoparticles after complete release from the particles and lyophilized. Subsequently, samples were dissolved in DMSO-d6 (156914, Sigma, USA) at approximately 5 mg/ml. 1H NMR were run on Agilent DD2 500 MHZ NMR spectrometer. All spectra were processed using Bukner Topsin Version 4.1.1.

To assess the on-demand photothermally triggered release of cargo in-vivo, IR 650 dye was employed as a model cargo. Briefly, 1 mg of IR650 loaded mSiO2@PDA nanoparticles was dispersed in 10 μl of saline solution and injected inside the locust near brain region through cuticle using 31-gauge insulin syringe. Care was taken to prevent damaging the brain and the neural sheath encapsulating the brain. Subsequently, 1-day post-injection, the locust was irradiated with NIR laser (808 nm) at a power density of 14 mW/mm² for 40 seconds (10 pulses of 4 seconds with inter-pulse width of 1 minute) to probe on-demand dye release. After 1 hour of photothermal treatment, the locust brain was extracted, and care was taken to remove the neural sheath completely in order to ensure that only dye released onto the brain was monitored. The extracted brain was rinsed thoroughly with saline and placed in clean glass slides with minimum amount of saline so as to prevent dehydration during measurements. Finally, the fluorescence map of the extracted brains was obtained using LICOR Odyssey CLx scanner.

### Electrophysiological Recordings

Young-adult (post-fifth instar) locusts (Schistocerca americana) of either sex raised in a crowded colony were employed for electrophysiological recordings as described in a previous report. Briefly, the locusts were immobilized with both antennae intact. The antennal lobe region of the brain was exposed, desheathed (after treatment with protease) and superfused with locust saline using a saline drip system (to maintain continuous flow of fresh saline) at room temperature. For acquiring extracellular multiunit recordings from the projection neurons (PNs), a 16-channel, 4×4 silicon probe (NeuroNexus) was placed in the superficial layers of antenna lobe (FIG. 38). Electrode contact pads were electroplated with gold via electrochemical workstation to maintain impedances in 200-300 kΩ range. Extracellular signals from PNs were amplified with a gain of 10k and filtered between 0.3-6 kHz via a custom-made 16-channel amplifier (Biology Electronics Shop; Caltech, Pasadena, CA). The signals were then acquired at a 15 kHz sampling rate using a custom-built MATLAB program. The single unit responses were generated by performing off-line spike sorting as described before. Subsequently, the spikes were counted and binned in 50 ms time-bins.

### Odor Stimulation

Odor stimulations were delivered to the locust antenna using a standard procedure. Briefly, odors were diluted in mineral oil (1% V/V) and placed in a sealed dark bottle with an inlet and outlet ports. A constant volume (0.1 L/min) of static headspace above the diluted odor-mineral mixture was displaced into a filtered and desiccated carrier air stream (0.75 L/min) via a custom-built olfactometer (SMC valves, NI-DAQ controller) which was automated and triggered using MATLAB code to minimize the interference during recordings. The odors were presented in a pseudo-randomized order with odor stimulus duration of 4 sec. Each odor was presented for 10 trials with inter-trial interval of 60 sec. A large vacuum funnel was placed right behind the locust preparation to allow for continuous removal of delivered odor. The odor panel used for all the experiments in this study are as follows: (1) hexanol (H13303, Sigma, USA), (2) isoamyl acetate (W205508, Sigma USA), (3) 2-octanol (O4504, Sigma, USA), (4) benzaldehyde (B1334, Sigma, USA), (5) citral (C83007, Sigma, USA), and (6) cyclohexanone (398241, Sigma, USA).

### Photothermal, Chemical and Dual-Modal Neuromodulation

Prior to starting the electrophysiological recordings, the NIR laser (808 nm) was aligned directly above the locust brain and the delivered laser power density was tuned to 14 mW/mm². A TTL controlled mechanical shutter was placed directly in between the laser and the locust brain to control the laser irradiation period. The shutter was controlled using NI-DAQ controller and triggered using MATLAB code (similar to olfactometer). The laser stimulus was presented at the same time as the odor stimulus. To minimize the interference during electrophysiological recordings, the laser and shutter system was set-up ahead of the recordings. The electrophysiological recordings from the PNs of the locust in response to the odor stimulus was first acquired prior to neuromodulation experiments. After 30 minutes of first set of recordings, the NIR laser (808 nm) at a power density of 14 mW/mm² was turned on and the electrophysiological recordings in response to odor stimulus were performed following similar protocol to assess the effect of NIR laser on the odor evoked responses. The laser was then turned off after the recording is completed. Subsequently, 1 mg/ml mSiO2@PDA nanoparticles were introduced in the locust brain after ceasing the continuous flow of locust saline through the locust preparation and incubated for 1 hour. The locust saline drip was turned on to wash off the excess unbound particles from the locust preparation and the electrophysiological recordings were acquired following similar protocol to assess the effect of nanoparticles on the odor evoked responses. After 30 minutes of this set of recordings, the laser was turned on again in order to present both laser and odor stimulus together, and the electrophysiological recordings were acquired using similar protocol to assess the effect of photothermal neuromodulation on the odor evoked response of locust. In the case of both chemical and dual-modal neuromodulation, similar experimental protocol was employed. However, in case of chemical stimulation, laser stimulus was completely removed.

### Determination of Odor Evoked On- and Off-Response

For analyses, first the time binned spike counts obtained after spike sorting was aligned with respect to the odor presentation and then averaged across trials for each odor and PNs. Subsequently, a matrix was constructed with these trial-averaged spike counts for each odor. This resulted in 6 different matrix corresponding to each odor comprising of a block of n rows that represented the trail-averaged spike counts from n different PNs (n=29 for photothermal neuromodulation, n=28 for dual-modal neuromodulation and n=23 for chemical neuromodulation experiments). Odor evoked on-response was defined as the mean spike rate during the entire duration of odor presentation and the off response was defined as mean spike rate in the 4s duration immediately after the odor stimulus was removed. For a PN to be considered to have on- or off-response, the mean spike rate during on-period or off-period must exceed 3 times the standard deviation of the mean spike rate in the 4s duration immediately before the odor presentation (FIG. 13). The change in on-response or off-response resulting from various neuromodulation strategies was calculated as follows:

$$Change in Response ( \% ) = \frac{A - B}{B} \times 100$$

where, A is the on- or off-response during neuromodulation and B is the on- or off-response in pristine condition when no neuromodulation strategy was employed. For analyzing statistical differences between two groups, paired two-samples t-test was employed.

For locust population analyses to assess the variability and robustness across individual locusts, a random subset of locusts was chosen (from n locusts choose k random locusts; k was varied) and the mean change in response was calculated from all the projection neurons. This was done for all the possible combinations for k and the mean and standard deviations of these combinations were calculated and plotted in FIG. 16, 29, 35.

### Dimensionality Reduction Analyses

To visualize the variation in responses from all the PNs towards various odors, two kinds of dimensionality analyses were employed: principal component analysis (PCA) and linear discriminant analysis (LDA). In both cases, only responses during the odor presentation were considered (4s odor stimulation; 80 time bins). A response matrix was composed for all responses in a following fashion. Responses obtained from different PNs were first aligned with respect to odor on-set and averaged across trials which resulted in a matrix comprising of 80 columns representing trial-averaged responses in different PNs (each row) towards a single odor. Subsequently, the responses towards different odors were concatenated resulting in a high dimensional data matrix with 80×6 (480, 6 odors) columns with each row belonging to individual PNs.

First, PCA was employed to visualize the changes in ensemble neural response with respect to time (i.e., trajectory in state space). For PCA dimensionality reduction, the data covariance matrix was computed and projected onto three principal eigen vectors which captured maximum variance in the response. The odor trajectories were first smoothed using five-point moving average low-pass filter and visualized by connecting temporally consecutive points (FIGS. 17, 30, and 36).

For multi class LDA analysis, each column of the matrix was first labelled depending upon the odor identity and then the variance within class (S^{(w)}) and variance between class (S^{(b)}) were estimated as follows:

$${S}^{( w )} = {\sum }_{i = 1}^{L} {\sum }_{j , {y}_{j} = i} ( {x}_{j} - {μ}_{i} ) {( {x}_{j} - {μ}_{i} )}^{T}$$

$${S}^{( b )} = {\sum }_{i = 1}^{L} {n}_{i} ( {μ}_{i} - μ ) {( {μ}_{i} - μ )}^{T}$$

where, xᵢ is i^{th} column vector in data matrix, yᵢ is the corresponding label, L is the number of classes, μᵢ is the mean of points in class i, μ is mean of all points and nᵢ is the number of points in class i. The eigenvectors and corresponding eigenvalues of S^{(w)-1}*S^{(b)} matrix was computed, and the data points were then projected onto three eigenvectors (color-coded by their stimulus) which corresponds to the largest eigenvalues (FIGS. 3E-H, 17, 20-E-H, 30, and 36). These eigenvectors represent maximum variance among classes and minimum within-class variance.

### Classification Analyses

5-fold cross validation analysis was employed to estimate the classification accuracy by randomly splitting the dataset into 5 separate equal groups. The observations were first labelled as described above in the LDA analyses. In each iteration, one of the 5 groups of two trial data from all the odors were removed to be utilized as test data while the remaining data was used as training set. The training set was fit using a quadratic discriminant where the neural responses in one time bin were predictors and the class label for that time bin was the expected classifier output, which resulted in 80 predicted responses for a test trial of one odor. The mode of all the 80 predicted responses was taken as the predicted class label for that odor. To visualize the prediction accuracy of the classifier, a confusion matrix was generated comparing the known responses with the predicted responses, where a fully diagonal matrix represents 100% classification accuracy. Finally, the classification accuracy was computed by taking the average of the diagonal elements of the confusion matrix.

For locust population analyses to assess the robustness across individual locusts, a random subset of locusts was chosen (from n locusts choose k random locusts; k was varied) and the signals from different locusts were combined. The classification analyses were repeated and the mean and standard deviations of the accuracies for each group size from all possible combinations were calculated and plotted.

### DISCUSSION

Biological sensory systems have far superior capabilities compared to their engineered counterparts on several tasks. Tapping into these sensing capabilities is a viable strategy for applications where other solutions do not exist. This is certainly the case in chemical sensing where a general purpose, non-invasive chemical sensing devices are yet to be realized. While behavioral readouts from animals have been used effectively in some cases (dogs barking or insect proboscis extension), directly reading out neural signals from the appropriate sensory regions provides an alternate approach for creating bio-hybrid sensing systems. A key challenge in developing a neural interface for biosensing is that the electrode arrays used are randomly placed and spatially limited in the signals they can record. In this Example, a dual modulation approach to augment signals picked up near an extracellular electrode array is presented. First, using nanomaterials that directly modulate neural response through photothermal effect, improved odor discrimination was shown (FIG. 3). Second, using an orthogonal chemo-modulation approach, local delivery of a neuromodulator was shown, which in combination with the photothermal modulation provided further enhancement (FIG. 6). Together, the presented approach provides a route for realizing robust interfaces between the engineered and the biological systems.

In a previous study, it was demonstrated that PDA nanoparticles that have a negative surface charge selectively attached themselves to hippocampal neurons in an in vitro assay. Furthermore, it was shown that these nanoparticles could photothermally modulate the bound neurons and suppress their spiking activity. The results in this Example here have demonstrated an opposite effect, where the neural spiking activity became enhanced after photothermal modulation. The precise mechanisms that caused these results are yet to be determined. However, one possibility is that the in vivo insect olfactory neural network has densely connected, non-spiking GABAergic local neurons which may have been suppressed more by nano-neuro modulation. The resulting release from inhibition could have caused the excitatory neurons to fire more action potentials. Irrespective of the underlying mechanism, the classification analyses revealed that the net-effect was robust enhancement in odor discrimination information.

While the enhancement through photo-thermal modulation was sufficient for the odor discrimination task targeted herein, an additional strategy was examined to further enhance the overall sensing performance. Such capability might be needed for a larger odor panel or potentially improve other analytical metrics such as lower limit of detection. In this second approach, in addition to their ability to convert light to heat, the cargo-carrying capability of a nanostructure comprising a photothermal PDA core and mesoporous silica shell was also exploited. The nano-heaters were loaded with a neuromodulator (octopamine) and local and focal release of this chemical modulator was demonstrated. The results showed that the chemo-modulation by itself did not enhance the information content of the neural signals recorded (FIGS. 5, 27, and 32). However, in combination, the photo- and chemo-modulation of neural activity provided significant boost to the overall odor discrimination performance (FIG. 6, 33-37).

It is worth noting that the neuromodulation strategies presented here exhibited both increase and decrease in odor-evoked responses. Moreover, the modulation level varies highly across the different odors tested here. Similar variability was observed in chemical neuromodulation of neural network activity in honeybee antennal lobe, which is suggested to be associated with the plasticity of glomeruli probed during neural activity recording. Consequently, the results indicate that the nano-enabled neuromodulation creates a filter for odor patterns, which potentially resulted in an enhanced odor-discrimination performance.

For the purposes of this work, these nanoparticles were injected directly into the target neural circuit. In an earlier work, it was shown that the aerosolized delivery of nanoparticles to the antenna could provide an alternate approach to deliver the selected nano-vehicle into the insect brain. Such an approach could lend itself to high-throughput nano-augmentation of the several insect brains in parallel. However, it would still be important to place the electrode arrays close enough to a region of the brain with the nano-vehicles carrying payloads to fully exploit the photo- and chemo-modulatory potential.

The high stability of response patterns over a recording session could enable acquiring training data for target chemicals prior to the deployment of the insect cyborgs and used over the entire deployment period (typically a few hours), especially with the augmentation strategy demonstrated here.

It is believed that the nano-enabled neuromodulation strategies for augmentation of biological systems demonstrated herein can overcome several incessant bottlenecks in realizing ultrasensitive autonomous insect cyborg-based chemical sensing system with high specificity. Furthermore, it is believed that the nanostructure design presented herein is ubiquitously applicable and amenable for loading wide repertoire of neuromodulators to realize on-demand synergistic neuromodulation of targeted neural circuits in a tether-free fashion. The unique design and versatility of the reported nanoparticles can be harnessed for neuromodulation therapy acting as a powerful tool for neurotherapeutics as well as understanding neural pathways.

### Further Details

Mesoporous silica shell was formed around PDA nanoparticles via modified Stöber method. Cetyltrimethylammonium bromide (CTAB), which serves as a porogen, was added to the reaction mixture to realize nanoscale pores within the silica shell. CTAB was removed through ion exchange post silica shell formation to realize mesoporous shell for subsequent neurotransmitter loading. The resultant core-shell nanostructures exhibited crumpled surface (FIG. 2B, 2C, 2E). The PDA NPs swell in the presence of CTAB and shrink to their original size after CTAB removal (FIG. 12). During the mesoporous silica shell formation, the swelling and shrinking of PDA with CTAB resulted in a mechanical instability and crumpling of the shell. Considering that the buckling was not present in as-synthesized core-shell nanoparticles prior to the porogen removal, these results suggest that the differential stress generated at the PDA and silica interface during porogen removal as a result of shrinkage of the core particle resulted in the buckled structure of the silica shell. This is analogous to the well-known phenomenon of periodic buckling observed in stiff skin layer on compliant substrate subjected to mechanical stress and gyrification.

A NIR laser (wavelength of 808 nm) was employed as the optical stimulus owing to the low optical absorption of biological tissues in the NIR window (650-900 nm). NIR light is a nonionizing radiation and poses no risk of tissue damage or genotoxicity. Furthermore, due to its deep tissue penetration capability, NIR light have been readily employed for imaging in clinical applications.

The spiking response of three representative projection neurons in the antennal lobe to hex odor stimulus prior to mSiO2@PDA particles administration is presented in the first column of FIG. 3B. To investigate the effect of laser in the absence of nanoparticles, the laser stimulus was presented simultaneously with the odor and the spiking response was recorded as depicted in the second column. The saline drip was then turned off followed by mSiO2@PDA nanoparticles incubation for 1 hour. The drip was switched back to saline to wash away the unbound mSiO2@PDA nanoparticles and the spiking response was recorded after 30 mins as depicted in column 3 in order to observe the effect of mSiO2@PDA nanoparticles on the locust olfaction. Subsequently, the spiking activity was recorded under 808 nm laser illumination (illuminated only during odor stimulus) as depicted in column 4 in order to investigate the photothermal modulation of the odor-evoked response behavior. The on-response was defined as the increase in mean spike rate during 4s odor stimulus presentation and the off-response as the increase in mean spike rate for 4s duration immediately after odor stimulus termination (FIG. 13, see methods section for more details; comparisons with respect to the mean spike rate in 4s duration immediately before the odor stimulus was presented).

It is well known that locusts are ectothermic (cold-blooded) and the excitability of neurons from various regions of locust nervous system (such as flight neurons and auditory receptor neurons) increases as a function of temperature. Considering the fact that the neural recording from the locust was performed at room temperature and the maximum temperature during 4s NIR laser illumination is ˜37° C. (FIG. 2L), it is speculated that the increase in both on- and off-response might be attributed to the higher local temperature (photothermal effect) as compared to the ambient temperature, leading to higher excitability and consequent increase in odor-evoked response. However, the mechanistic understanding of the photothermal neuromodulation of ectothermic animals still needs to be further explored. Nevertheless, these results indicate that the overall temporal response patterns were preserved but the response strength increased as a result of nano-neuromodulation.

To assess if the order of the neural recordings in the experiment and consequently the differences in the time at which the photothermal neuromodulation was performed resulted in the observed augmentation of locust olfaction, the robustness of locust olfaction was investigated as a function of time without any neuromodulation. It was observed that both the odor-evoked response and odor recognition performance of the locust reduces slightly over time (FIG. 20-21), thereby further confirming the observed augmentation of locust olfaction resulted from the neuromodulation. It is worth nothing that the high odor classification accuracy at T=0 is from the infrequent serendipitous placement of electrodes at optimal locations in the antennal lobe for the odor panel employed in this study, with the caveat that the odor classification accuracy from different sets of neurons should not be compared since the training data from one locust is not transferrable to another. On the other hand, considering the stability of odor-evoked response patterns throughout the experimental period, odor classification accuracy can be compared among the same set of locusts. In sum, these results provide a proof-of-concept demonstration that the nano-enabled photothermally triggered neuromodulation can be successfully employed for augmenting signals recorded by a randomly placed electrode array.

To assess the capability of mSiO2@PDA nanoparticles as a potential cargo carrier, rose Bengal was employed as a model dye and 1-tetradecanol as gatekeeper to retain the dye in the nanopores of the silica shell. 1-tetradecanol is a biocompatible phase-change material, which exhibits a melting temperature of 38-39° C. Consequently, 1-tetradecanol can contain the cargo in the nanopores with minimal leakage and enable controlled cargo release upon external stimulation. The dye-loaded mSiO2@PDA nanoparticles exhibited controlled release of dye at 40 and 60° C. with minimal leakage of dye at ambient conditions (25° C.) (FIG. 22), demonstrating the potential of nano-vehicles for on-demand neuromodulator release. Next, the on-demand release of cargo from the core-shell nanoparticles in response to optical stimulus was investigated. The dye-loaded mSiO2@PDA nanoparticles solution at a concentration of 1 mg/ml was subjected to NIR stimulus (14 mW/mm²) to trigger the on-demand release. As discussed above, mSiO2@PDA nanoparticles under NIR irradiation at this concentration exhibited sufficient rise in temperature to cause phase change of 1-tetradecanol and release the cargo. Under NIR stimulus, the concentration of rose bengal in the surrounding aqueous medium started to increase within 2 sec of irradiation, confirming the rapid on-demand cargo release from the nanoparticles (FIG. 4A, 4B). The cumulative concentration of the released dye steadily increased for the subsequent 1 min indicating the continuous release of the dye from the nanoparticles (FIGS. 4B and 23). Then the programmable on-demand stepwise cargo release capability of the nano-vehicle was investigated. It was observed that the cargo can be released repeatedly in stepwise fashion from the nano-vehicle under NIR laser pulses (FIG. 4F). The leakage of the cargo in the absence of NIR laser pulses was found to be minimal, establishing the on-demand cargo release capability of the nano-vehicle.

To investigate the in vivo on-demand cargo delivery capability, mSiO2@PDA nanoparticles loaded with IR-650 dye (model cargo) were injected inside the locust cuticle and subjected to NIR stimulus (FIG. 4C). Control groups include locusts without particle injection and locusts with particle injection but no laser irradiation. After applying 808 nm laser for 4 sec for 10 times, the locusts were allowed to freely behave overnight, and then the brain was extracted to measure the dye content. The sheath supporting the brain was removed carefully to ensure only the dye molecules diffused into the brain were accounted for. It was observed that the locust brains without any particle injection showed minimal release after laser irradiation. On the other hand, in locusts injected with the dye-loaded nanoparticles there was a significant increase in the fluorescence intensity of dye in the brain after laser irradiation (FIG. 4D, 4E). The significant increase in the fluorescence intensity inside the brain demonstrates the successful chemical release of the nanoparticles under optical stimulus.

The stability of mSiO2@PDA nanoparticles under proposed experimental conditions was investigated. No significant degradation in the structural integrity of mSiO2@PDA nanoparticles after cargo loading and laser irradiation (FIG. 24) was observed. Moreover, only a small fraction (˜1.6%) of cargo (Rose Bengal, model dye) was passively released from the nanoparticles at room temperature demonstrating excellent stability of nanoparticles to hold the cargo within its nanopores (FIG. 25). Octopamine (neurotransmitter) was then employed to investigate the stability of the cargo during loading and release process. 1H NMR spectra obtained from released octopamine from the octopamine loaded mSiO2@PDA nanoparticles (spectra no. 2 depicted in red) exhibited characteristic peaks (peaks for hydrogen atom groups labelled as a, b, c, d and f) associated with pristine octopamine (spectra no. 1 depicted in black), thereby demonstrating the chemical stability of the released cargo (FIG. 4G).

The spiking response of three representative PNs in the antennal lobe for hexanol (hex) odor stimulus pre-octopamine treatment is presented in the first column of FIG. 5B. The saline drip was then switched with 1 mM octopamine solution in saline and the spiking response was recorded after 30 mins as depicted in column 2 in order to observe the effect of octopamine on the odor-evoked responses. The drip was switched back to saline to wash away the octopamine and the spiking activity was recorded after 30 minutes as depicted in column 3 in order to investigate whether the change due to octopamine is reversible and the response goes back to pre-octopamine condition. The effect of octopamine on the odor-evoked spiking response of projection neurons in the antennal lobe for various odor stimulus (hexanol (hex), benzaldehyde (bza), cyclohexanone (cyhex), isoamyl acetate (iaa), citral (cit) and 2-octanol) was observed (FIG. 27).

Similar variability was observed in chemical neuromodulation of neural network activity in honeybee antennal lobe which is speculated to be associated with the plasticity of glomeruli probed during neural activity recording. However, there was no significant difference between octopamine and post-octopamine condition, indicating a memory effect of octopamine treatment as also observed in the case of honeybee.

The spiking response of 3 representative projection neurons in the antennal lobe evoked by hexanol (hex) odor stimulus pre-PDA particles treatment is presented in the first column of FIG. 6B. Subsequently, octopamine loaded m-SiO2@PDA nanoparticles was incubated with locust brain for 1 hour. After washing away the free nanoparticles, the spiking response was recorded after 30 mins as depicted in column 2 in order to observe the effect of slow release of octopamine from octopamine loaded m-SiO2@PDA nanoparticles. Subsequently, the spiking activity was recorded under optical stimulus (illuminated only during odor stimulus) as depicted in row 3 in order to investigate the combined effect of photothermal treatment and octopamine release on the odor-evoked response behavior.

For the purposes of this work, the nanoparticles were injected directly into the target neural circuit. In an earlier work, it was shown that the aerosolized delivery of nanoparticles to the antenna could provide an alternate approach to deliver the selected nano-vehicle into the insect brain. Such an approach could lend itself to high-throughput nano-augmentation of the several insect brains in parallel. However, it would still be important to place the electrode arrays close enough to a region of the brain with the nano-vehicles carrying payloads to fully exploit the photo- and chemo-modulatory potential.

What is claimed is:

1. A composition for photothermally triggered delivery of a neurally active compound, the composition comprising a plurality of functionalized nanoparticles, each functionalized nanoparticle comprising:
a. a nanoparticle core comprising an outer surface;
b. a mesoporous coating formed over the outer surface of the nanoparticle core, the mesoporous coating defining a plurality of nanopores; and
c. a mixture contained within the plurality of nanopores of the mesoporous coating, the mixture comprising the neurally active compound and a phase change material, wherein the plurality of nanoparticles is configured to release the neurally active compound from the plurality of pores at a temperature above a melting point of the phase change material.

2. The composition of claim 1, wherein the nanoparticle core comprises a biocompatible and biodegradable polymer comprising polydopamine (PDA).

3. The composition of claim 1, wherein the mesoporous coating comprises a biocompatible and biodegradable coating material comprising mesoporous silica.

4. The composition of claim 1, wherein the phase change material comprises a fatty acid or a fatty alcohol that is immiscible with water and the melting point of the phase change material ranges from about 25° C. to about 60° C.

5. The composition of claim 4, wherein the phase change material is selected from decanoic acid, myristic acid, linolelaidic acid, vaccenic acid, elaidic acid, dodecanol, 1-tridecanol, 1-tetradecanol, hexadecanol, and any combination thereof.

6. The composition of claim 1, wherein the neurally active compound is selected from an ion, a neurotransmitter, a compound to treat a neurologic disorder and any combination thereof.

7. The composition of claim 1, wherein the neurally active compound is selected from a potassium ion (K+), octopamine, glycine, glutamate, serotonin, epinephrine, norepinephrine, dopamine, substance P, an opioid compound, ATP, GTP, nitric oxide, gamma amino butyric acid (GABA), and acetylcholine (ACh).

8. The composition of claim 1, wherein the neurally active compound is octopamine.

9. The composition of claim 1, wherein the organic nanoparticle core comprises a diameter of about 800 nm.

10. The composition of claim 1, wherein the mesoporous coating comprises silica with a thickness of about 120 nm defining a plurality of nanopores comprise an average pore diameter of about 3 nm.

11. The composition of claim 1, wherein the functionalized nanoparticle has a zeta potential between −40 and −30 mV.

12. A method to synthesize a functionalized nanoparticle for thermally activated delivery of a neurally active compound, the method comprising:
a. forming a polydopamine nanoparticle core by oxidative self-polymerization of dopamine monomers in a solution comprising water, ethanol, and ammonium;
b. forming a porous silica coating defining a plurality of nanopores over the polydopamine nanoparticle core by incubating the polydopamine nanoparticle in a solution comprising cetyltrimethylammonium bromide, tetraethyl orthosilicate (TEOS), and aqueous ammonia solution;
c. forming the functionalized nanoparticle by loading a mixture comprising the neurally active compound and a phase change material into the plurality of nanopores by incubating the nanoparticle in the mixture at a temperature above a melting point of the phase change material; and
d. storing the nanoparticle in water at a temperature below the melting point of the phase change material to retain the mixture within the plurality of nanopores.

13. A method of modulating neural activity of a preparation comprising a plurality of neural cells, the method comprising:
a. providing a composition comprising a plurality of functionalized nanoparticles, each functionalized nanoparticle comprising a nanoparticle core encased in a mesoporous coating comprising a plurality of nanopores, and a mixture comprising a neurally active compound and a phase change material contained within the plurality of nanopores of the mesoporous coating, wherein the neurally active compound is configured to modulate the neural activity of the neural cells of the preparation;
b. administering a therapeutic amount of the composition to the preparation; and
c. illuminating the therapeutic amount of the composition with NIR light energy to raise the temperature of the functionalized nanoparticles to a temperature above a melting point of the phase change material, to release at least a portion of the neurally active compound to the plurality of neural cells of the preparation.

14. The method of claim 13, wherein illuminating the therapeutic amount of the composition with NIR light energy further comprises using an NIR laser source with a center wavelength of about 800 nm

15. The method of claim 14, wherein the NIR laser source produces NIR light energy at a power ranging from about between 8.33 mW/mm² to about 33 mW/mm².

16. The method of claim 13, wherein the phase change material comprises a fatty acid or a fatty alcohol that is immiscible with water and the melting point of the phase change material ranges from about 25° C. to about 60° C.

17. The method of claim 13, wherein the neurally active compound is selected from an ion, a neurotransmitter, a compound to treat a neurologic disorder and any combination thereof.

18. The method of claim 13, wherein the preparation is a brain organoid and the neurally active compound comprises a candidate compound for treatment of a neurological disorder or a neurally active compound associated with a neurological disorder.

19. The method of claim 13, wherein the preparation is an insect olfaction-based chemical sensor comprising a locust with electrodes monitoring projection neuron activity within an antennal lobe of the locust, and the neurally active compound is octopamine.

20. The method of claim 13, further comprising:
a. terminating the illumination of the therapeutic amount of the composition with the NIR light energy to lower the temperature of the functionalized nanoparticles to a temperature below the melting point of the phase change material, stopping the release of the neurally active compound from the functionalized nanoparticles; and
b. illuminating the therapeutic amount of the composition with NIR light energy to raise the temperature of the functionalized nanoparticles to a temperature above a melting point of the phase change material, to release a second portion of the neurally active compound to the plurality of neural cells of the preparation.
